(19) United States
(12) Patent Application Publication
(10) Pub. No.: US 2013/0315934 A1
(43) Pub. Date: Nov. 28, 2013

(54) Methods and Compositions for Assessing and Treating Cancer

(71) Applicants: Ruth Halaban, New Haven, CT (US); Michael Krauthammer, New Haven, CT (US); Joseph Schlessinger, Woodbridge, CT (US); Titus J. Boggon, Milford, CT (US)

(72) Inventors: Ruth Halaban, New Haven, CT (US); Michael Krauthammer, New Haven, CT (US); Joseph Schlessinger, Woodbridge, CT (US); Titus J. Boggon, Milford, CT (US)

(73) Assignee: Yale University, New Haven, CT (US)

(21) Appl. No.: 13/881,623

(22) PCT Filed: Oct. 28, 2011
(86) PCT No.: PCT/US11/58278

§ 371 Date: Aug. 12, 2013

Related U.S. Application Data
(60) Provisional application No. 61/407,709, filed on Oct. 28, 2010.

(51) Int. Cl. C12Q 1/68

(52) U.S. Cl. CPC C12Q 1/6886
USPC 424/174.1; 435/6.11; 435/18; 435/195; 514/19.3; 514/44 R; 514/44 A; 536/23.2

(57) ABSTRACT

The invention relates to methods and compositions for identifying mutations associated with cancer, such as melanoma. More particularly, the invention relates to methods and compositions for assessing particular mutations as markers for cancer, such as melanoma. Such methods and compositions allow medical providers to diagnose cancer, such as melanoma, based upon the presence of one or more particular mutations in a subject, as compared with an otherwise healthy control. The methods and compositions are also useful in guiding the type of therapy to be applied, and monitoring the effects of the therapy applied.

### BACKGROUND OF THE INVENTION

In 2009, the American Cancer Society predicted that there would be about 122,000 new cases of melanoma. The National Cancer Institute (NCI) Surveillance, Epidemiology and End Results (SEER) database documents a 619% increase in the annual incidence of melanoma and a 165% increase in the annual mortality from 1950 to 2000. To date, there are limited therapy options that improve the overall survival of patients with stage 1V melanoma. The median survival for stage 1V melanoma patients is approximately 9 months. Furthermore, clinically, there is no generally accepted standard care for patients with metastatic melanoma. The range of treatment options for those with melanoma includes observation, surgical resections of limited metastatic disease, palliative resections for local control, therapy with dacarbazine or temozolomide, combination chemotherapy, or immunotherapy (Herlyn et al., 2007, Semin Oncol, 34: 566).

Recent studies have suggested that cancerous tumors are highly heterogeneous and that treatment guided by tumor genotype, epigenotype, and gene expression profile may improve outcome. The implementation of this integrative approach is crucial for the treatment of melanomas, a disease that is known to be composed of different classes, as revealed by multiple approaches (John et al., 2008, Clin Cancer Res, 14:5173; Smalley et al., 2008, Cancer Res, 68:5743, Winnepenninckx et al., 2006, J Natl Cancer Inst, 98:472; Viros et al., 2008, PLoS Med, 5:e120). Genomic analysis of melanomas show that these tumors can contain mutations that induce the malignant phenotype, such as in BRAF, CDKN2A, PTEN, CTNNB1, NRAS, PIK3CA and KIT (Chin et al., 2006, Genes Dev, 20:2149; Curtin et al., 2006, J Invest Dermatol, 126:1660). However, except for BRAF (−50%) and common loss of CDKN2A, these mutations are relatively rare, existing in only a small set of melanomas (10% or less). Nevertheless, mutation analysis of tumors is already a part of clinical operating procedures, including the selection of patients for treatment with PLX4032 (Plexxikon Inc.) and Imatinib (Gleevec, Novartis Pharmaceuticals), inhibitors specific for activated BRAF and KIT, respectively (Halaban et al., 2009, PLoS ONE, 4: e4563).

Identification of mutations in receptor and non-receptor protein kinases in cancer cells that drive malignant transformation (‘driver’ mutations) have revolutionized patient care by opening the door to patient-tailored targeted therapies that improve survival and extend patients' life spans. One outstanding example is the activating V600E/K mutations in the protein kinase BRAF present in ˜40% of melanoma tumors and PLX4032/RG7204 (Plexxikon, Inc.), the BRAF-V600E/K-inhibitor (Flaherty et al., 2010, N Engl J Med 363:809). However, the abundance of BRAF-V600E/K mutations in melanomas is unique. The picture that emerges from whole exome sequencing is the presence of several kinase genes that harbor a significant number of distinct mutations that might be targeted for therapy. Studying these genes in a large cohort of melanoma may identify subgroups of patients that can benefit from current or future therapies.

Protein kinases play essential roles in signal transduction pathways that are critical for cell proliferation and migration. Some have been reported already to be ‘cancer’ genes, and for some, targeted therapies already exist. However, there is a potential for additional kinases to be implicated in the pathogenesis of tumors. Genomic sequencing can bear out novel mutations or single nucleotide variations (SNVs) that are associated with the development of tumors, including melanomas. Further, it is unknown whether these potential mutations or SNVs, which may correlate with tumors, are somatic or inherited variations.

Epidemiological observations and investigations using genetically altered mice have suggested that ultraviolet (UV) exposure can enhance melanoma development. However, there is limited understanding of the molecular pathways that are induced by UV radiation (UVR). Furthermore, the effect of UVR on melanocytes of different pigmentation, and the relationship between pigmentation and UVR effects remains to be explored (Herlyn et al., 2007, Semin Oncol, 34:566). However, interestingly it has been recently demonstrated that common genetic mutations in melanomas may in fact be correlated to UV or sun damage (Lee et al., 2011, Br J Dermatol, 164: 776; Edlundh-Rose et al., 2006, Melanoma Res, 16: 471; Bauer et al., 2011, Pigment Cell Melanoma Res, 24: 345)

Despite the advances made in the genetic classifications of cancers, such as melanoma, there is a need in the art for the further discovery of novel mutations that can drive the development and selection of targeted therapies to treat cancer, such as melanoma. The present invention fulfills these needs,

### SUMMARY OF THE INVENTION

The present invention relates to the discovery that particular somatic mutations are associated with cancer, such as melanoma. The invention relates to compositions and methods useful for the assessment, characterization and treatment of cancer, including melanoma, based upon the presence or absence of a particular somatic mutation that is associated with cancer, including melanoma. The compositions of the invention relate to the somatic mutations associated with cancer, genes having at least one somatic mutation associated with cancer, and modulators of mutant genes and wild-type genes identified by having at least one somatic mutation associated with cancer, including upstream modulators and downstream effectors of mutant genes and wild-type genes identified by having a somatic mutation associated with cancer.

The methods of the invention relate to methods of diagnosing cancer, such as melanoma, associated with a somatic mutation, methods of prognosis of cancer, such as melanoma, associated with a somatic mutation, methods of grading of cancer, such as melanoma, associated with a somatic mutation, methods of determining the optimal treatment regimen for cancer, such as melanoma, associated with a somatic mutation, and methods of monitoring the effectiveness of a treatment regimen of cancer, such as melanoma, associated with a somatic mutation. In various embodiments, the somatic mutations associated with cancer, such as melanoma, of the invention are driver mutations. The mutation useful in the compositions and methods of the invention described herein can be any mutation in any gene associated with cancer, such as melanoma. The mutation useful in the compositions and methods of the invention described herein can be any mutation in any gene elsewhere disclosed herein. The mutation useful in the compositions and methods of the invention described herein can be any mutation elsewhere described herein.

In one embodiment, the invention is an isolated RAC1 polypeptide having an amino acid sequence with at least one mutation at amino acid position 29. In a particular embodiment, the isolated RAC1 polypeptide has at least one mutation at position 29 that replaces P with S. In another embodiment, the invention is an isolated RAC1 polypeptide having an amino acid sequence with at least one mutation at amino acid position 65. In a particular embodiment, the isolated RAC1 polypeptide has at least one mutation at position 65 the replaces D with N.

In one embodiment, the invention is an isolated RAC1 nucleic acid having a nucleotide sequence with at least one mutation, where the nucleic acid encodes a polypeptide having an amino acid sequence having at least one mutation at amino acid position 29. In a particular embodiment, the isolated RAC1 nucleic acid encodes a polypeptide having a mutation at position 29 that replaces P with S.

In another embodiment, the invention is an isolated RAC1 nucleic acid having a nucleotide sequence comprising at least one mutation, where the nucleic acid encodes a polypeptide having an amino acid sequence having at least one mutation at amino acid position 65. In a particular embodiment, the isolated RAC1 nucleic acid encodes a polypeptide having a mutation at position 65 that replaces D with N.

In one embodiment, the invention is a method of identifying a mutant RAC1 sequence in a biological sample obtained from a subject, involving the steps of obtaining a biological sample from the subject, determining the sequence of the subject's RAC1 sequence, comparing the subject's RAC1 sequence to a wild-type RAC1 sequence, and identifying at least one mutation in the subject's RAC1 sequence as compared with the wild-type RAC1 sequence. The RAC1 sequence can be a nucleic acid sequence or an amino acid sequence and the mutation identified can be any mutation described elsewhere herein. In a preferred embodiment, the subject is a human.

In another embodiment, the invention is a method of diagnosing melanoma in a subject, involving the steps of obtaining a biological sample from the subject, determining the sequence of the subject's RAC1 sequence, comparing the subject's RAC1 sequence to a wild-type RAC1 sequence, and identifying at least one mutation in the subject's RAC1 sequence as compared with the wild-type RAC1 sequence. The RAC1 sequence can be a nucleic acid sequence or an amino acid sequence and the mutation identified can be any mutation described elsewhere herein. In a preferred embodiment, the subject is a human.

In a further embodiment, the invention is a method of prognosing the outcome of melanoma a subject, involving the steps of obtaining a biological sample from the subject, determining the sequence of the subject's RAC1 sequence, comparing the subject's RAC1 sequence to a wild-type RAC1 sequence, and identifying at least one mutation in the subject's RAC1 sequence as compared with the wild-type RAC1 sequence. The RAC1 sequence can be a nucleic acid sequence or an amino acid sequence and the mutation identified can be any mutation described elsewhere herein. In a preferred embodiment, the subject is a human.

In another embodiment, the invention is a method of determining the appropriate melanoma treatment regimen to administer to a subject, involving the steps of obtaining a biological sample from the subject, determining the sequence of the subject's RAC1 sequence, comparing the subject's RAC1 sequence to a wild-type RAC1 sequence, identifying at least one mutation in the subject's RAC1 sequence as compared with the wild-type RAC1 sequence, and selecting the appropriate melanoma treatment regimen to administer to the subject based upon the presence or absence of the mutation or mutations identified. The RAC1 sequence can be a nucleic acid sequence or an amino acid sequence and the mutation identified can be any mutation described elsewhere herein. In a preferred embodiment, the subject is a human.

In some embodiments, the invention is a method of preventing or of treating melanoma in a subject in need thereof, the method involving the steps of administering to the subject, a therapeutically effective amount of an inhibitor of a gene associated with melanoma, where the subject has been diagnosed as having melanoma or is at risk of developing melanoma. In various embodiments, the inhibitor is a chemical compound, a protein, a peptide, a peptidomemetic, an antibody, a ribozyme, a small molecule chemical compound, and an antisense nucleic acid molecule, or combinations thereof. In some embodiments, the gene is RAC1, a modulator of RAC1, an effector of RAC1, or a combination thereof. In other embodiments the gene is RAC1 P29X, RAC1 P29S, RAC1 D65X, or RAC1 D65N, a modulator of RAC1 P29X, RAC1 P29S, RAC1 D65X, or RAC1 D65N, an effector of RAC1 P29X, RAC1 P29S, RAC1 D65X, or RAC1 D65N, or a combination thereof. In a preferred embodiment, the subject is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A and 1B, shows the UV signature mutation and correlation to sun damage. FIG. 1A shows the spectrum of somatic variants in non-sun-exposed (top) and sun-exposed (bottom) melanomas. There is an excess of C→T transitions in the dipyrimidine context in sun-exposed melanomas, an indication of UV exposure and DNA damage for those melanomas or their precursors. FIG. 1B shows a representative primary melanoma (arrows) showing extensive solar elastosis (marked with broken lines). Scale bar represents 0.1 mm.

FIG. 2, comprising FIGS. 2A and 2B, shows the RAC1-P29S UV signature mutation. FIG. 2A shows the Sanger electropherogram showing a representative germline DNA (PBL) compared to tumor (1.1 mm primary melanoma) at the site of the RAC1-P29S mutation. FIG. 2B shows the frequency distribution of melanomas by location and sex in the cohort. The data represent 312 patients composed of 144 males and 87 females. Fisher's exact test shows that the head and neck lesions are particularly significantly enriched in men compared to women with p values as follow: Head and neck: 0.0043; Trunk: 0.097; Legs and arms: 0.055; Acral: 1.0; Ocular: 0.49.

FIG. 3 shows the alignment of human RHO-family GTPases. The switch I loop region is shown. The conserved proline corresponding to codon 29 in RAC1 is highlighted. The Swiss-Prot ID for each protein is indicated. Representative non-RHO-family GTPases are shown. Conserved residues of the G1, G2 and G3 elements are also highlighted (Wennerberg et al., 2005, J Cell Sci, 118: 843). Alignment made using ClustalW. Secondary structure elements for RAC1-P29S crystal indicated; α-helix as a cylinder, β-strand as rectangle, loop as line.

FIG. 4, comprising FIGS. 4A through 4D, shows the analysis of the Switch I region of RAC1. FIG. 4A shows lattices of RAC1-P29S and RAC1-WT crystals. Asymmetric unit colored white. The RAC1-P29S and RAC1-WT crystals pack in a very similar fashion. FIG. 4B shows a stereoview of 2Fo-Fc electron density for the switch I region of RAC1-P29S contoured at 1σ and 2σ. For clarity, electron density is clipped at 2 Å from either GMP-PNP or the switch I region. FIG. 4C shows a stereoview of 2Fo-Fc electron density for the switch I region of RAC1-WT. The switch I regions of both molecules (A and B) of the asymmetric unit are shown. The switch I loop shows poor electron density in molecule A and is not visible in molecule B. The wild-type crystal structure clearly shows that the switch I region of RAC1-WT is conformationally flexible and that this is not due to crystal packing effects. Maps for molecule A are contoured and clipped as per panel B. Maps for molecule B are contoured as per panel B and are clipped at 20 Å from GMP-PNP. FIG. 4D shows Ligplot diagrams (Wallace et al., 1995, Protein Eng, 8: 127) for GMP-PNP bound to molecules A (left) and B (right) of the P2₁2₁2₁ crystal structure of RAC1-P29S.

FIG. 5, comprising FIGS. 5A through 5E, shows the crystal structure of RAC1-P29S. FIG. 5A shows the overall schematic of RAC1-P29S showing the P-loop, switch I, switch II, Mg²⁺ and the slowly hydrolyzing GTP analogue, GMP-PNP (stick format). The sphere indicates the location of P29S. Dashed box indicates region shown in FIG. 5B to FIG. 5E. FIG. 5B shows a close-up view showing close contacts of ribose hydroxyls with the switch I region in RAC1-P29S. FIG. 5C shows RAC1-WT bound to GMP-PNP and FIG. 5D shows HRAS in complex with GMP-PNP, PDB ID: 5P21 (Pai et al., 1990, Embo J, 9: 2351). Direct hydrogen bonding between the switch I backbone and ribose are commonly observed in activated HRAS, but less frequently observed in RHO-family GTPases including RAC1. A RAS-like hydrogen bonding pattern is observed in activated RAC1-P29S (dashed lines). FIG. 5E shows the superposition of switch I region and GMP-PNP, with RAC1-P29S, RAC1-WT and HRAS. The sphere indicates the location of P29S. Residues discussed are labeled. Figure made using CCP4 mg (Potterton et al., 2004, Acta Crystallog D Biol Crystallogr, 60:2288).

FIG. 6, comprising FIGS. 6A through 6C, shows switch I conformations. FIG. 6A shows a comparison of carbon-alpha trace for representative GTP-bound, or GTP-analogue-bound, GTPase crystal structures superposed onto the crystal structures of RAC1-P29S, RAC1-WT, RHOA1 (1A2B), RAB3A (3RAB), RAS (5P21) and RAN (1RRP). PDB ID in parentheses. Location of RAC1 P29S is shown as a sphere. FIG. 6B shows the superposition of representative GTP-bound, or GTP-analogue bound, RHO family GTPases onto the crystal structure of RAC1-P29S (sphere) showing that for the switch I loop of RAC1-P29S is conformationally divergent. Crystal structures of each of, 1A2B, 1AM4, 1CEE, 1CXZ, 1E0A, 1GWN, 1I4T, 1KMQ, 1M7B, 1NF3, 1RYH, 1S1C, 1Z2C, 2ATX, 2FJU, 2GCO, 2GCP, 2IC5, 2ODB, 2V2, 2QME, 2QRZ, 2RMK, 2V55, 2W2V, 2W2X, 2WKQ, 3EG5 and 3KZ1 are shown. RAC1P29S shown by arrow. Location of RAC1 P29S is shown as a sphere. FIG. 6C shows the superposition of representative GTP-bound, or GTP-analogue-bound, non-RHO-family GTPases onto the crystal structure of RAC1-P29S (sphere) showing that the switch I loop of RAC1-P29S adopts a similar, RAS-like, conformation. Crystal structures of each of 121P, 1AGP, 1C1Y, 1CLU, 1CTQ, 1EK0, 1G17, 1GNP, 1GNQ, 1GNR, 1GUA, 1HE8, 1HUQ, 1IAQ, 1IBR, 1JAH, 1JAI, 1K5D, 1K8R, 1KY2, 1LF0, 1LFD, 1N6H, 1N6L, 1N6N, 1N6O, 1N6P, 1N6R, 1NVU, 1NVW, 1NVX, 1OIW, 1P2S, 1P2T, 1P2U, 1P2V, 1PLJ, 1PLK, 1QBK, 1QRA, 1R2Q, 1RRP, 1RVD, 1T91, 1TU3, 1U8Y, 1UAD, 1VG0, 1X3S, 1XCM, 1XTR, 1XTS, 1YHN, 1YU9, 1YVD, 1YZK, 1YZL, 1YZN, 1YZQ, 1lYZT, 1YZU, 1Z06, 1Z07, 1Z08, 1Z0J, 1Z0K, 1ZBD, 1ZC3, 1ZC4, 1ZW6, 221P, 2BME, 2C5L, 2CL0, 2CL6, 2CL7, 2D7C, 2EVW, 2EW1, 2F9M, 2FFQ, 2FG5, 2G6B, 2GIL, 2GZD, 2GZH, 2HV8, 2OCB, 2RAP, 2RGA, 2RGB, 2RGC, 2RGD, 2RGE, 2RGG, 2UZI, 2VH5, 2X19, 2ZET, 3A6P, 3BBP, 3BC1, 3CWZ, 3DDC, 3E5H, 3GFT, 3GJX, 313S, 3K8Y, 3K9L, 3K9N, 3KKM, 3KKN, 3KKO, 3L8Y, 3L8Z, 3LAW, 3LBH, 3LBI, 3LBN, 3M1I, 3MJH, 3NBY, 3NBZ, 3NC0, 3NC0, 3NC1, 3NKV, 3OES, 3OIU, 3OIV, 3OIW, 3PIR, 3PIT, 3QBT, 3RAB, 3RAP, 3RAP, 421P, 521P, 5P21, 621P, 6Q21, 721P and 821P are shown. RAC1P29S shown by arrow. Location of RAC1 P29S is shown as a sphere. Figure made using Pymol (www.pymol.org).

FIG. 7, comprising FIGS. 7A through 7F, shows that RAC1-P29S is more proficient in effector binding, accelerates normal melanocyte proliferation and migration, and accumulates in ruffle membranes. FIG. 7A shows an in vitro PAK1 pull-down assay showing that recombinant His-RAC1-P29S displays an increased binding to its effector PAK1 compared to His-RAC1-WT for each of the samples that include GTP, GMP-PNP and GTPγS, NC, PAK1-PBD beads alone as negative control. His-RAC1-WT and His-RAC1-P29S lanes show unbound wild type and mutant protein, as indicated. The multiple bands in all lanes are RAC1, because the crystallization-quality His-RAC1-WT and His-RAC1-P29S used in the assay migrate in the gel in a fashion similar to that of PAK1-PBD bound proteins. FIG. 7B shows a western blot analysis of RAC1 in murine melanocytes infected with wild type (WT) or mutant RAC1-P29S retroviral expression vectors, compared to non-transfected cells (parental). Actin indicates protein loading in each lane. FIG. 7C shows that RAC1-P29S enhances melanocyte proliferation. The proliferation of melanocytes transiently expressing RAC1 wild type (WT, square) or RAC1-P29S (circle) is compared to non-infected parental cells (triangle). Symbols are as in B. Bars indicate standard error. FIG. 7D shows that RAC1-P29S enhances cell migration. The rate of migration of parental, RAC1-WT and RAC1-P29S expressing normal melanocytes was determined using the Cultrex 24-well cell migration assay (Trevigen Cell Assay, Trevigen Inc., CT) at daily intervals (Halaban et al., 2010, Pigment Cell Melanoma Res, 23: 190). FIG. 7E shows that RAC1-P29S enhances ERK activation. Western blots of lysates from normal mouse melanocytes expressing RAC1 wild type (WT) or RAC1-P29S transgene probed with antibodies as indicated. FIG. 7F shows the localization of GFP-tagged RAC1-WT (a-b) and RAC1-P29S (c-d) in COS-7 cells. Transiently expressing GFP-tagged RAC1-WT (a-b), or RAC1-P29S (c-d) COS-7 cells were fixed with paraformaldehyde and imaged with a spinning-disk confocal based inverted Olympus microscope. Insets (boxes) illustrate localization of RAC1-P29S, but not RAC1-WT, in membrane ruffles (compare a-b to c-d, arrows, respectively). Scale Bars represent 10mM.

FIG. 8 depicts the structure of autoinhibited Src kinase showing mutations in SRMS, BLK and FGR. The structure of autoinhibited Src kinase is shown, with SH3, SH2, and kinase domains labeled. The linker between the SH2 and kinase domains is labeled ‘Linker,’ the C-terminal tail containing pY527 is labeled ‘Tail’ and the ATP-binding cleft is labeled “ATP-cleft. Spheres mark the predicted locations of mutations in SRMS, BLK and FGR. Clusters of mutations are labeled.

FIG. 9 is a schematic depicting the identified PAK family member mutations in melanomas. The locations of the p21-binding and autoregulation domains (PBD:AID), also known as the CRIB, or CDC42/Rac interactive binding domain, kinase domain, and protein-protein interaction sites (proline-rich regions, GID, PIX binding site) are noted in the figure. Mutations in melanomas were found in the kinase domains of PAK-4 and PAK5/7 and in the interactive CDC42/Rac binding domain (PBD) of PAK2, PAK-4 and PAK6, and thus may have impact on the function of the protein.

FIG. 10 is a schematic of the TNK2 gene coding for the ACK1 (NM_{—}005781.4) functional domains and sites of missense mutations identified in cancer cells (Chua et al., 2010, Mol Oncol, 2010:323-34; Galsteo et al., 2006, Proc Natl Acad Sci USA, 103:9796-9801). S212L, S245F and S522I are mutations in melanoma cells identified by studies described herein. The P731L SNV was found in the healthy population database.

FIG. 11 is a table detailing the characteristics of samples sequence by exome capture.

FIG. 12 is a table providing a summary of the clinical, pathological, and mutation status of melanoma samples.

FIG. 13, comprising FIGS. 13A and 13B, is a table listing recurrent coding somatic SNVs identified with the discovery and validation screens.

FIG. 14, comprising FIGS. 14A and 14B, provides patient information about the RAC1 P29S mutation. FIG. 14A details clinical and genetic information of melanomas with the RAC1 P29S mutation. FIG. 14B shows the distribution of RAC1 P29S in the studied cohort.

FIG. 15 is a table detailing the RAC1 crystal structure data collection and refinement statistics.

FIG. 16, comprising FIGS. 16A-16E, is a table listing novel SNVs in RAC1 regulators and signaling genes.

FIG. 17, comprising FIG. 17A-17B, is a table showing the count of novel SNVs in RAC1 regulator and signaling genes.

FIG. 18 is a table detailing selected novel somatic mutations in genes encoding tyrosine kinases, Ser/Thr kinases and kinase regulatory proteins in melanomas.

FIG. 19, comprising FIG. 19A-19J, is a table showing genes identified having a somatic or inherited mutation.

FIG. 20 is a table detailing the characteristics of melanomas having an ACK1 mutation.

FIG. 21 is a schematic depicting the modulators and effectors in the RAC1 signaling pathway.

FIG. 22, comprising FIGS. 22A-22H, is a table showing mutations identified.

### DETAILED DESCRIPTION OF THE INVENTION

The present invention relates to the discovery that particular somatic mutations are associated with cancer, such as melanoma. The invention relates to compositions and methods useful for the assessment, characterization and treatment of cancer, including melanoma, based upon the presence or absence of a particular somatic mutation that is associated with cancer, including melanoma. The compositions of the invention relate to the somatic mutations associated with cancer, genes having at least one somatic mutation associated with cancer, and modulators of genes identified by having at least one somatic mutation associated with cancer, as well as modulators of genes upstream and downstream of the genes identified by having a somatic mutation associated with cancer. The methods of the invention relate to methods of diagnosing cancer, such as melanoma, associated with a somatic mutation, methods of prognosis of cancer, such as melanoma, associated with a somatic mutation, methods of grading of cancer, such as melanoma, associated with a somatic mutation, methods of determining the optimal treatment regimen for cancer, such as melanoma, associated with a somatic mutation, and methods of monitoring the effectiveness of a treatment regimen of cancer, such as melanoma, associated with a somatic mutation. In various embodiments, the somatic mutations associated with cancer, such as melanoma, of the invention are driver mutations.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variants”, “polymorphisms”, or “mutations.”

As used herein, to “alleviate” a disease means reducing the frequency or severity of at least one sign or symptom of a disease or disorder.

As used herein the terms “alteration,” “defect,” “variation,” or “mutation,” refers to a mutation in a gene in a melanoma cell that affects the function, activity, expression (transcription or translation) or conformation of the polypeptide that it encodes. Mutations encompassed by the present invention can be any mutation of a gene in a melanoma cell that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing the mRNA (splice site mutation) or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “applicator,” as the term is used herein, is meant any device including, but not limited to, a hypodermic syringe, a pipette, an iontophoresis device, a patch, and the like, for administering the compositions of the invention to a subject.

The term “auto-antigen” means, in accordance with the present invention, any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “cancer,” has used herein includes, but is not limited to, cancers of the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, melanoma, etc.), breast, genital system, (e.g., uterus, ovary, prostate, testis, etc.), urinary system (e.g, bladder, kidney, ureter, etc.), eye, nervous system (e.g., brain, etc.), endocrine system (e.g., thyroid, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the term “control nucleic acid” is meant to refer to a nucleic acid (e.g., RNA, DNA) that does not come from a subject known to have, or suspected to have, a mutation in a gene in a melanoma cell (e.g., for a control subject). For example, the control can be a wild type nucleic acid sequence which does not contain a variation in its nucleic acid sequence. Also, as used herein, a control nucleic acid can be a fragment or portion of gene that does not include the defect/variation that is the mutation of interest (that is, the mutation to be detected in an assay).

As used herein, “clonal expansion” refers to the increase in the number of progeny cells originating from one cell.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “diagnosis” refers to the determination of the nature of a case of disease. In some embodiments of the present invention, methods for making a diagnosis are provided which permit determination of a particular mutation associated with cancer, such as melanoma.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By “driver mutation,” as used herein, is meant a mutation that is causally associated with the development, survival, proliferation and/or the progression of a cancer cell. Typically, a driver mutation imparts a growth advantage on the cancer cell. A driver mutation is often, but is not necessarily, required for the continued maintenance of the cancer cell phenotype. In contrast, a “passenger mutation” is a mutation present in a cancer cell genome that is present in the cancer cell genome, and does not functionally contribute to the cancer cell phenotype.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “housekeeping gene” as used herein refers to genes that are generally always expressed and thought to be involved in routine cellular metabolism. Housekeeping genes are well known and include such genes as glyceraldehyde-3-phosphate dehydrogenase (G3PDH or GAPDH), albumin, actins, tubulins, cyclophilin, hypoxanthine phsophoribosyltransferase (HRPT), 28S, and 18S rRNAs and the like.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “microarray” and “array” refers broadly to both “DNA microarrays” and “DNA chip(s),” and encompasses all art-recognized solid supports, and all art-recognized methods for affixing nucleic acid molecules thereto or for synthesis of nucleic acids thereon. Preferred arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, Arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant” as used herein, refers to either a nucleic acid or protein comprising a mutation.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

The term “perfect match,” “match,” “perfect match probe” or “perfect match control” refers to a nucleic acid that has a sequence that is perfectly complementary to a particular target sequence. The nucleic acid is typically perfectly complementary to a portion (subsequence) of the target sequence. A perfect match (PM) probe can be a “test probe”, a “normalization control” probe, an expression level control probe and the like. A perfect match control or perfect match is, however, distinguished from a “mismatch” or “mismatch probe.” The term “mismatch,” “mismatch control” or “mismatch probe” refers to a nucleic acid whose sequence is not perfectly complementary to a particular target sequence. As a non-limiting example, for each mismatch (MM) control in a high-density probe array there typically exists a corresponding perfect match (PM) probe that is perfectly complementary to the same particular target sequence. The mismatch may comprise one or more bases. While the mismatch(es) may be located anywhere in the mismatch probe, terminal mismatches are less desirable because a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “reaction mixture” or “PCR reaction mixture” or “master mix” or “master mixture” refers to an aqueous solution of constituents in a PCR reaction that can be constant across different reactions. An exemplary PCR reaction mixture includes buffer, a mixture of deoxyribonucleoside triphosphates, primers, probes, and DNA polymerase. Generally, template RNA or DNA is the variable in a PCR.

“Sample” or “biological sample” as used herein means a biological material isolated from a subject. The biological sample may contain any biological material suitable for detecting a mutation in a gene in melanoma, and may comprise cellular and/or non-cellular material obtained from the individual.

A “somatic mutation,” as used herein, is a genetic alteration acquired by a somatic cell that can be passed on to progeny cells of the mutated somatic cell in the course of cell division. Somatic mutations differ from germ line mutations, which are inherited genetic alterations that occur in germ cells.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, “substantially purified” refers to being essentially free of other components. For example, a substantially purified cell is a cell which has been separated from other cell types with which it is normally associated in its naturally occurring state. In some instances, a population of substantially purified cells refers to a homogenous population of cells. In other instances, this term refers simply to a cell that have been separated from the cells with which they are naturally associated in their natural state.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Targets are sometimes referred to in the art as anti-probes. As the term targets is used herein, no difference in meaning is intended.

As used herein, the terms “therapy” or “therapeutic regimen” refer to those medical steps taken to alleviate or alter a disorder or disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disorder or disease state but in many instances the effect of a therapy will have non-desirable or side-effects. The effect of therapy will also be impacted by the physiological state of the host, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

### Description

The present invention relates to the discovery that particular somatic mutations are associated with cancer, such as melanoma. The invention relates to compositions and methods useful for the assessment, characterization and treatment of cancer, such as melanoma, based upon the presence or absence of particular somatic mutations that are associated with cancer, such as melanoma.

In one embodiment, the compositions of the invention relate to somatic mutations associated with melanoma, genes having at least one somatic mutation associated with melanoma, and modulators of genes identified by having at least one somatic mutation associated with melanoma, as well as modulators of genes upstream and downstream of the genes identified by having a somatic mutation associated with melanoma.

In another embodiment, the methods of the invention relate to methods of diagnosing melanoma associated with a somatic mutation, methods of prognosis of melanoma associated with a somatic mutation, methods of grading of melanoma associated with a somatic mutation, methods of determining the optimal treatment regimen for melanoma associated with a somatic mutation, and methods of monitoring the outcome of a treatment regimen of melanoma associated with a somatic mutation.

In various embodiments, the somatic mutation associated with melanoma of the invention is a driver mutation. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

### Assays

The present invention relates to the discovery that particular mutations are associated with the development and progression of cancer, such as melanoma. In various embodiments, the invention relates to a genetic screening assay of a subject to determine whether the subject has a mutation associated with cancer, such as melanoma. The present invention provides methods of assessing for the presence or absence of a mutation associated with cancer, such as melanoma, as well as methods of diagnosing a subject having a mutation associated with cancer, such as melanoma. The mutations associated with cancer described herein include alterations (e.g., substitution, deletion, insertion, or transition) in the nucleic acid sequence of a variety of genes, as elsewhere described herein throughout. The positions of the mutations in the gene sequences described herein are numbered in relation to the nucleic acid sequence or amino acid sequence. That is, the numbered position of an altered nucleotide, or amino acid, is the position number of that nucleotide, or amino acid, in the nucleic acid or amino acid sequence. In various embodiments, the somatic mutations associated with cancer, such as melanoma, of the invention are driver mutations. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

In one embodiment, the method of the invention is a diagnostic assay for diagnosing melanoma in a subject in need thereof, by determining whether a mutation is present in a gene associated with melanoma in a biological sample obtained from the subject. The results of the diagnostic assay can be used alone, or in combination with other information from the subject, or from the biological sample obtained from the subject. The mutation identified by the assay can be any mutation in any gene associated with melanoma. The mutation identified by the assay can be any mutation in any gene elsewhere disclosed herein. The mutation identified by the assay can be any mutation elsewhere described herein. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

In another embodiment, the method of the invention is a prognostic assay for developing a prognosis of the outcome of melanoma in a subject in need thereof, by determining whether a mutation is present in a gene associated with melanoma in a biological sample obtained from the subject. The results of the prognostic assay can be used alone, or in combination with other information from the subject, or from the biological sample obtained from the subject. The mutation identified by the assay can be any mutation in any gene associated with melanoma. The mutation identified by the assay can be any mutation in any gene elsewhere disclosed herein. The mutation identified by the assay can be any mutation elsewhere described herein. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

In a further embodiment, the method of the invention is an assay for monitoring the effectiveness of a treatment for melanoma in a subject in need thereof, by determining whether a mutation is present in a gene associated with melanoma in a biological sample obtained from the subject. The assay can be performed before, during or after the treatment has been administered, or any combination thereof. The results of the assay can be used alone, or in combination with other information from the subject, or from the biological sample obtained from the subject. The mutation identified by the assay can be any mutation in any gene associated with melanoma. The mutation identified by the assay can be any mutation in any gene elsewhere disclosed herein. The mutation identified by the assay can be any mutation elsewhere described herein. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

In another embodiment, the method of the invention is an assay for determining the appropriate treatment for melanoma to be administered to subject in need thereof, by determining whether a particular mutation is present in a particular gene associated with melanoma in a biological sample obtained from the subject. Certain driver mutations associated with melanoma are known to respond better to certain treatment regimens, and less well, or not all, to other treatment regimens. Thus, the mutations identified in the assays of the invention are useful in determining the appropriate treatment regimen to administer to a particular subject. Knowing which particular mutation, or mutations, are present in the biological sample of a particular subject, can guide the medical provider to select the optimal treatment regimen (e.g., the drug, the does, the frequency of administration) to administer to any particular subject. The assay can be performed, before, during or after the treatment has been administered, or any combination thereof. The results of the assay can be used alone, or in combination with other information from the subject, or the biological sample obtained from the subject. The mutation identified by the assay can be any mutation in any gene associated with melanoma. The mutation identified by the assay can be any mutation in any gene elsewhere disclosed herein. The mutation identified by the assay can be any mutation elsewhere described herein. In one embodiment, the gene associated with melanoma is RAC1. In some embodiments, the mutant gene associated melanoma is RAC1 P29X, which is a RAC1 gene comprising at least one mutation at position 29 where P is replaced by another amino acid residue. In one embodiment, the mutant gene associated with melanoma is RAC1 P29S, which is a RAC1 gene comprising at least one mutation at position 29 where P is replaced by S. In other embodiments, the mutant gene associated melanoma is RAC1 D65X, which is a RAC1 gene comprising at least one mutation at position 65 where D is replaced by another amino acid residue. In one embodiment, the mutant gene associated with melanoma is RAC1 D65N, which is a RAC1 gene comprising at least one mutation at position 65 where D is replaced by N.

In the assay methods of the invention, a test biological sample from a subject is assessed for the presence of at least one mutation in at least one gene associated with melanoma. In various embodiments, the subject is a human subject, and may be of any race, sex and age. Representative subjects include those who are suspected of having melanoma, those who have been diagnosed with melanoma, those whose have melanoma, those who have had melanoma, those who at risk of a recurrence of melanoma, and those who are at risk of developing melanoma.

In one embodiment, the test sample is a sample containing at least a fragment of a nucleic acid of a gene, or mutant gene, associated with melanoma. The term, “fragment,” as used herein, indicates that the portion of the gene, DNA, mRNA or cDNA is a polynucleotide of a length that is sufficient to identify it as a fragment of a gene, or mutant gene, associated with melanoma. In one representative embodiment, a fragment comprises one or more exons of the gene, or mutant gene, associated with melanoma. In another representative embodiment, a fragment comprises part of an exon of the gene, or mutant gene, associated with melanoma. In some embodiments, the fragment can also include an intron/exon junction of the gene, or mutant gene, associated with melanoma.

The test sample is prepared from a biological sample obtained from the subject. The biological sample can be a sample from any source which contains nucleic acid (e.g., DNA, chromosomal nucleic acid, or RNA), such as a body fluid or a tissue, or a tumor, or a combination thereof A biological sample of nucleic acid from a cell, or melanoma cell, or a tumor can be obtained by appropriate methods, such as, for example, biopsy. In certain embodiments, a biological sample containing genomic DNA is used. A biological sample can be used as the test sample; alternatively, a biological sample can be processed to enhance access to nucleic acids, or copies of nucleic acids (e.g., copies of nucleic acids comprising a mutation associated with melanoma), and the processed biological sample can then be used as the test sample. For example, in various embodiments, nucleic acid (e.g., genomic DNA or cDNA prepared from mRNA) is prepared from a biological sample, for use in the methods. Alternatively or in addition, if desired, an amplification method can be used to amplify nucleic acids comprising all or a fragment of a mRNA or genomic DNA in a biological sample, for use as the test sample in the assessment for the presence or absence of a mutation associated with melanoma.

The test sample is assessed to determine whether one or more mutations are present in the nucleic acid of the subject. In general, detecting a mutation may be carried out by determining the presence or absence of nucleic acids containing a mutation of interest in the test sample.

In some embodiments, hybridization methods, such as Southern analysis, Northern analysis, or in situ hybridizations, can be used (see Current Protocols in Molecular Biology, Ausubel, F. et al., eds., John Wiley & Sons, including all supplements). For example, the presence of a mutation can be indicated by hybridization of nucleic acid in the genomic DNA, RNA, or cDNA to a nucleic acid probe. A “nucleic acid probe”, as used herein, can be a DNA probe or an RNA probe; the nucleic acid probe can contain at least one polymorphism of interest, as described herein. The probe can be, for example, the gene, a gene fragment (e.g., one or more exons), a vector comprising the gene, a probe or primer, etc. For representative examples of use of nucleic acid probes, see, for example, U.S. Pat. Nos. 5,288,611 and 4,851,330.

To detect one or more mutations of interest, a hybridization sample is formed by contacting the test sample with at least one nucleic acid probe. A preferred probe for detecting mRNA or genomic DNA is a labeled nucleic acid probe capable of hybridizing to mRNA or genomic DNA of a gene, or a mutant gene, associated with melanoma. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA, cDNA or genomic DNA. The hybridization sample is maintained under conditions which are sufficient to allow specific hybridization of the nucleic acid probe to mRNA, cDNA or genomic DNA of a gene, or mutant gene, associated with melanoma. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a mutant sequence and a gene, mRNA or cDNA in the test sample, the mutation that is present in the nucleic acid probe is also present in the nucleic acid sequence of the subject. More than one nucleic acid probe can also be used concurrently in this method. Specific hybridization of any one of the nucleic acid probes is indicative of the presence of the mutation of interest, as described herein.

In Northern analysis (see Current Protocols in Molecular Biology, Ausubel, F. et al., eds., John Wiley & Sons, supra), the hybridization methods described above are used to identify the presence of a mutation of interest in an RNA, such as a mRNA. For Northern analysis, a test sample comprising RNA is prepared from a biological sample from the subject by appropriate means. Specific hybridization of a nucleic acid probe, as described above, to RNA from the subject is indicative of the presence of a mutation of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising one or more mutations of interest. Hybridization of the PNA probe to a nucleic acid sequence is indicative of the presence of the mutation of interest.

In another embodiment of the methods of the invention, mutation analysis by restriction digestion can be used to detect a gene, or mutant gene, associated with melanoma, if the mutation results in the creation or elimination of a restriction site. A sample containing nucleic acid from the subject is used. Polymerase chain reaction (PCR) can be used to amplify all or a fragment of a nucleic acid (and, if necessary, the flanking sequences) in the sample. RFLP analysis is conducted as described (see Current Protocols in Molecular Biology, supra). The digestion pattern of the relevant fragments indicates the presence or absence of mutation in gene, or mutant gene, associated with melanoma.

Direct sequence analysis can also be used to detect specific mutations in a gene associated with melanoma. A sample comprising DNA or RNA can be used, and PCR or other appropriate methods can be used to amplify all or a fragment of the nucleic acid, and/or its flanking sequences, if desired. The sequence, or a fragment thereof (e.g., one or more exons), or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of gene of interest, as appropriate. The presence or absence of a mutation can then be identified.

Allele-specific oligonucleotides can also be used to detect the presence of a mutation in gene associated with melanoma, through, for example, the use of dot-blot hybridization of amplified oligonucleotides with allele-specific oligonucleotide (ASO) probes (see, for example, 1986, Saiki et al., Nature 324:163-166). An “allele-specific oligonucleotide” (also referred to herein as an “allele-specific oligonucleotide probe”) is an oligonucleotide of approximately 10-50 base pairs, preferably approximately 15-30 base pairs, that specifically hybridizes to the mutant sequence, and that contains a mutation. An allele-specific oligonucleotide probe that is specific for a particular mutation can be prepared, using standard methods (see Current Protocols in Molecular Biology, supra). To identify a mutation, a sample comprising nucleic acid is used. PCR can be used to amplify all or a fragment of the test nucleic acid sequence. The nucleic acid containing the amplified sequence (or fragment thereof) is dot-blotted, using standard methods (see Current Protocols in Molecular Biology, supra), and the blot is contacted with the oligonucleotide probe. The presence of specific hybridization of the probe to the amplified nucleic acid is then detected. Specific hybridization of an allele-specific oligonucleotide probe containing the mutation of interest, to test nucleic acid from the subject is indicative of the presence of the mutation of interest.

In another embodiment of the invention, fluorescence resonance energy transfer (FRET) can be used to detect the presence of a mutation. FRET is the process of a distance-dependent excited state interaction in which the emission of one fluorescent molecule is coupled to the excitation of another. A typical acceptor and donor pair for resonance energy transfer consists of 4-[[4-(dimethylamino) phenyl]azo]benzoic acid (DABCYL) and 5[(2-aminoethylamino]naphthalene sulfonic acid (EDANS). EDANS is excited by illumination with 336 nm light, and emits a photon with wavelength 490 n×n. If a DABCYL moiety is located within 20 angstroms of the EDANS, this photon will be efficiently absorbed. DABCYL and MANS will be attached to two different oligonucleotide probes designed to hybridize head-to-tail to nucleic acid adjacent to and/or overlapping the site of one of the mutations of interest. Melting curve analysis is then applied: cycles of denaturation, cooling, and re-heating are applied to a test sample mixed with the oligonucleotide probes, and the fluorescence is continuously monitored to detect a decrease in DABCYL fluorescence or an increase in EDANS fluorescence (loss of quenching). While the two probes remain hybridized adjacent to one another, FRET will be very efficient. Physical separation of the oligonucleotide probes results in inefficient FRET, as the two dyes are no longer in close proximity. The presence or absence of a mutation of interest can be assessed by comparing the fluorescence intensity profile obtained from the test sample, to fluorescence intensity profiles of control samples comprising known mutations of interest.

In another embodiment, arrays of oligonucleotide probes that are complementary to target nucleic acid sequence segments from a subject can be used to identify mutations in a gene associate with melanoma. For example, in one embodiment, an oligonucleotide array can be used. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and scanned for mutations. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein. In brief, a target nucleic acid sequence which includes one or more previously identified mutations or markers is amplified by well-known amplification techniques, e.g., PCR. Typically, this involves the use of primer sequences that are complementary to the two strands of the target sequence both upstream and downstream of the mutation. Asymmetric PCR techniques may also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although often described in terms of a single detection block (e.g., for detection of a single mutation), arrays can include multiple detection blocks, and thus be capable of analyzing multiple, specific mutations. In alternate arrangements, it will generally be understood that detection blocks may be grouped within a single array or in multiple, separate arrays so that varying, optimal conditions may be used during the hybridization of the target to the array. This allows for the separate optimization of hybridization conditions for each situation. Additional description of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect mutations of interest in genes associated with melanoma. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (1981, Sheffield et al., Proc. Natl. Acad. Sci. USA 86:232-236), mobility shift analysis (1989, Orita et al., Proc. Natl. Acad. Sci. USA 86:2766-2770; 1987, Rosenbaum and Reissner, Biophys. Chem. 265:1275; 1991, Keen et al., Trends Genet. 7:5); restriction enzyme analysis (1978, Flavell et al., Cell 15:25; 1981, Geever, et al., Proc. Natl. Acad. Sci. USA 78:5081); heteroduplex analysis; chemical mismatch cleavage (CMC) (1985, Cotton et al., Proc. Natl. Acad. Sci. USA 85:4397-4401); RNase protection assays (1985, Myers, et al., Science 230:1242); use of polypeptides which recognize nucleotide mismatches, such as E. coli mutS protein (see, for example, U.S. Pat. No. 5,459,039); Luminex xMAP™ technology; high-throughput sequencing (HTS) (2011, Gundry and Vijg, Mutat Res, doi:10.1016/j.mrfmmm 2011.10.001); next-generation sequencing (NGS) (2009, Voelkerding et al., Clinical Chemistry 55:641-658; 2011, Su et al., Expert Rev Mol. Diagn. 11:333-343; 2011, Ji and Myllykangas, Biotechnol Genet Eng Rev 27:135-158); ion semiconductor sequencing (2011, Rusk, Nature Methods doi:10.1038/nmeth.f.330; 2011, Rothberg et al., Nature 475:348-352) and/or allele-specific PCR, for example. These and other methods can be used to identify the presence of one or more mutations of interest in the genes associated with cancer, such as melanoma, in a biological sample obtained from a subject. In one embodiment of the invention, the methods of assessing a biological sample for the presence or absence of a mutation in a gene associated with melanoma, as described herein, are used to diagnose in a subject a mutation in a gene associated with melanoma. Furthermore, more than one mutation may be found.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including genomic DNA. The nucleic acid can be double-stranded or single-stranded (i.e., a sense or an antisense single strand) and can be complementary to a nucleic acid encoding a polypeptide. The nucleic acid content may also be an RNA o DNA extraction performed on a fresh or fixed tissue sample.

Routine methods also can be used to extract genomic DNA from a tissue sample, including, for example, phenol extraction. Alternatively, genomic DNA can be extracted with kits such as the QJAamp™. Tissue Kit (Qiagen, Chatsworth, Calif.), the Wizard™ Genomic DNA purification kit (Promega, Madison, Wis.), the Puregene DNA Isolation System (Gentra Systems, Inc., Minneapolis, Minn.), and the A.S.A.P.™ Genomic DNA isolation kit (Boehringer Mannheim, Indianapolis, Ind.).

There are many methods known in the art for the detection of specific nucleic acid sequences and new methods are continually reported. A great majority of the known specific nucleic acid detection methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids of exogenous organisms in a body sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, more preferably at least 25 nucleotides, having a sequence complementary to a desired region of the gene, or mutant gene, of interest is hybridized in a sample, subjected to depolymerizing conditions, and the sample is treated with an ATP/luciferase system, which will luminesce if the nucleice sequence is present. In quantitative Southern blotting, levels of the mutant gene can be compared to wild-type levels of the gene.

A further process for the detection of hybridized nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed before. Most preferably the detection of the duplex is done using at least one primer directed to a RS. In yet another embodiment of PCR, the detection of the hybridized duplex comprises electrophoretic gel separation followed by dye-based visualization.

DNA amplification procedures by PCR are well known and are described in U.S. Pat. No. 4,683,202. Briefly, the primers anneal to the target nucleic acid at sites distinct from one another and in an opposite orientation. A primer annealed to the target sequence is extended by the enzymatic action of a heat stable DNA polymerase. The extension product is then denatured from the target sequence by heating, and the process is repeated. Successive cycling of this procedure on both DNA strands provides exponential amplification of the region flanked by the primers.

Amplification is then performed using a PCR-type technique, that is to say the PCR technique or any other related technique. Two primers, complementary to the gene, or mutant gene, associated with melanoma are then added to the nucleic acid content along with a polymerase, and the polymerase amplifies the DNA region between the primers.

The expression specifically hybridizing in stringent conditions refers to a hybridizing step in the process of the invention where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the DNA under conditions of stringency that prevent non-specific binding but permit binding of this DNA which has a significant level of homology with the probe or primer.

Among the conditions of stringency is the melting temperature (Tm) for the amplification step using the set of primers, which is in the range of about 55° C. to about 70° C. Preferably, the Tm for the amplification step is in the range of about 59° C. to about 72° C. Most preferably, the Tm for the amplification step is about 60° C.

Typical hybridization and washing stringency conditions depend in part on the size (i.e., number of nucleotides in length) of the DNA or the oligonucleotide probe, the base composition and monovalent and divalent cation concentrations (Ausubel et al., 1994, eds Current Protocols in Molecular Biology).

In a preferred embodiment, the process for determining the quantitative and qualitative profile according to the present invention is characterized in that the amplifications are real-time amplifications performed using a labeled probe, preferably a labeled hydrolysis-probe, capable of specifically hybridizing in stringent conditions with a segment of a gene, or mutant gene, associated with melanoma. The labeled probe is capable of emitting a detectable signal every time each amplification cycle occurs, allowing the signal obtained for each RS rearrangement to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In another preferred embodiment, the process for determining the quantitative and qualitative profile according to the present invention is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the invention includes a primer that is complementary to a nucleic acid sequence flanking the mutation of interest, and more particularly the primer includes 12 or more contiguous nucleotides substantially complementary to the sequence flanking the mutation of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

The present invention also pertains to kits useful in the methods of the invention. Such kits comprise components useful in any of the methods described herein, including for example, hybridization probes or primers (e.g., labeled probes or primers), reagents for detection of labeled molecules, restriction enzymes (e.g., for RFLP analysis), allele-specific oligonucleotides, means for amplification of subject's nucleic acids, or means for analyzing the nucleic acid sequence of the gene of interest and instructional materials. For example, in one embodiment, the kit comprises components useful for analysis of mutations associated with melanoma. In a preferred embodiment of the invention, the kit comprises components for detecting one or more of the mutations of associated with melanoma elsewhere described herein.

### Therapeutic Compositions and Methods

In various embodiments, the present invention includes compositions and methods of treating pathologies associated with cancer, such as melanoma, by diminishing the expression level, or activity level, of a gene, or mutant gene, the expression of which gene is associated with cancer, such as melanoma. It will be understood by one skilled in the art, based upon the disclosure provided herein, that a decrease in the level of a gene, or mutant gene, associated with cancer, such as melanoma, encompasses the decrease in expression of the gene, or mutant gene, associated with cancer, such as melanoma. Additionally, the skilled artisan will appreciate, once armed with the teachings of the present invention, that a decrease in the level of a gene, or mutant gene, associated with cancer, such as melanoma, includes a decrease in the activity of a gene, or mutant gene, associated with cancer, such as melanoma. Thus, decreasing the level or activity of gene, or mutant gene, the expression of which gene is associated with cancer, such as melanoma, includes, but is not limited to, decreasing transcription, translation, or both, of a nucleic acid encoding a gene, or mutant gene, associated with cancer, such as melanoma; and it also includes decreasing any activity of the gene, or mutant gene, associated with cancer, such as melanoma, as well. In some embodiments, the gene associated with cancer is RAC1. In other embodiments, the mutant gene associated with cancer is RAC1 P29X, RAC1 P29S, RAC1 D65X or RAC1 D65N.

Inhibition of the gene, or mutant gene, associated with cancer, such as melanoma, can be assessed using a wide variety of methods, including those disclosed herein, as well as methods well-known in the art or to be developed in the future. That is, the routineer will appreciate, based upon the disclosure provided herein, that decreasing the level or activity of a gene, or mutant gene, associated with cancer, such as melanoma, can be readily assessed using methods that assess the level of a nucleic acid encoding the gene, or mutant gene, associated with cancer, such as melanoma, (e.g., mRNA) and/or the level of protein encoded by the gene, or mutant gene, associated with cancer, such as melanoma, present in a biological sample.

One skilled in the art, based upon the disclosure provided herein, would understand that the compositions and methods of the invention are useful in treating pathologies associated with cancer, such as melanoma, in subjects who have cancer, whether or not the subject is also being treated with other medication or chemotherapy. Further, the skilled artisan will further appreciate, based upon the teachings provided herein, that the pathologies associated with cancer treatable by the compositions and methods described herein encompass any pathology associated with cancer, such as melanoma, where RAC1, or mutant RAC1, plays a role.

An inhibitor composition of the invention can include, but should not be construed as being limited to, a chemical compound, a protein, a peptide, a peptidomemetic, an antibody, a ribozyme, a small molecule chemical compound, and an antisense nucleic acid molecule (e.g., siRNA, miRNA, etc.). One skilled in the art would readily appreciate, based on the disclosure provided herein, that an inhibitor composition encompasses a chemical compound that decreases the level or activity of a gene, or mutant gene, associated with melanoma. Additionally, an inhibitor composition encompasses a chemically modified compound, and derivatives, as is well known to one skilled in the chemical arts.

Further, one skilled in the art would, when equipped with this disclosure and the methods exemplified herein, appreciate that an inhibitor composition includes such inhibitors as discovered in the future, as can be identified by well-known criteria in the art of pharmacology, such as the physiological results of inhibition of a gene, or mutant gene, associated with melanoma, as described in detail herein and/or as known in the art. Therefore, the present invention is not limited in any way to any particular inhibitor as exemplified or disclosed herein; rather, the invention encompasses those inhibitors that would be understood by the routineer to be useful as are known in the art and as are discovered in the future.

Further methods of identifying and producing an inhibitor composition are well known to those of ordinary skill in the art, including, but not limited, obtaining an inhibitor from a naturally occurring source (i.e., Streptomyces sp., Pseudomonas sp., Stylotella aurantium). Alternatively, an inhibitor composition can be synthesized chemically. Further, the routineer would appreciate, based upon the teachings provided herein, that an inhibitor composition can be obtained from a recombinant organism. Compositions and methods for chemically synthesizing inhibitor composition and for obtaining them from natural sources are well known in the art and are described in the art.

One skilled in the art will appreciate that an inhibitor can be administered as a small molecule chemical, a protein, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a method of administering a protein or a nucleic acid encoding a protein that is an inhibitor of a gene, or mutant gene, associated with melanoma. (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

One skilled in the art will realize that diminishing the amount or activity of a gene, or mutant gene, that itself increases the amount or activity of a gene, or mutant gene, associated with cancer, such as melanoma, can serve in the compositions and methods of the present invention to decrease the amount or activity of the gene, or mutant gene, associated with cancer, such as melanoma. Therefore, inhibitor compositions that inhibit the amount or activity of a modulator of a gene, or mutant gene, associated with cancer, such as melanoma, are included in the compositions and methods of the invention. In some embodiments, the gene associated with cancer is RAC1. In other embodiments, the mutant gene associated with cancer is RAC1 P29X, RAC1 P29S, RAC1 D65X or RAC1 D65N. In various embodiments, the inhibited modulator of a gene, or mutant gene, associated with cancer, such as melanoma, is at least one of DOCK, mutant DOCK, VAV, mutant VAV, KARLN, mutant KARLN, TIAM1, mutant TIAM1, PREX2 and mutant PREX2.

One skilled in the art will also realize that increasing the amount or activity of a gene, or mutant gene, that itself decreases the amount or activity of a gene, or mutant gene, associated with cancer, such as melanoma, can serve in the compositions and methods of the present invention to decrease the amount or activity of the gene, or mutant gene, associated with cancer, such as melanoma. Therefore, activator compositions that activate the amount or activity of a modulator of a gene, or mutant gene, associated with melanoma, are included in the compositions and methods of the invention. In some embodiments, the gene associated with cancer is RAC1. In other embodiments, the mutant gene associated with cancer is RAC1 P29X, RAC1 P29S, RAC1 D65X or RAC1 D65N. In various embodiments, the activated modulator of a gene, or mutant gene, associated with cancer, such as melanoma, is at least one of GAP, mutant GAP, mutant DOCK, mutant VAV, mutant KARLN, mutant TIAM1, and mutant PREX2.

One skilled in the art will realize that diminishing the amount or activity of a gene, or mutant gene, that itself is an effector of a gene, or mutant gene, associated with cancer, such as melanoma, can serve in the compositions and methods of the present invention to decrease the amount or activity of the gene, or mutant gene, associated with cancer, such as melanoma. Therefore, inhibitor compositions that inhibit the amount or activity of an effector of a gene, or mutant gene, associated with cancer, such as melanoma, are included in the compositions and methods of the invention. In some embodiments, the gene associated with cancer is RAC1. In other embodiments, the mutant gene associated with cancer is RAC1 P29X, RAC1 P29S, RAC1 D65X or RAC1 D65N. In various embodiments, the inhibited effector of a gene, or mutant gene, associated with cancer, such as melanoma, is at least one of PAK1, mutant PAK1, PAK2, mutant PAK2, PAK3, mutant PAK3, PAK-4, mutant PAK-4, PAK5, mutant PAK5, PAK6, mutant PAK6, PAK7, mutant PAK7, JNK, mutant JNK, PLCβ, mutant PLCβ, p38, mutant p38, MEK, mutant MEK, ERK, and mutant ERK.

Antisense oligonucleotides are DNA or RNA molecules that are complementary to some portion of an mRNA molecule. When present in a cell, antisense oligonucleotides hybridize to an existing mRNA molecule and inhibit translation into a gene product Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The compositions and methods of the invention include the use of an antisense oligonucleotide to diminish the level of a gene, or mutant gene, associate with melanoma.

The compositions and methods of the invention also include the use of an antisense oligonucleotide to diminish the level of an activating modulator of the gene, or mutant gene, associated with melanoma, thereby causing a decrease in the amount or activity of the gene, or mutant gene, associated with melanoma.

Contemplated in the present invention are antisense oligonucleotides that are synthesized and provided to a cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.). Alternatively, inhibition of a gene, or mutant gene, associated with melanoma, or of a activating modulator of the gene, or mutant gene, associated with melanoma, can be accomplished through the use of a ribozyme. Using ribozymes for inhibiting gene expression is well known to those of skill in the art (see, e.g., Cech et al., 1992, J. Biol. Chem. 267:17479; Hampel et al., 1989, Biochemistry 28: 4929; Altman et al., U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of the molecule, one of ordinary skill in the art could synthesize an antisense oligonucleotide or ribozyme without undue experimentation, provided with the disclosure and references incorporated herein.

One skilled in the art will appreciate that inhibitors of a gene, or mutant gene, associated with melanoma can be administered singly or in any combination. Further, inhibitors can be administered singly or in any combination in a temporal sense, in that they may be administered simultaneously, before, and/or after each other. One of ordinary skill in the art will appreciate, based on the disclosure provided herein, that inhibitors can be used to treat pathologies associated with melanoma, and that an inhibitor can be used alone or in any combination with another inhibitor to effect a therapeutic result.

It will be appreciated by one skilled in the art, when armed with the present disclosure including the methods detailed herein, that the invention is not limited to treatment of a pathology associated with melanoma that is already established. Particularly, the pathology need not have manifested to the point of detriment to the subject; indeed, the pathology need not be detected in a subject before treatment is administered. That is, significant pathology associated with melanoma does not have to occur before the present invention may provide benefit. Therefore, the present invention includes a method for preventing a pathology associated with melanoma in a subject, in that an inhibitor, as discussed previously elsewhere herein, can be administered to a subject prior to the onset of a pathology, thereby preventing the pathology. The preventive methods described herein also include the treatment of a subject that is in remission for the prevention of a recurrence.

One skilled in the art, when armed with the disclosure herein, would appreciate that the prevention of a pathology associated melanoma encompasses administering to a subject an inhibitor as a preventative measure against a pathology associated with melanoma.

The invention encompasses administration of an inhibitor composition to practice the methods of the invention; the skilled artisan would understand, based on the disclosure provided herein, how to formulate and administer the appropriate inhibitor compositions to a subject. However, the present invention is not limited to any particular method of administration or treatment regimen.

### Pharmaceutical Compositions

Compositions identified as potential useful compounds for treatment and/or prevention of cancer, such as melanoma, can be formulated and administered to a subject for treatment of cancer, such as melanoma, as now described.

The invention encompasses the preparation and use of pharmaceutical compositions comprising a composition useful for treatment of cancer, such as melanoma, disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically-acceptable carrier” means a chemical composition with which an appropriate inhibitor thereof, may be combined and which, following the combination, can be used to administer the appropriate inhibitor thereof, to a subject.

The pharmaceutical compositions useful for practicing the invention may be administered to deliver a dose of between about 0.1 ng/kg/day and 100 mg/kg/day.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate inhibitor thereof, according to the methods of the invention.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. Preferably, the dosage of the compound will vary from about 1 mg to about 100 mg per kilogram of body weight of the animal. More preferably, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### A Recurring Activating Mutation in RAC1 in Melanomas

The MAP-kinase (MAPK/ERK) signaling pathway is frequently activated in melanomas, with recurrent activating mutations in BRAF and NRAS in about 50% and 12% of melanomas, respectively (Bauer et al., 2011 Pigment Cell Melanoma Res, 24:345; Bauer et al., 2006, 127:179). Exome sequencing of modest numbers of melanomas have been performed to search for new genes contributing to melanoma pathogenesis, and have suggested a role for a recurrent mutation in TRRAP (S722P) and diverse mutations in GRIN2A in cutaneous melanomas (Wei et al, 2011, Nat Genet, 43 442), leaving open the possibility that frequent recurrent oncogenic mutations remain to be discovered.

The materials and methods used in these experiments are now described.

### Melanoma Tumors and Cell Cultures

The melanoma tumors were obtained from different patients and were excised to alleviate tumor burden. Specimens were collected with participants' informed signed consent according to Health Insurance Portability and Accountability Act (HIPAA) regulations with a Human Investigative Committee protocol. The melanomas used for sequencing were from snap-frozen tumors or from short-term cultures (Halaban et al., 2010, Pigment Cell Melanoma Res, 23:190). Most of the melanoma cells were collected after 0-4 passages in culture, except for one sample entitled YURIF that was collected after 14 passages in culture.

### DNA Extraction

The DNeasy purification kit (Qiagen Inc., Valencia, Calif.) was used to extract the DNA from cell pellets and freshly frozen tumors. The OneStep™ PCR Inhibitor Removal Kit (Zymo Research Corporation, Irvine, Calif.) was used for samples with high melanin content.

### Exome Capture, High Throughput Sequencing and Sequence Validation

Genomic DNA was captured to the SeqCap EZ Exome Library v1.0 by solution-based capture method following the manufacturer's protocols (Roche/NimbleGen) at the Yale Center for Genome Analysis. DNA was sheared by sonication and adaptors were ligated to the resulting fragments. The adaptor-ligated templates were fractionated by agarose gel electrophoresis and fragments of the desired size were excised. Extracted DNA was amplified by ligation mediated PCR, purified, and hybridized to the SeqCap EZ Exome Library v1.0 at 42.0° C. using the manufacturer's buffer. Hybridized DNA was pulled down with Streptavidin beads and amplified by ligation-mediated PCR. The resulting fragments were purified and subjected to DNA sequencing on the Illumina platform. Captured and non-captured amplified samples were subjected to quantitative PCR to measure the relative fold enrichment of the targeted sequence. Captured libraries were sequenced on the Illumina Genome Analyzer (GA) IIx as 75-bp paired-end reads, following the manufacturer's protocols. Image analysis and base calling was performed by Illumina pipeline version 1.6 and 1.7 with default parameters.

Illumina GAIIx sequencing data was validated by Sanger sequencing of 315 gene specific amplicons.

Furthermore, the frequency of the recurrent RAC1-P29S mutation identified in the original 57 melanoma samples was assessed in 289 additional melanoma, 11 nevi specimens and matching germline DNA available at the Tissue Resource Core of the Yale SPORE in Skin Cancer, employing Sanger sequencing and the TaqMan® assay (Applied Biosystems, Carlsbad, Calif.). The Sanger sequencing primers used to assess RAC1-P29S mutation in the expanded cohort were F1: 5′-ACCTAAACAGAATGTGATGGCTCC-3′ (SEQ ID NO:1) and B1: 5′-GGTCAAAGAAATGTGAAACCCG-3′ (SEQ ID NO: 2), optimal annealing temperature of 51.7° C., generating a 374 bp product.

The RAC1 D65N mutation was validated in one acral melanoma using the following primers RAC1[F2] 5′-AAGTTTTGCCCGTGCCGCCTTCCTC-3 (SEQ ID NO: 3) and RAC1[B2] 5′-CCAATCCCACCCTGTTTGCTATTTAC-3′ (SEQ ID NO: 4), optimal annealing temp 56.9° C., generating a 423 bp product.

In addition, another cohort of 94 melanoma cell lines in the Oncogenomics Laboratory, Queensland Institute of Medical Research was tested by Sanger sequencing for the RAC1 P29S mutation using BigDye Terminator v3.1 chemistry on a 3730×1 DNA Analyzer (Applied Biosystems). Primers used were F: 5′-TGGTGATAAAGGGTTATAGAAAACA-3′(SEQ ID NO: 5) and R: 5′-CAGCAAAACAAATGGTCAAA-3′ (SEQ ID NO: 6) to generate a 299 bp product using an annealing temperature of 58° C.

### RAC1 Amino Acid Sequence

Rac1 protein (GenBank: CAB53579.5; NP 008839) amino acid sequence:

(SEQ ID NO: 7)
MQAIKCVVVGDGAVGKTCLLISYTTNAFPGEYIPTVFDNYSANVMVDG
KPVNLGLWDTAGQEDYDRLRPLSYPQTVGETYGKDITSRGKDKPIADV
FLICFSLVSPASFENVRAKWYPEVRHHCPNTPIILVGTKLDLRDDKDT
IEKLKEKKLTPITYPQGLAMAKEIGAVKYLECSALTQRGLKTVFDEAI
RAVLCPPPVKKRKRKCLLL

### RAC1 P29X Amino Acid Sequence

Rac1 P29X amino acid sequence, where X is any amino acid:

(SEQ ID NO: 8)
MQAIKCVVVGDGAVGKTCLLISYTTNAFXGEYIPTVFDNYSANVMVDG
KPVNLGLWDTAGQEDYDRLRPLSYPQTVGETYGKDITSRGKDKPIADV
FLICFSLVSPASFENVRAKWYPEVRHHCPNTPIILVGTKLDLRDDKDT
IEKLKEKKLTPITYPQGLAMAKEIGAVKYLECSALTQRGLKTVFDEAI
RAVLCPPPVKKRKRKCLLL

### RAC1 P29S Amino Acid Sequence

Rac1 P29S amino acid sequence:

(SEQ ID NO: 9)
MQAIKCVVVGDGAVGKTCLLISYTTNAFSGEYIPTVFDNYSANVMVDG
KPVNLGLWDTAGQEDYDRLRPLSYPQTVGETYGKDITSRGKDKPIADV
FLICFSLVSPASFENVRAKWYPEVRHHCPNTPIILVGTKLDLRDDKDT
IEKLKEKKLTPITYPQGLAMAKEIGAVKYLECSALTQRGLKTVFDEAI
RAVLCPPPVKKRKRKCLLL

### RAC1 D65X Amino Acid Sequence

Rac1 D65X amino acid sequence, where X is any amino acid:

(SEQ ID NO: 10)
MQAIKCVVVGDGAVGKTCLLISYTTNAFPGEYIPTVFDNYSANVMVDG
KPVNLGLWDTAGQEDYXRLRPLSYPQTVGETYGKDITSRGKDKPIADV
FLICFSLVSPASFENVRAKWYPEVRHHCPNTPIILVGTKLDLRDDKDT
IEKLKEKKLTPITYPQGLAMAKEIGAVKYLECSALTQRGLKTVFDEAI
RAVLCPPPVKKRKRKCLLL

### RAC1 D65N Amino Acid Sequence

Rac1 D65N amino acid sequence:

(SEQ ID NO: 11)
MQAIKCVVVGDGAVGKTCLLISYTTNAFPGEYIPTVFDNYSANVMVDG
KPVNLGLWDTAGQEDYNRLRPLSYPQTVGETYGKDITSRGKDKPIADV
FLICFSLVSPASFENVRAKWYPEVRHHCPNTPIILVGTKLDLRDDKDT
IEKLKEKKLTPITYPQGLAMAKEIGAVKYLECSALTQRGLKTVFDEAI
RAVLCPPPVKKRKRKCLLL

### Single Nucleotide Variant

The following procedure was followed for discovery of recurrent somatic variants in melanoma. Illumina sequencing of paired tumor and germline DNA was used for the automated calling of somatic single nucleotide variants (SNVs) in the discovery set (27 matched tumor/germline pairs), and then additional Illumina sequencing of 30 unmatched melanomas in a validation set was done to identify recurrent somatic variants identified in the discovery set. For both sets, novel SNVs that occurred in the tumors were identified. For the discovery set, sequencing of germline DNA was used to distinguish between somatic and inherited variants. For the validation set, germline DNA was selectively sequenced by Sanger sequencing to determine whether a variant is somatic.

### Reference Genome and RefSeq Database

Human reference genome GRCh37/hg19 was used for mapping Exome-Seq data. The RefSeq sequence database downloaded from NCBI on 2011-5-12 was used as the gene model and for determining amino acid substitutions.

### Exome-Seq Processing

The sequence fidelity and read coverage of all Illumina Genome Analyzer IIX runs was first tested. In general, there was an excellent low average sequencing error rate of 0.23%, representing the fraction of bases from sequencing reads that do not align with the reference genome. The average coverage was 65±14.8 independent reads per targeted base pair (minimum mean sample coverage 30, maximum mean sample coverage 93). The following procedure was used to call melanoma sequence variations: Reads were first trimmed based on their quality scores using the program BTrim (Kong et al., 2011, Genomics, May 30). The reads were then mapped against the reference genome using Burrows-Wheeler Aligner (BWA) (Li et al., 2009, Bioinformatics, 25:1754). SAMtools version 0.1.8-11 (r672) (Li et al., 2009, Bioinformatics, 25:2078) was used for PCR duplicate removal and SNV calling. Annotations of SNVs were performed using MU2A (Garla et al., 2011, Bioinformatics, 27:416). Annotation files were checked for adjacent pairs of SNVs affecting the same codon. If present, sequencing reads were scanned for occurrence of both SNVs on a single allele, and the amino acid change was predicted based on the simultaneous mutations. SNVs were filtered according to the quality criteria discussed herein, and were considered variants when not included in repositories of common variations, including dbSNP132 and the latest 1,000 genome data.

For the discovery screen, novel SNVs were classified into somatic and inherited variations as follows: the tumor SNV was first called, and then sequencing data in a matched germline DNA sample was used to determine the presence or absence of variant reads at the same position. The SNV was called somatic in the absence of variant reads in the germline DNA samples.

### Somatic SNV Call Precision Based on Sanger Validations

The precision analysis for the two-step somatic calling pipeline was as follows: the precision of calling a tumor SNV was first established (establishing the presence of the variant in tumor), and then the precision of classifying it as a somatic variant was determined based on matched germline DNA data. Precision was defined as the ratio of correctly called variants over all called variants.

The precision of calling tumor SNVs was established by comparing 315 Exome-Seq tumor SNV calls across 57 melanoma samples to results from Sanger validation experiments of the same SNVs. Sanger sequencing validated 258 of these SVNs, whereas 57 were found to be false positives. The goal was to determine sequencing statistics at which most of the true positive cases are called, and the false positives are rejected. Two statistics were evaluated for calling variants. One was the Mutant Allele Frequency (MAF), i.e., the number of variant reads over all reads at a particular position, as determined by Illumina GAIIx sequencing. Variant allele frequencies that fell below 15 percent failed to validate in Sanger sequencing. Accordingly, a variant allele frequency threshold of 15% or more was set when calling variants. The second method used was the Samtools SNP score, which represents the negative log of the probability of erroneously calling a SNV. A conservative SNP score >100 was chosen for calling SNVs. The final thresholds were: 1) SNP score >100; 2) MAF≧15%, and two further thresholds; 3) at least one forward and one backward read; and 4) minimum coverage of 8 reads at the variant position. At these thresholds, 255 of the 315 SNVs were retained, 16 of which were false positives. A precision of 239/255=93.7% was thus calculated for calling tumor variants in Exome-Seq. In the presence of a matched germline DNA sample, a tumor variant was called as either somatic or inherited. To determine somatic call precision, Sanger sequencing was used to determine how many of the somatic calls were actually inherited SNVs, or false positive tumor SNVs that are erroneously called somatic. Ninety-four tumor SNVs in the Sanger validation set had matched germline DNA sequencing data: 70 of those were true somatic variants, 18 were inherited, and 6 were false positive tumor SNVs. The sequencing pipeline automatically called 76 out of the 94 SNVs as somatic. Of those, 69 were true positive somatic SNVs, one was an inherited SNV, and 6 were false positive tumor SNVs. A somatic call precision of 69/(69+1+6)=90.7% was thus calculated.

### Somatic SNV Call Sensitivity Based on Detection of SNVs in Germline DNA

The true total number of somatic changes in tumor is not known yet, and there is a need to assess somatic call sensitivity, which is defined as the number of called variants over all real variants. A somatic call sensitivity estimate was designed based on the two-step somatic call procedure: First, the sensitivity of detecting the presence of a variant in tumor was determined. Then, the sensitivity of detecting those variants that are somatic was established using matched germline DNA sequencing data. For the estimation, it was assumed that detection of SNVs in tumor can be equated to detecting inherited tumor SNVs given adequate tumor purity. Therefore, the sensitivity of detecting tumor SNVs was estimated by counting the number of known SNPs in germline DNA, which are positively called in a matched tumor for all 27 tumor/normal pairs, measuring a mean sensitivity of 90.6%. The ability to detect those tumor SNVs that are somatic was then measured. Using the Sanger-validated 70 somatic tumor SNVs with corresponding sequencing data in germline DNA, 69 of those variants were correctly detected at the discussed thresholds, a somatic call sensitivity of 69/70=98.6%. Overall sensitivity to detect somatic variants is thus estimated to be 0.906×0.986=89.3%.

### Somatic SNV Call Sensitivity for Known Melanoma Driver Mutations

Routine Sanger sequencing of all 57 melanomas in the discovery and validation screens determined 18 BRAF-V600 (V600E/K/R) and 11 NRAS (Q61L/R/H) mutations. The sensitivity of Exome-Seq to identify these variants was determined At the thresholds discussed earlier, Exome-Seq automatically called all but one BRAF, and all NRAS variants, for a SNV detection sensitivity of 28/29=96.5%. Likewise, of the 14 BRAF and NRAS variants with matched germline DNA sequencing data, Exome-Seq automatically called all but one BRAF, and called all NRAS mutations, as somatic, for a somatic call sensitivity of 13/14=92.8%. The failed BRAF call in tumor was due to high level of fibroblast contamination in the cell culture (80%). BRAF and NRAS mutations likely occur early in melanoma genesis, and are thought to be present across all tumor clones. Difficulties in detecting these mutations are therefore primarily caused by stromal tissue contamination, as opposed to clonal heterogeneity. The fact that most of these variants were recovered indicates that the sequencing depth and sensitivity are sufficient to retrieve variants with similar clonal distributions as BRAF and NRAS mutations.

DNA Analysis from Healthy Population

A TaqMan® assay to detect the RAC1-P29S mutation was designed using the Applied Biosystem software on their web site. The assay was validated on DNA samples that sequencing had shown to have the mutation; it was then used to test germline DNA samples of patients and additional tumors not sequenced. In addition, 2,596 samples of individuals from 57 anthropologically defined populations originating from diverse parts of the world (Donnelly et al., 2010, Am J Hum Genet, 86: 161) were genotyped. Analyses were done in 384-well plates using the manufacturer's protocol except that volume was reduced to 3 μl. After 30 cycles, the plates were read on an AB 9600HD using SDS software.

Site Directed Mutagenesis and Transient Ectopic Rac1 expression

The pBabe-CyPet-Rac1 Retroviral expression vector and pcDNA3-EGFP-RAC1 plasmid were purchased from Addgene Inc. (Cambridge, Mass.). The plasmids were constructed by as previously described (Wang et al., 2010, Nat Cell Biol, 12:591; Wu et al., 2009, Nature, 461:104; Machacek et al., 2009, Nature, 461:99). The retroviral expression construct contains a protease site that allows RAC1 to be cleaved from the CyPet tag in the mouse melanocytes. The P29S mutation was introduced in each of the plasmids with the QuikChange kit (Stratagene, La Jolla, Calif.) employing the following primers: F: 5′ GTTACACAACCAATGCATTTTCTGGAGAATATATCCCTACTG 3′ (SEQ ID NO:12) and R: 5′CAGTAGGGATATATTCTCCAGAAAATGCATTGGTTGTGTAAC 3′ (SEQ ID NO: 13).

The mutation in the vector was validated by sequencing the plasmids with the primer B: 5′-TTTGCGGATAGGATAGGGGGCG-3′ (SEQ ID NO:14), and the mutation within the infected RAC1 cells was validated with F: 5′-TCAAGTGTGTGGTGGTGGGAG-3′ (SEQ ID NO:15) B: 5′-TTTGCGGATAGGATAGGGGGCG-3′ (SEQ ID NO:16).

Mouse melanocytes (from C57BL mice) at passage 19 were infected with retroviruses encoding pBabe-CyPet-RAC1 wild type or RAC1-P29S. Puromycin (1 μg/ml) was added two days later, and the melanocytes were tested for cell proliferation and migration after 10 days selection with the drug.

### Fluorescence Microscopy

COS-7 cells were transiently transfected at 90% confluence in 10 cm plates with 1.5 μg pcDNA3-EGFP-RAC1-WT and RAC1-P29S constructs using Lipofectamine 2000 (Invitrogen) according to manufacturer's instructions. Following transfection, cells were detached and plated in 24-well trays on Fisherbrand no. 1.5 coverslips (Cat #12-545-81) and cultured in Dulbecco's Modified Eagle Medium (DMEM) supplemented with 10% fetal bovine serum (PBS) and 1% penicillin/streptomycin for 24 hours to recover from transfection. Cells on coverslips were washed 3 with DMEM with 1% PBS then were fixed by incubating cells for 15 min at room temperature in 1 ml paraformaldehyde (3.2%). Cells were then washed 3× with DMEM with 1% PBS and coverslips were mounted onto microscope slides using ProLong Gold antifade mountant (Invitrogen, CAT #P36935). Cells were examined with a multicolor spinning-disk confocal UltraVIEW VoX system (Perkin-Elmer) based on an inverted Olympus microscope (1×−71) equipped with a 1 Kb×1 Kb EM charge-coupled device camera (Hamamatsu Photonics) using a 60×1.4 NA oil objective lens. The system was controlled by Velocity software.

### Western Blot Analyses

Total cell extracts (16 μg protein/lane) with concentrations estimated with the BioRad kit (Bio-Rad Laboratories, Hercules, Calif.), were subjected to Western blot analysis (Halaban et al., 2009, PLoS ONE, 4:e4563). The membranes were probed with the anti-RAC1 mouse monoclonal against recombinant full-length protein (clone 23A8, Millipore cat#05-389), anti-phospho-Erk2 pThr202/Tyr204 (mAb), ERK1/2 (ERK 1/2, 137F5) (from Cell Signaling Technology, Beverly, Mass.), and anti-β-Actin mAb (A1978 from Sigma-Aldrich, St. Louis, Mo. 63103).

### Cell Proliferation and Migration Assays

Cell proliferation assays were performed in 6-well plates (2,000 cells/well) in triplicate wells in the presence of puromycin (1 μg/ml). The mouse melanocytes were incubated in OptiMEM supplemented with antibiotics and 7% horse serum in the presence and absence of the required growth factor TPA (10 ng/ml, 12-O-tetradecanoylphorbol-13-acetate, Sigma). The cells were harvested at 2-day intervals and counted with a Coulter counter.

Cell migration was measured with the Cultrex 24 Well Cell Migration Assay (Trevigen, Inc. Gaithersburg, Md., cat number 3465-024-K) following the manufacturer's instructions.

### RAC1-P29S and RAC1-WT Expression and Purification

RAC1-P29S spanning residues 2-177 was sub-cloned into a modified pET-28 vector with a six-histidine N-terminal tag followed by GST and cleavable by thrombin and TEV proteases. Recombinant RAC1-P29S was expressed as an N-terminal fusion with glutathione-S-transferase (GST) in BL21(DE3) cells, and induced with 1 mM IPTG for 12 hours at 30° C. Briefly, the fusion protein was affinity purified and cleaved by thrombin at 4° C. overnight. The protein was then loaded on a Superdex 200 HiLoad 16/60 (GE Healthcare) column in a buffer of 20 mM Tris-HCl (pH 8.0), 150 mM NaCl, and 1 mM (DTT). Purified mutant protein was finally concentrated to 3.5 mg/ml in a buffer of 20 mM Tris, 150 mM NaCl, 1 mM DTT, 5 mM MgCl₂, with and without 1 mM GMP-PNP.

For RAC1-WT residues 2-177 was sub-cloned into a modified pET-28 vector with a six-histidine N-terminal tag. RAC1-WT was expressed as an N-terminal fusion in BL21(DE3) cells, and induced with 1 mM IPTG for 12 hours at 30° C. Briefly, RAC1-WT was affinity purified and loaded on a Superdex 75 (GE Healthcare) column in a buffer of 20 mM Tris-HCl (pH 8.0), 150 mM NaCl, and 1 mM DTT. Purified RAC1-WT was finally concentrated to 7 mg/ml in a buffer of 20 mM Tris, 150 mM NaCl, 1 mM DTT, 5 mM MgCl₂, and 1 mM GMP-PNP.

### RAC1-P29S and RAC1-WT Crystallization

Crystals of RAC1-P29S were grown by vapor diffusion hanging drops formed by mixing a 1:1 volume ratio of purified RAC1-P29S and reservoir solution containing 0.1M HEPES (pH 7.5), 20% (v/v) PEG 4,000, and 7% (v/v) isopropanol at room temperature. RAC1-P29S crystals belong to space group P2₁2₁2₁ with unit cell dimensions a=50.3 Å, b=80.0 Å, c=94.9 Å, and α, β, γ=90°. There were two molecules per asymmetric unit. Crystals were equilibrated in a cryoprotectant buffer containing reservoir buffer plus 30% (v/v) ethylene glycol and were flash frozen in a nitrogen stream at 100K. X-ray data from a single crystal were collected to 2.1 Å resolution at the Yale Chemical and Biophysical Instrumentation Center using a Rigaku HF007 generator and a Saturn 944+ CCD detector.

A second crystal form was determined to 2.6 Å resolution from identical crystallization conditions in the space group P22₁2₁ with unit cell dimensions a=40.6 Å, b=51.9 Å, c=99.3 Å, and α, β, γ=90°. This crystal form has similar packing to the P212121 crystal, and is conformationally similar (RMSDs of 0.5 Å and 0.3 Å over 177 and 176 Ca atoms when compared to chains A and B respectively) so the analyses were conducted using the P2₁2₁2₁ crystal.

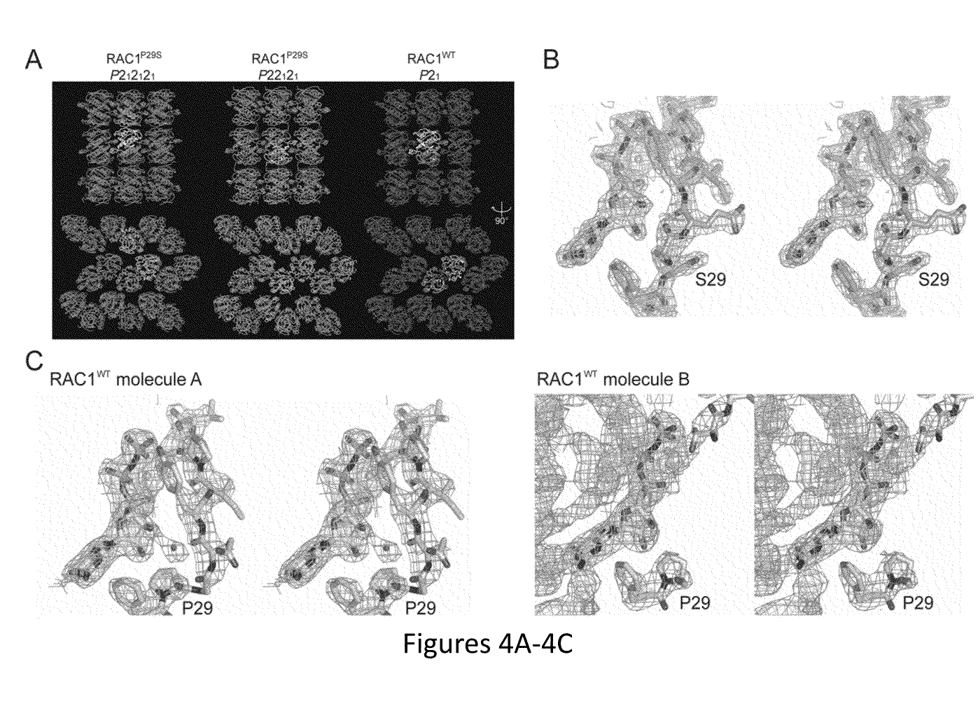

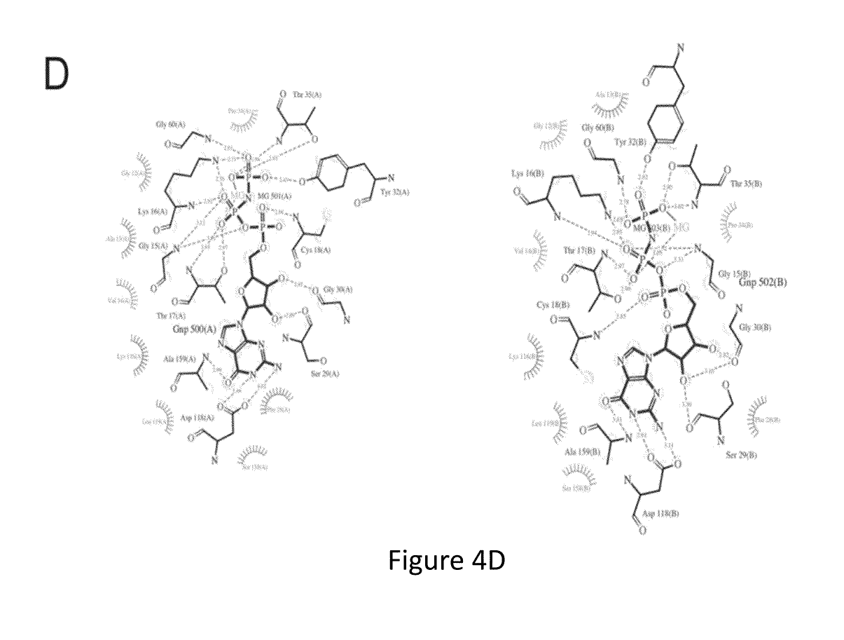

Crystals of RAC1-WT were grown in almost identical conditions in the space group P2₁ with unit cell dimensions a=40.9 Å, b=97.9 Å, c=51.7 Å, and β=96.6°. This crystal form has similar packing to both of the RAC1-P29S crystals allowing us to investigate whether the conformational changes observed for RAC1-P29S were due to crystal packing (FIG. 4).

### Structure Determination and Refinement

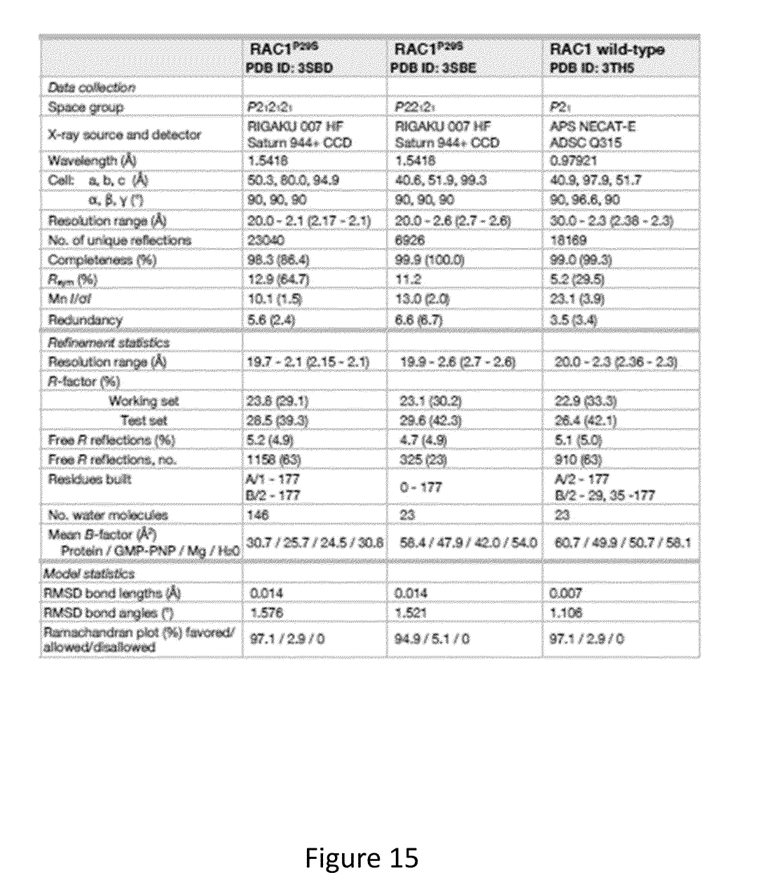

For the RAC1-P29S P2₁2₁2₁ crystal form, data were processed using the HKL2000 package (Otwinowski and Minor, eds., 1997, Processing of X-ray diffraction data collected in oscillation mode, 276:307-326) and initial phases calculated by molecular replacement using the program Phaser (McCoy et al., 2005, Acta Crystallogr D Biol Crystallogr, 61:458; Hirshberg et al., 1997, Nat Struct Biol, 4:147). Wild-type RAC1 (PDB ID: 1 MH1) (Hirshberg et al., 1997, Nat Struct Biol, 4:147) was used as a search model and yielded translation Z-scores of 19.2 and 47.1 for the two molecules in the asymmetric unit. Automated model building with ARP/wARP (Perrakis et al., 2001, Acta Crystallogr D Biol Crystallogr, 57:1445) built residues 2-176 in molecule A and residues 3-175 in molecule B, thus avoiding potential problems with model bias. Cycles of refinement and manual model building were conducted using REFMACS (Murshudov et al., 1997, Acta Crystallogr D Biol Crystallogr, 53:240) with a maximum-likelihood target, two TLS groups per molecule and medium NCS and using COOT (Emsley et al., 2004, Acta Crystallogr D Biol Crystallogr, 60:2126). Model validation was conducted using MolProbity (Chen et al., 2010, Acta Crystallogr D Biol Crystallogr, 66:12). The final refined model of RAC1-P29S has R and Rfree values of 24.0% and 28.5% (FIG. 15). 100% of residues fall within favored or allowed regions of the Ramachandran plot. Good electron density is observed throughout the structure, including GMP-PNP and the switch I region (FIG. 4). The structure is deposited in the PDB under accession code 3SBD.

A similar processing, solution and refinement protocol was conducted for the 2.6 Å P22₁2₁ structure of RAC1-P29S (FIG. 15), and the data have been deposited in the PDB under accession code 3SBE. Good electron density is observed throughout this structure, including GMP-PNP and the switch I region. A similar processing, solution and refinement protocol was conducted for the 2.3 Å P21 structure of RAC1-WT (FIG. 15), and the data have been deposited in the PDB under accession code 3TH5. Good electron density is observed throughout this structure, including GMP-PNP, however, the switch I regions of both molecules in the asymmetric unit are not well defined (FIG. 4). For molecule A the switch I loop displays poor electron density and for molecule B the switch I loop is not visible in the electron density. The crystal structure of RAC1-WT has similar lattice interfaces to RAC1-P29S illustrating that the conformational differences observed in switch I are not due to crystal packing effects.

Overall, the two RAC1-WT molecules are globally similar to the RAC1-P29S structures (molecule A has RMSDs of 0.44 Å, 0.31 Å and 0.34 Å over 173, 173 and 174 residues when compared to the P2₁2₁2 A, B and P22₁2₁ crystals, molecule B has RMSDs of 0.39 Å, 0.35 Å and 0.43 Å over 170, 169 and 171 residues when compared to the P2₁2₁2₁ A, B and P22₁2₁ crystals).

### RAC1 Activity Assay

A PAK1 pull-down assay (Millipore) was used to assess RAC1-WT and RAC1-P29S activity. Recombinant N-terminal His-tagged RAC1-WT and RAC1-P29S were purified by affinity and size-exclusion. The proteins were dialyzed for 12 hours against buffer containing 20 mM Tris-HCl (pH 8.0), 0.15M NaCl, 1 mM DTT, and 10 mM EDTA, followed by 2× dialysis for 12 hours against the same buffer without EDTA to discharge innately bound nucleotides. His-RAC1-WT or His-RAC1-P29S (6 μg) were incubated with 1 mM of nucleotide and GST-PAK1-PBD (GST fusion-protein corresponding to the p21-binding domain, PBD, residues 67-150 of human PAK-1, bound to glutathione agarose) (5 μg), for 3 hours, at 4° C. in a buffer containing 20 mM Tris-HCl (pH 8.0), 0.15M NaCl, 1 mM DTT and 10 mM MgCl₂. The beads were sedimented by centrifugation, the pellets washed 3× with the same buffer, the bound proteins were eluted with SDS-sample buffer at 95° C. and analyzed by Western blot with anti-polyHistidine antibody (clone HIS-1, H1029, Sigma-Aldrich). Incubations included no addition, GDP, GTP, GMP-PNP or GTPγS, NC (negative control) indicates GST-PAK1-PBD without any loaded RAC1 protein. One mg of His-RAC1-WT and His-RAC1-P29S were included as controls.

The results of the experiments are now described.

### Melanoma Mutations

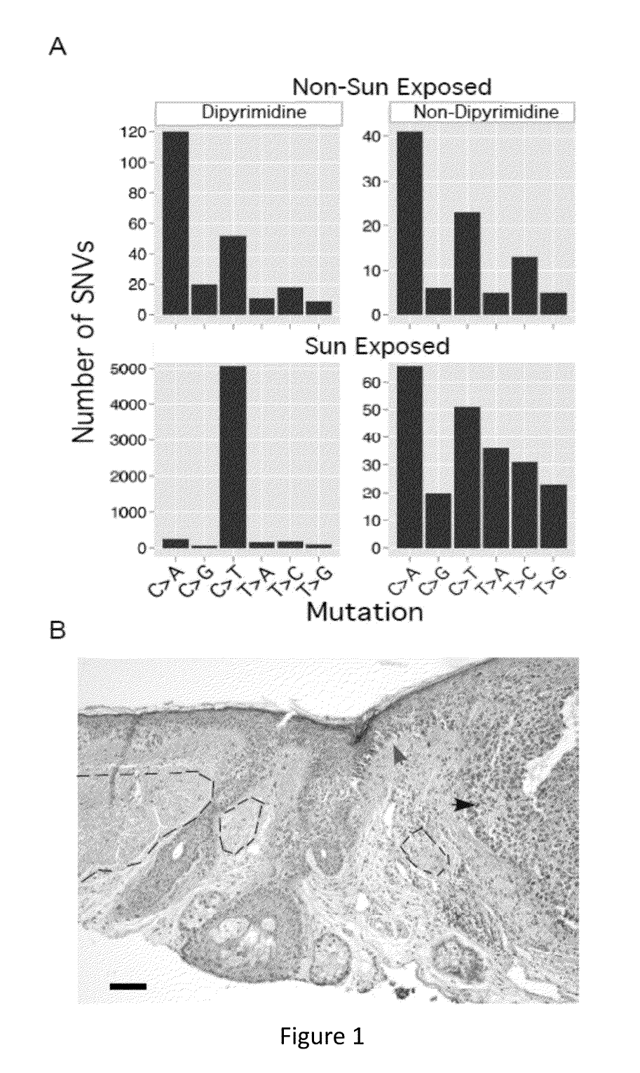

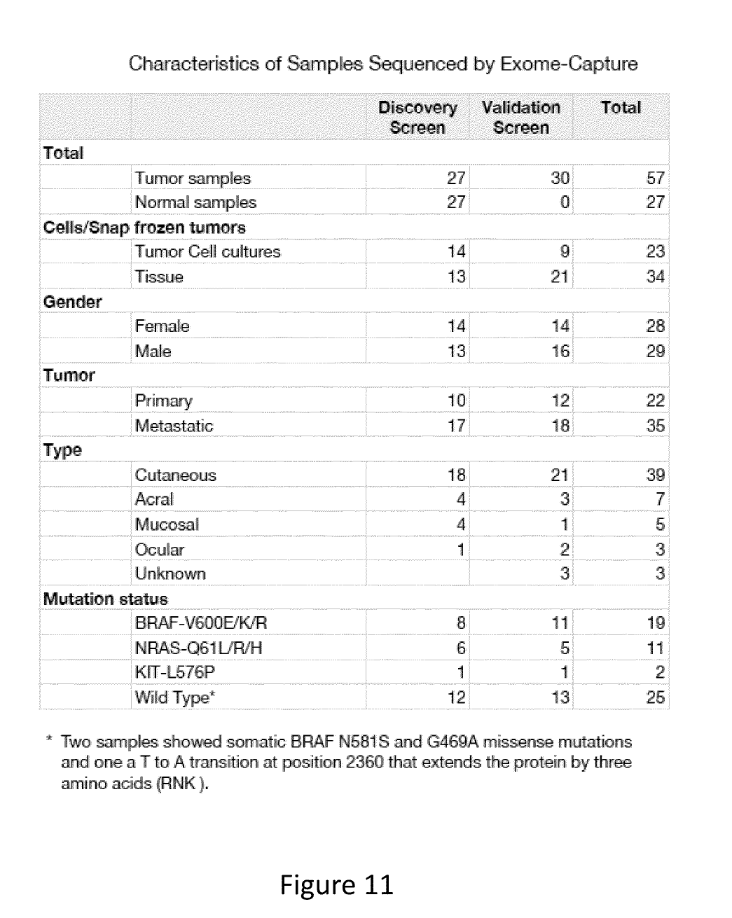

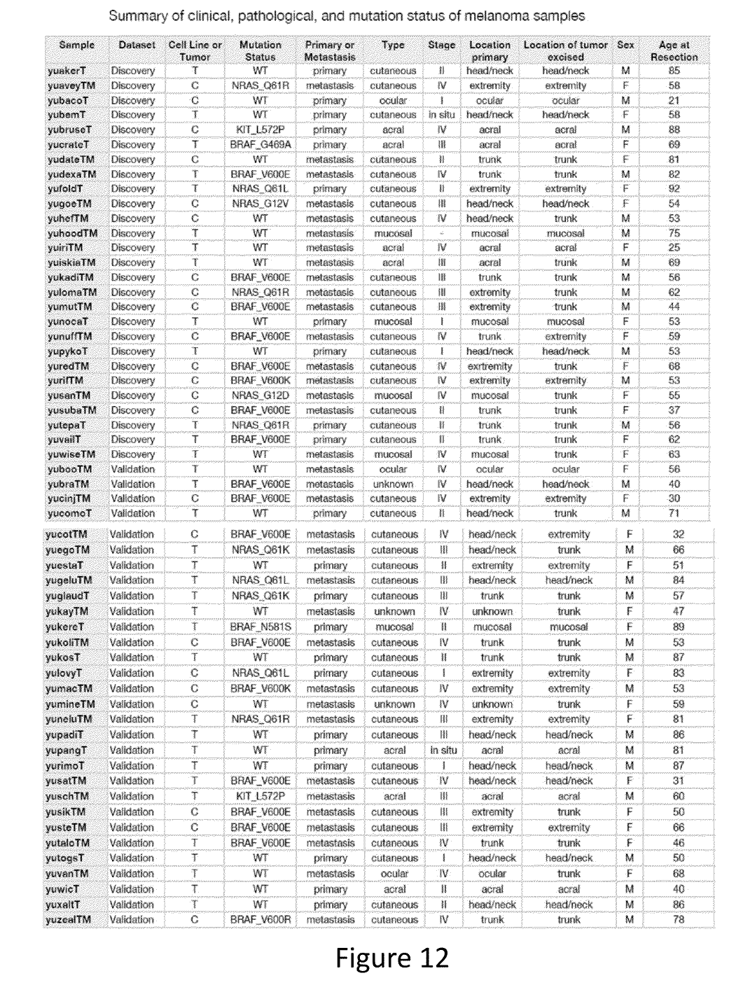

Exome sequencing (Exome-Seq) of 57 melanomas, comprising primary and metastatic cutaneous, acral, mucosal and ocular melanomas, was performed (FIGS. 11 and 12). This included sequencing of 27 melanomas with matched normal DNA (Discovery screen, FIG. 12) and 30 without (Validation screen, FIG. 12). The discovery screen identified 6,034 somatic non-silent SNVs (5,637 missense, 396 premature termination, and 1 frame shift), while the validation screen detected 7,976 novel non-silent SNVs. The mutation spectrum of the somatic coding SNVs was analyzed in sun-exposed (cutaneous) and non-sun exposed (acral, mucosal and ocular) melanomas separately. Analysis of the somatic coding mutations showed that sun-exposed melanomas exhibited a dramatic increase in the average number of mutations (mean 342 vs. 28 in non-sun-exposed melanomas), accounted for by an excess of C→T mutations in dipyrimidine sequences, a hallmark of UV signature mutations (FIG. 1A). This observed increase corresponded to frequent solar elastosis in sun damaged skin adjacent to cutaneous melanomas (FIG. 1B).

The discovery set identified 96.5% of the expected of BRAF (V600E/K/R) and NRAS (Q61L/R/H) mutations and all KIT (L576P) and CTNNB1 (S33C) mutations. Novel SNVs from the discovery and validation screens were then compared to somatic SNVs from prior melanoma sequencing efforts. The largest overlap was seen with the recent screen by Wei and colleagues (Wei et al, 2011, Nat Genet, 43:442), with 37 shared

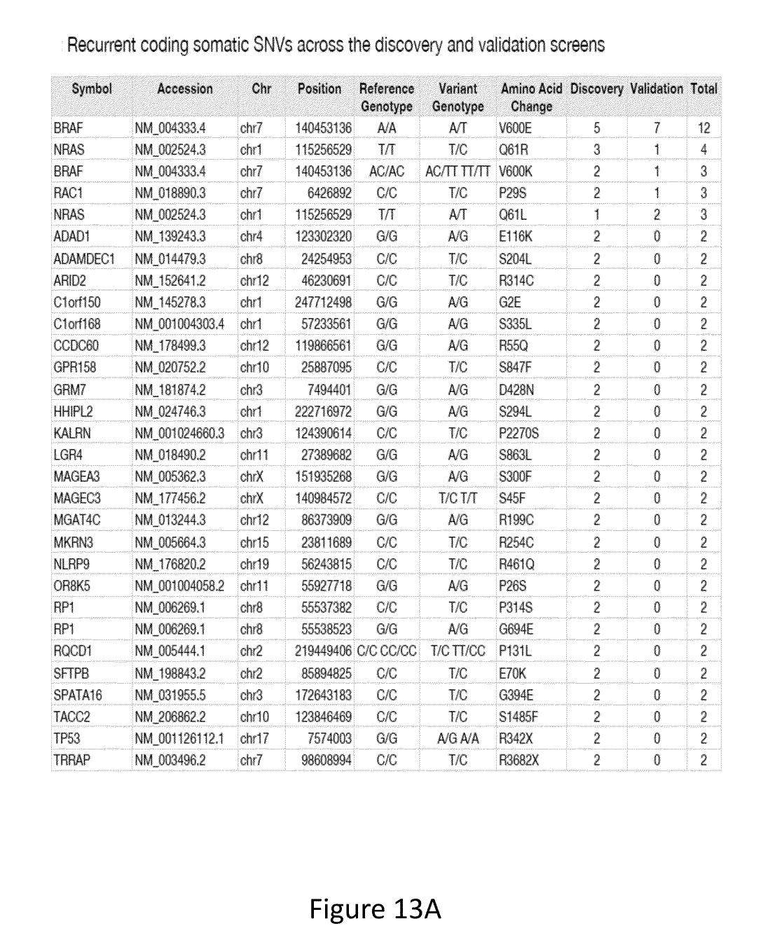

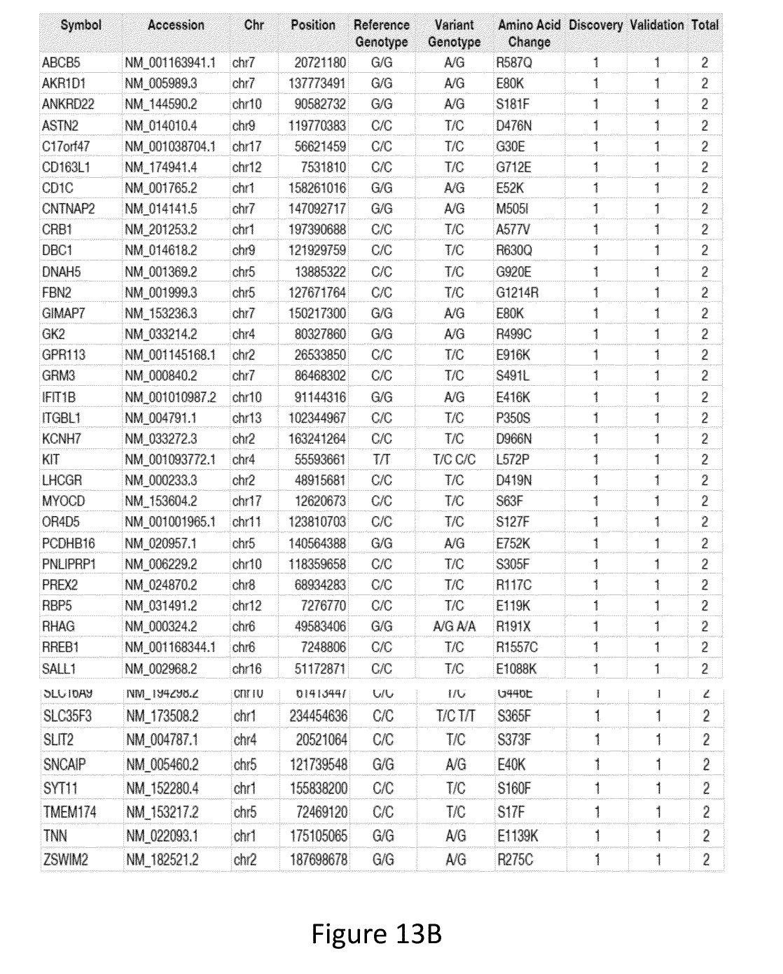

SNVs in coding regions. For example, the single somatic mutation reported in DBC1 (R630Q), occurred twice in the 57 exomes, and the DCC (G55E) mutation, previously seen three times, occurred once in the tumors examined GRIN2A (glutamate receptor, ionotropic, N-methyl D-aspartate 2A) was reported to harbor 34 distinct mutations in 135 samples (25.2%) (Wei et al, 2011, Nat Genet, 43:442). Five different somatic mutations in this gene were found, four mapping to the NMDAR2_C domain. Similarly, different loss of function somatic mutation, and one inherited mutation in BAP1 (BRCA1 associated protein-1), have recently been reported in uveal melanoma, a large fraction of them leading to early protein termination (Koga et al., 2009, Genome Res, 19:1462). Interestingly, indels in two ocular melanomas were identified, which were validated by Sanger sequencing. In one metastatic tumor (YUVAN), there was a somatic two-base homozygous deletion at codon 152 that leads to early termination. The other was identified in primary ocular melanoma cells (YUBACO, first passage in culture) from a 21-year-old patient as a frameshift mutation resulting from a two-base deletion at codon 153 leading to premature protein termination at codon 158. This indel was also present in the germline DNA of the melanoma patient and his unaffected mother; the maternal great grandfather also had ocular melanoma, strongly supporting this mutation being pathogenic. In addition to the known melanoma mutations (BRAF, NRAS and KIT), one novel recurrent mutation was identified in three melanomas, and 62 were found in two melanoma samples, including an early protein termination mutation in TRRAP (R3682X) in two independent melanomas (FIG. 13), which together lend further support to a role for TRRAP in cutaneous melanoma (Wei et al, 2011, Nat Genet, 43:442).

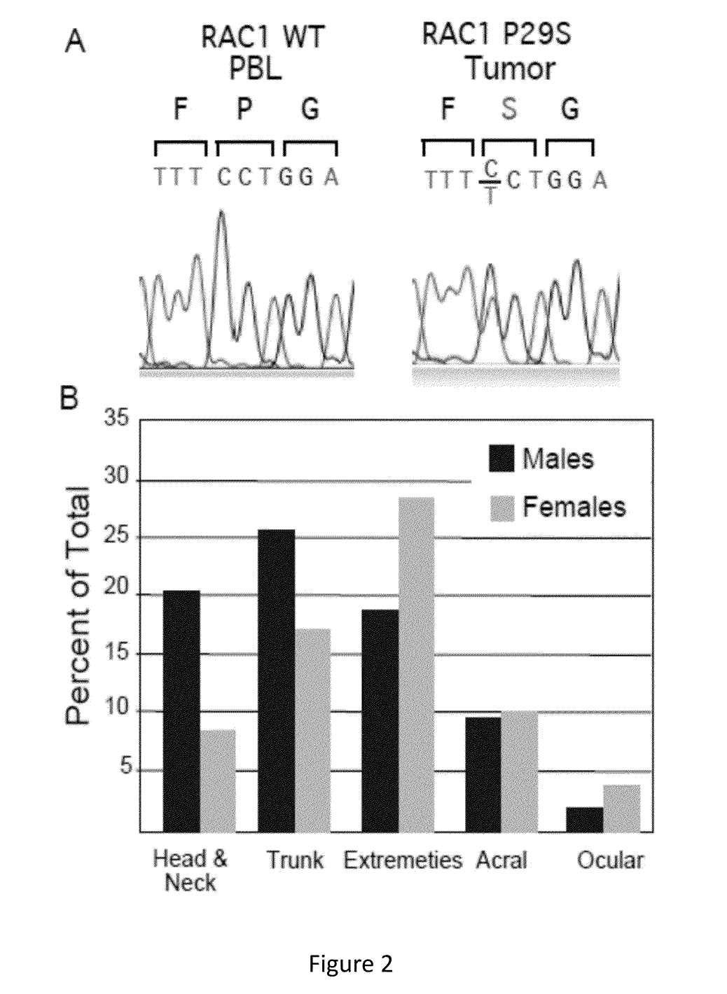

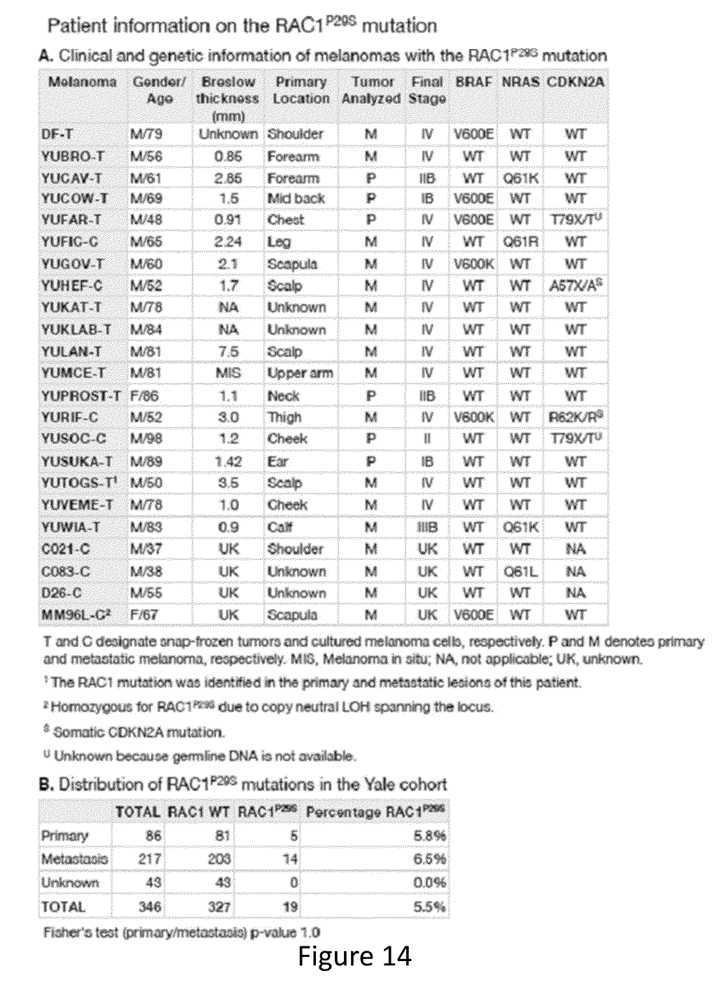

The mutation detected in three independent melanomas was a C→T transition at codon 29 (CCT to TCT), resulting in a missense mutation, RAC1-P29S (FIG. 2A). Targeted sequencing of an additional 289 melanomas identified RAC1-P29S in 16 tumors. A similar frequency was observed among an independent cohort from Australia (4 of 94 melanoma cell lines). In total, RAC1-P29S was identified in 23 of 440 melanoma tumors and cell lines (5.2%, FIG. 14). There were no differences in RAC1-P29S frequency in primary and metastatic lesions (FIG. 14), suggesting this mutation occurs as an early event in development of melanoma. Consistent with this notion, the mutation was present in both the primary and metastatic tumors resected from one subject (patient coded YUTOGS, FIG. 14). Importantly, the RAC1-P29S was markedly more prevalent in males (8.0% in males versus 1.1% in females; p-value <0.001; FIG. 14). The mutation was not found in 11 congenital nevi from independent donors, although these nevi included four with BRAFV600E and three with NRAS mutations (two Q61K and one Q61R).

RAC1-P29S is somatic because it was not found in 11 cases for which germline samples were available, and is novel because it not present in public databases including dbSNP, 1,000 genomes and COSMIC, nor among 1,800 exomes of European subjects sequenced at Yale, nor by direct sequencing of 2,596 individuals from 57 anthropologically defined populations originating from diverse parts of the world.

The CCT to TCT change displays the C→T transition at a dipyrimidine site characteristic of UV induced mutations (Brash et al., 1991, Proc Natl Acad Sci USA, 88:10124). Transcription-coupled repair removes DNA photoproducts most efficiently on the template (transcribed) strand of expressed genes (Pleasance et al., 2010, Nature, 463:191). Therefore, the differences in C→T mutations in dipyrimidine sequences on the non-transcribed versus template strand of expressed and non-expressed genes were assessed. For expression, gene expression measurements in normal human melanocytes was used, as described earlier (Halaban et al., 2009, PLoS ONE, 4:e4563; Koga et al., 2009, Genome Res, 19:1462). For cutaneous melanomas, it was found that 2.7 times more mutations in the non-transcribed versus template strand of expressed genes, while this ratio was 1.1 for non-expressed genes. In sun-shielded melanomas, these ratios were 1.1 and 1.3, respectively, hinting that mutations in this class of melanomas are not due to a form of UV damage repairable by the nucleotide excision repair system. This shows that the C→T transition is located on the non-transcribed strand, where UV-generated cyclobutane dimers in expressed genes are not subject to transcription-coupled repair

(Pleasance et al., 2010, Nature, 463:191). Consistent with the role of sunlight in induction of this mutation, as well as in melanoma in general (Fears et al., 2011, Pigment Cell Melanoma Res, 24:574), all RAC1-P29S mutations originated in melanomas found in sun-exposed areas (head neck, limbs and upper trunk), with none in sun-shielded areas (acral, ocular, mucosal sites; FIG. 14, FIG. 1B).

The observed gender difference can be at least partly explained by differences in sun exposure, because there were 3-fold more melanoma lesions originating in the head/neck regions in men compared to women in this cohort (FIG. 2B). In contrast, this gender difference is unique to RAC1-P29S and was not observed for BRAF or NRAS mutations, which do not bear UV signature mutations. NRAS mutations are frequent in invasive (nodular) melanomas arising in chronic sundamaged skin (Lee et al., 2011, Br J Dermatol, 164:776; Edlundh-Rose et al., 2006, Melanoma Res, 16:471), whereas BRAF mutations also occur in sites that are not exposed to sun (Bauer et al., 2011, Pigment Cell Melanoma Res, 24: 345).

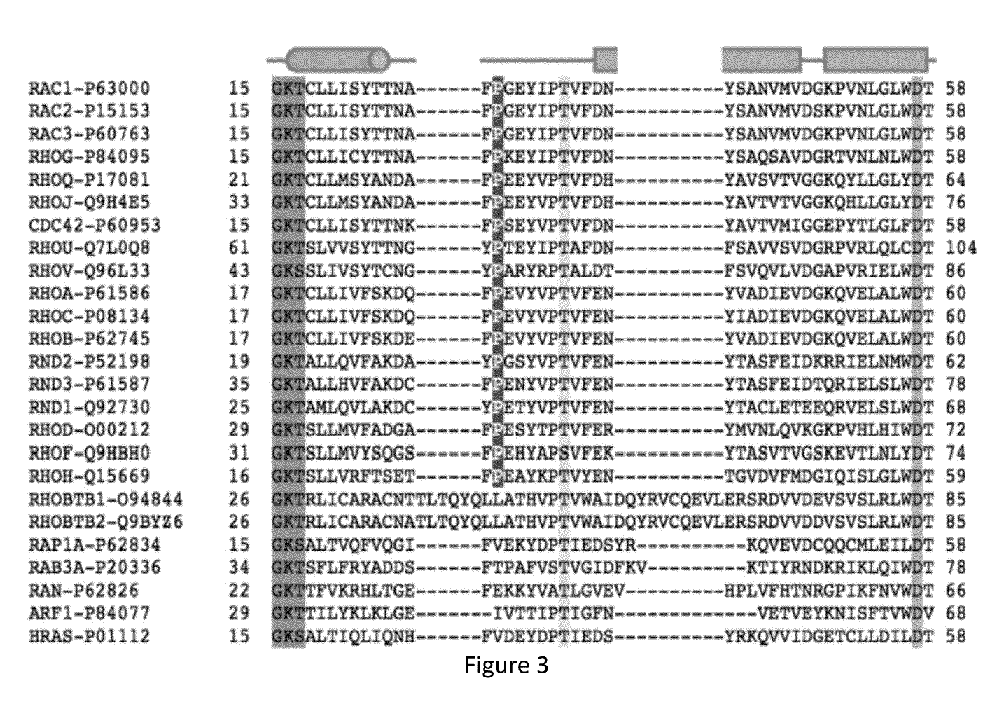

RAC1 is a member of the RHO family of small GTPases, which also includes CDC42. Structural studies have shown that the ‘switch I’ region, which contains the P29 mutation is a critical regulatory element of the GTPase superfamily and is important for nucleotide binding and interactions with effector molecules (Hakoshima et al., 2003, J Biochem, 134:327). A proline residue corresponding to RAC1 P29 is completely conserved in the RHO family of GTPases (with the exception of divergent RhoBTB1 and RhoBTB2) (Wennerberg et al., 2005, J Cell Sci, 118:843) (FIG. 3), and is located at the N-terminus of the switch I loop. Previous mutagenesis studies within the RAC1 switch I loop showed that mutation of the Pro29-Gly30 pair reduces the GTPase activity of RAC1 by 50% and results in increased effector activation (Menard et al., 1993, Biochemistry, 32:13357).

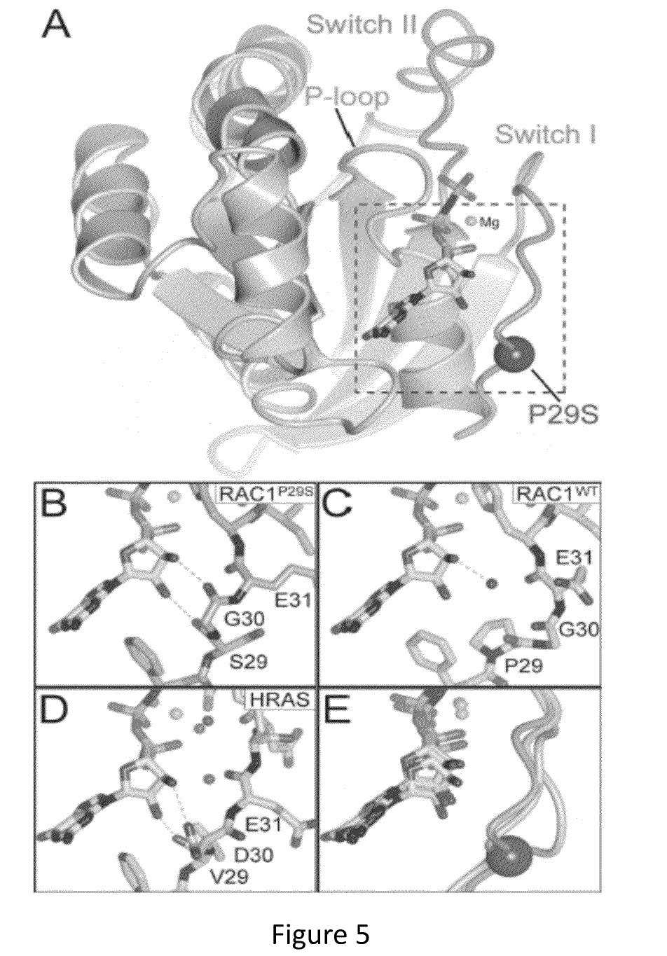

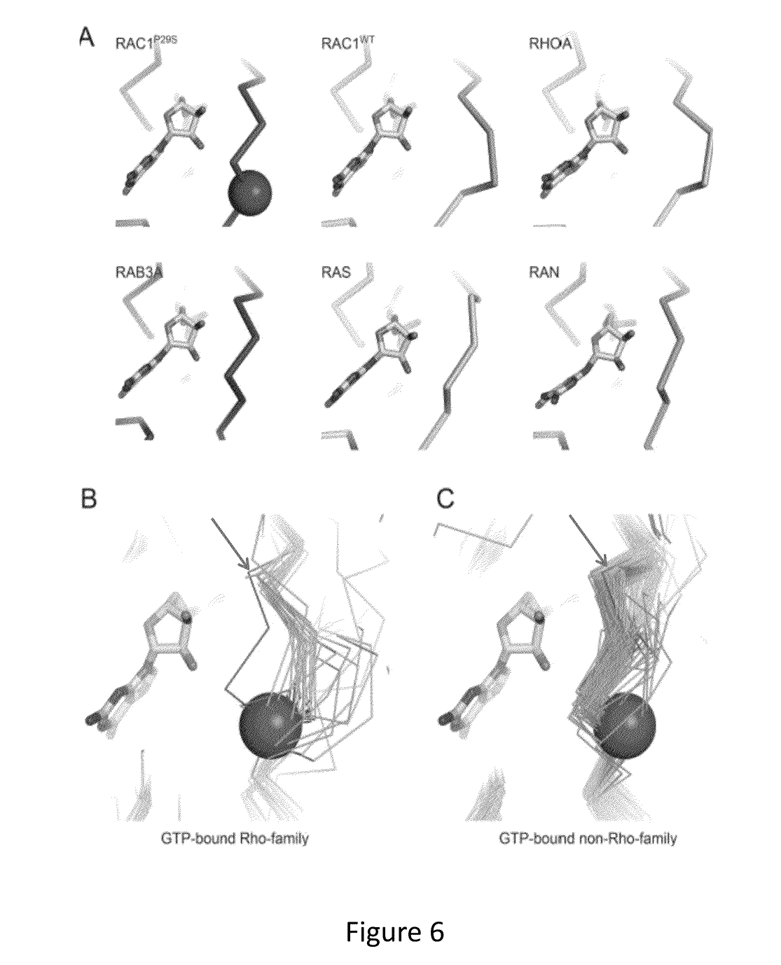

Two crystal structures of RAC1-P29S in complex with the slowly hydrolyzing GTP analogue, GMP-PNP, to 2.1 Å and 2.6 Å resolution, and the crystal structure of RAC1-WT to 2.3 Å resolution were determined (FIG. 5, FIG. 15 and FIG. 4). Interestingly, the crystal structure of RAC1-P29S is conformationally distinct from that usually observed in active-state RHO family GTPases and from RAC1-WT (FIG. 4, FIG. 5 and FIG. 6). In the active-state RAC1-P29S crystal structure, direct hydrogen bonds are observed between the ribose hydroxyl groups of GMP-PNP and the backbone carbonyls of both S29 and G30. This bonding contrasts with the pattern typically observed for RHO family GTPases, where water-mediated hydrogen bonds form between the ribose hydroxyl groups and switch I residues (Hakoshima et al., 2003, J Biochem, 134:327). Instead, it closely aligns to hydrogen bonding patterns observed in the crystal structure of activated HRAS, where direct ribose hydroxyl5 backbone interactions are commonly observed (FIG. 5 and FIG. 6). Although not wishing to be bound to any particular theory, the P29S mutation seems to release the conformational restraint inherent in a proline residue at position 29, therefore allowing a RAS-like altered conformation for GTP binding in the switch I loop and increased effector activation.

### Bioinformatics Analysis of Mutational Status of RAC1 Regulators and Signaling Partners

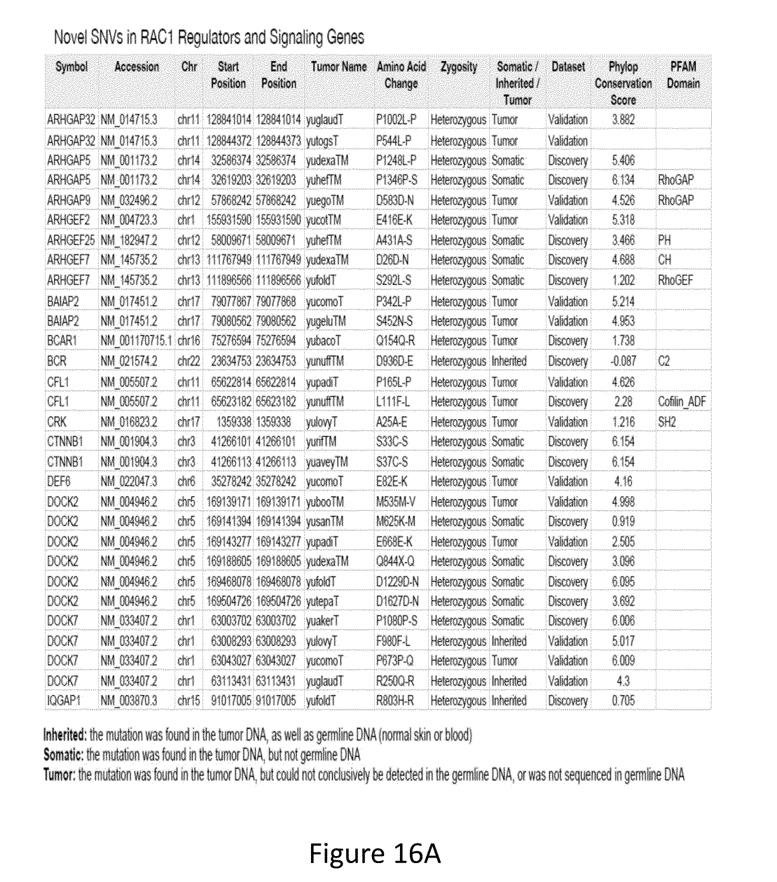

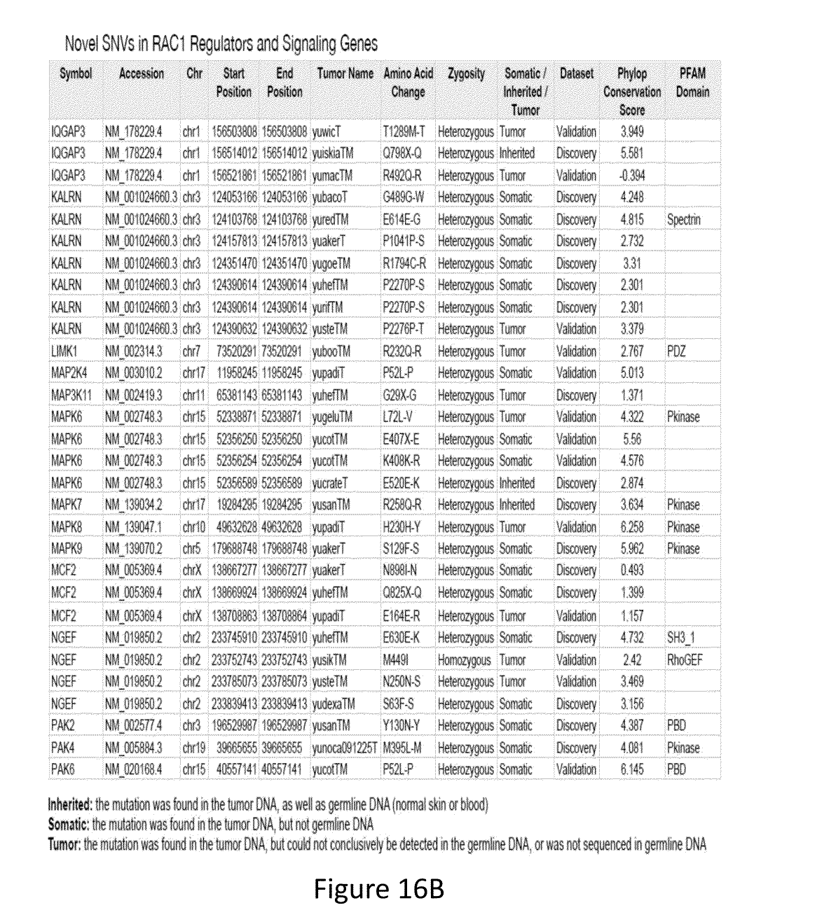

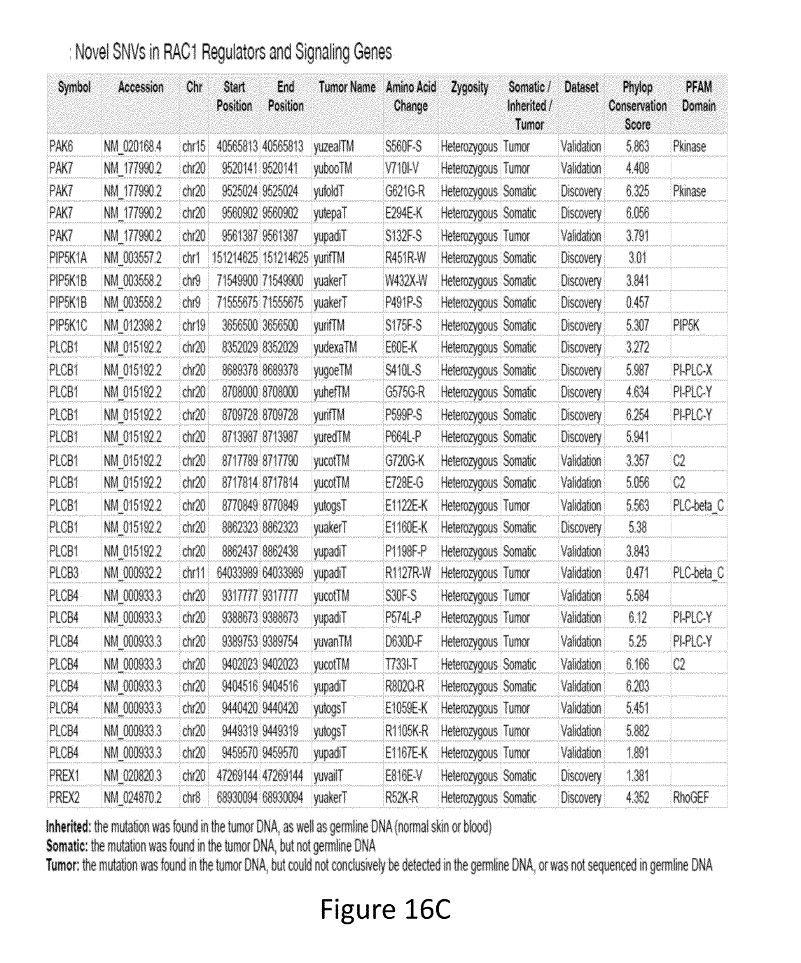

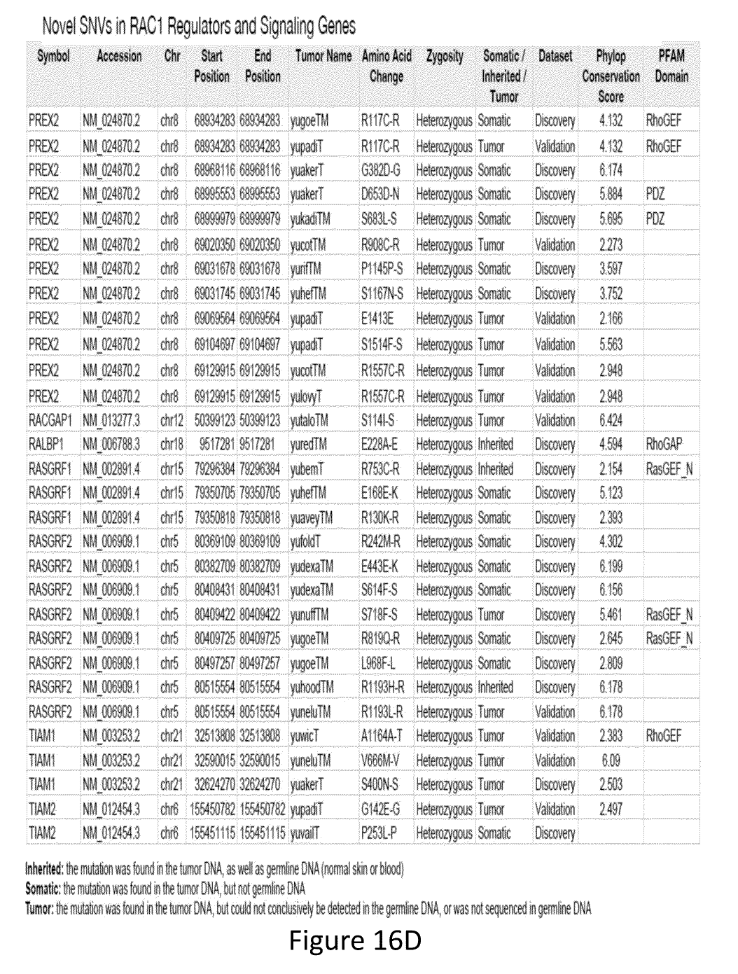

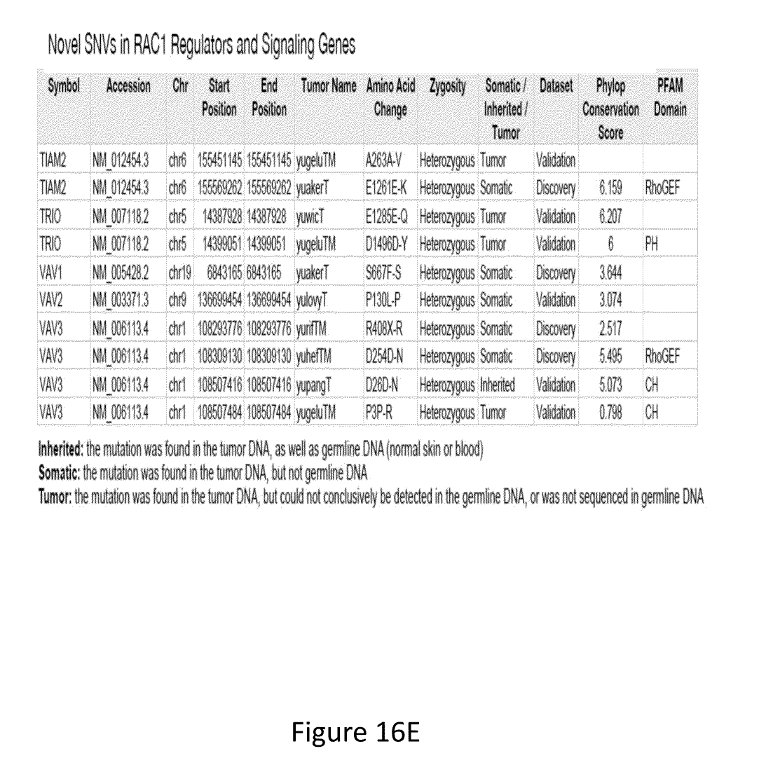

Genes from two pathways were downloaded from the NCI's Pathway Interaction Database (PID) (Schaefer et al., 2009, Nucleic Acids Res, 37:D674), “Regulation of RAC1 Activity” and “RAC1 signaling pathway,” generating two gene sets of size 31 and 24, respectively. It was checked whether any of these genes have mutations in these screens (FIG. 16). Particularly, genes with novel recurrent somatic variants across the discovery and validation screen (n=65), and genes harboring two or more distinct somatic variants (n=1,075), also known as CAN (Candidate Cancer) genes (Parsons et al., 2011, Science, 331:435) were examined Two genes with recurrent mutations, KALRN and PREX2, were presented in the Regulation of RAC1 Activity pathway, more than expected by chance (Fisher's exact test <p-value=0.008). In addition, a strong over representation of genes harboring two or more distinct mutations was found in that pathway, with a chi-square test p-value of 1.7×10⁻⁷. Equivalent tests were also statistically significant for genes harboring three and more somatic mutations (p-value 0.003), as well as four and more somatic mutations (p-value 0.002).

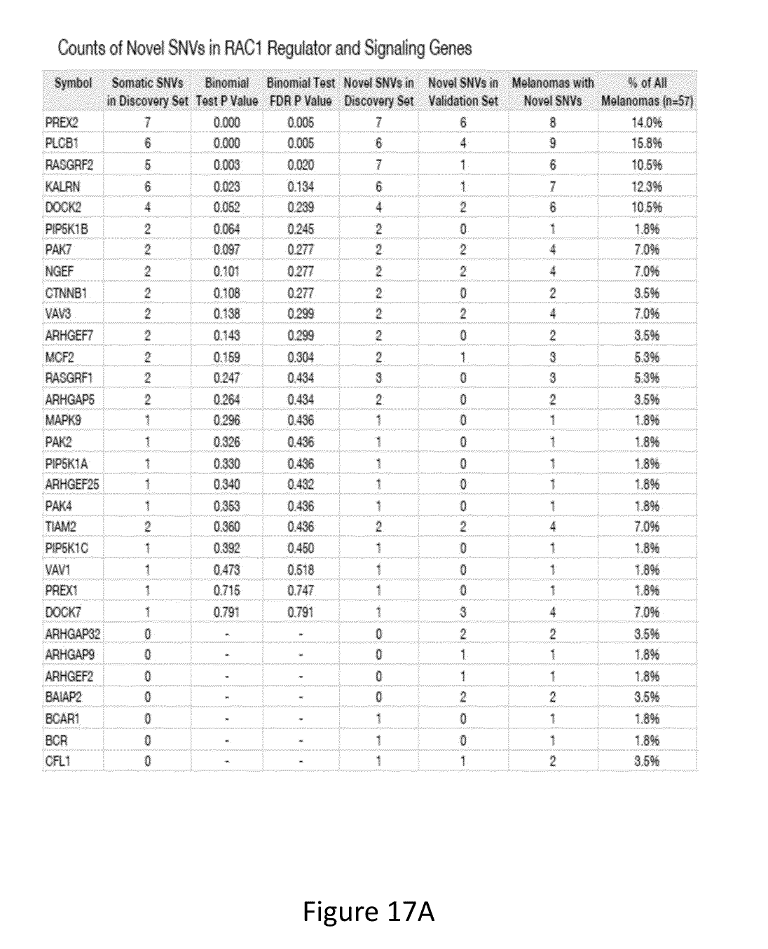

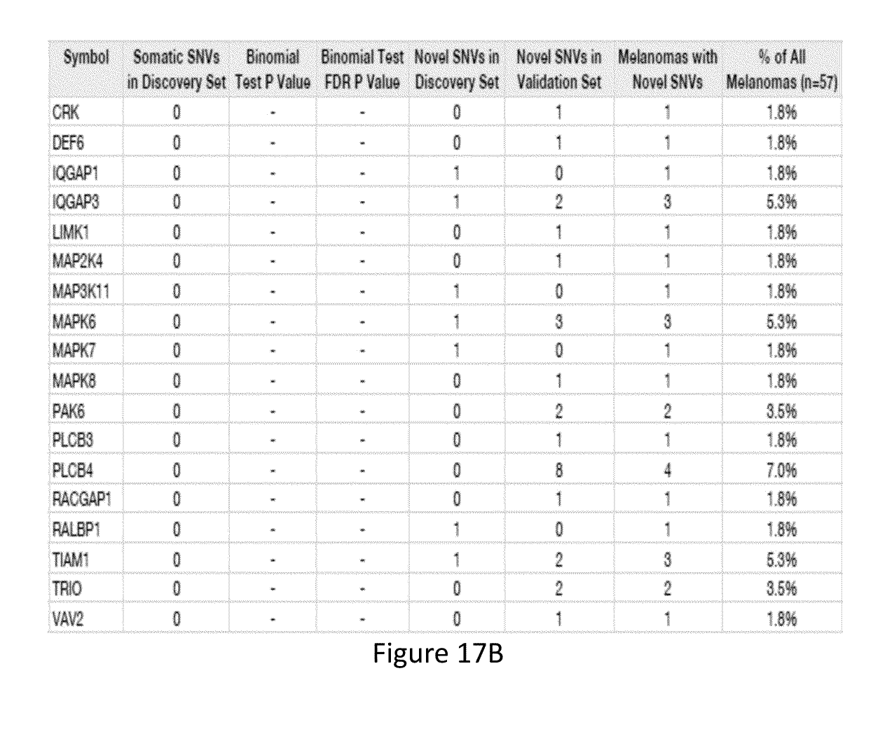

The results described above test for over-representations of genes with mutations in the two RAC1 pathways. It was also tested whether the number of observed mutations in genes from the RAC1 pathways occurred more often than would be expected by chance. Toward this end, the binomial test has been applied to measure the probability that the number of observed somatic mutations across genes can be explained by the tumor background mutation frequency. The background mutation frequency is composed of the mean number of somatic variants per sample, and is usually expressed as mutations per Mb. Given 6,034 observed somatic mutations over 27 samples, an exome capture area in the coding region of 24,152,296 bases, a background mutation frequency of 9.2 per Mb of a diploid genome was estimated. As this rate reflects the sum of all mutational properties across these specific melanoma samples, it is specific to the melanomas in this discovery set. Nevertheless, within this dataset, it is useful to identify deviation from the expected number of mutations per genomic region using the binomial test. The test was first applied to all genes in the two RAC1 PID pathways and genes identified by literature searches (FIG. 17). Altogether, PREX2, PLCB1, RASGRF2 and KALRN were identified as significantly enriched genes with somatic mutations (FIG. 17). This figure also lists novel SNVs in genes across both the discovery and validation sets related to the RAC1 pathway. For example, it shows that PREX2 variants occur in 14% of melanoma tumors in this cohort.

Finally, in order to identify additional critical RAC1 interaction partners, all novel genes with recurrent mutations were uploaded into GeneMania (Mostafavi et al., 2009, Genome Biol, 9 Suppl 1:S4), a protein interaction repository and analysis tool. Interactions between two sets of genes: TRAPP and P53 were observed, as well the interactions between RAC1 and KALRN and PREX2, already identified in the PID pathways. Interestingly, another gene, SLIT2, a known RAC1 inhibitor, was identified, raising the number genes with recurrent mutations and interaction with RAC1 to three.

### Functionality of RAC1-P29S Mutation

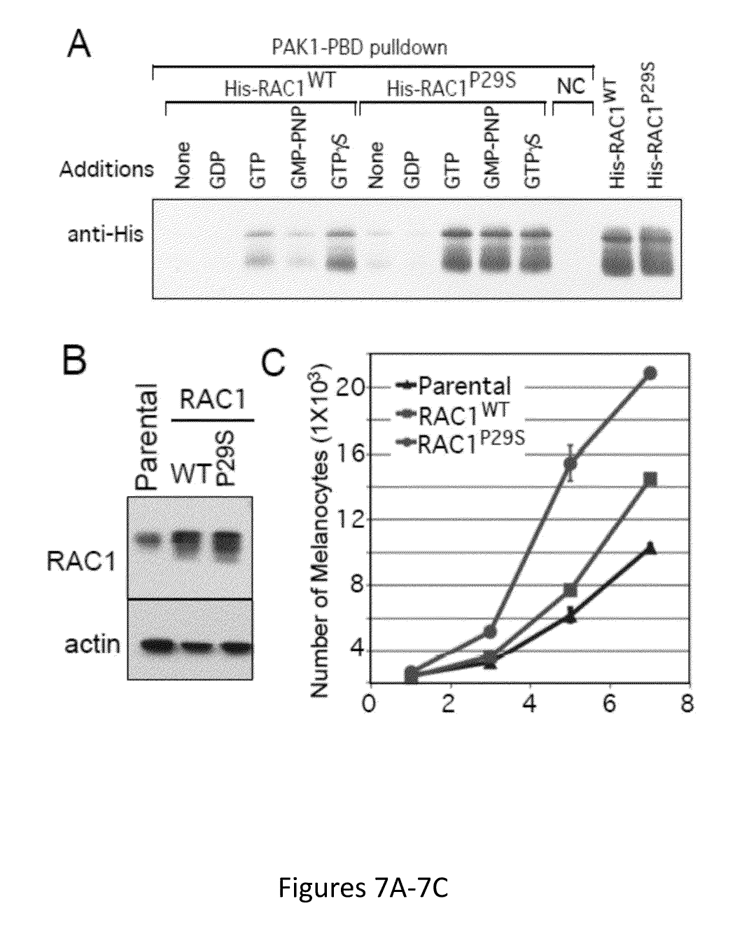

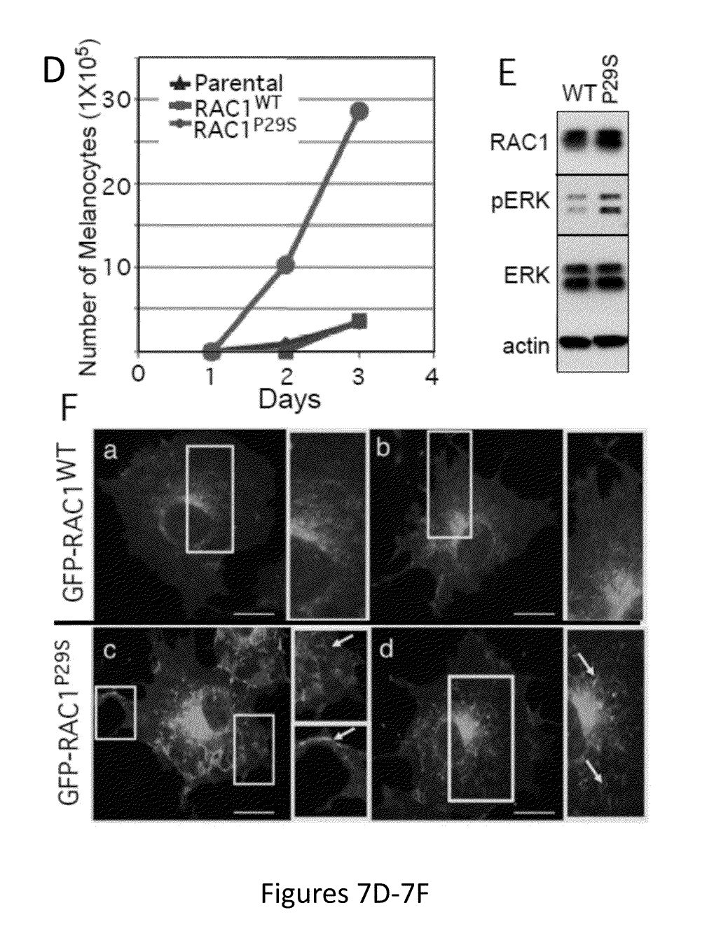

The effect of RAC1-P29S on effector binding and downstream activation was investigated by examining binding of purified recombinant His-tagged RAC1-P29S to GST-tagged PAK1-PBD (p21 binding domain), in the presence of GTP or slowly-hydrolyzable GTP analogs. Compared to RAC1-WT, GTP-loaded RAC1-P29S showed markedly increased binding to PAK1-PBD, similar to binding induced by GTPγS (FIG. 7A). This is consistent with either increased GTP binding or decreased GTP hydrolysis, as seen with other mutations altering P29. Similarly, expression of RAC1-P29S, but not RAC1-WT in normal mouse melanocytes, enhanced ERK phosphorylation, cell proliferation and migration (FIG. 7B-E). Lastly, GFP-tagged RAC1-P29S, but not RAC1-WT, induced strong membrane ruffling and the protein accumulated in ruffling membranes of COS-7 cells (FIG. 7F), a hallmark of an activated protein (Ridley et al., 1992, Cell, 70:401). These findings confirm that the RAC1-P29S mutation is gain of function and activates downstream signaling.

The data presented herein identify RAC1-P29S as a recurrent UV signature mutation in 5% of melanomas. The mutation confers increased effector binding to this small GTPase, provides proliferative and migratory advantage to normal melanocytes, and induces membrane ruffling. Bioinformatics analysis of the sequencing data presented herein suggests enrichment of mutations in upstream regulators and effectors of RAC1, raising the possibility that RAC1 signaling plays a role in additional melanomas (FIGS. 16 and 17). These findings suggest that inhibitors of RAC1 or its effectors might be of therapeutic benefit in melanomas.

### Example 2

### Novel Driver Mutations and Targetable Protein Kinases in Melanomas

Identification of mutations in receptor and non-receptor protein kinases in cancer cells that drive malignant transformation (“driver mutations”) have revolutionized patient care by opening the door to patient-tailored targeted therapies that improve survival and extend patients' life spans.

The materials and methods for these experiments are now described.

### Melanoma Specimens

Melanoma tumors that fulfill some of the following criteria were selected for sequencing: a) snap frozen tumors composed of at least 80% tumor cells; b) tumors with matched short-term melanoma cells in cultures for functional validation (up to passage 10); c) melanomas for which there is peripheral blood and/or adjacent skin; d) metastatic melanoma for which there is access to the primary lesion in paraffin blocks to assess the presence of mutations in early stages of the disease. Melanomas from different stages, different locations, and different types (cutaneous, mucosal, acral) were used.

All tumors are routinely sequenced for melanoma-specific mutations, such as BRAF, NRAS, CDKN2A and KIT (when applicable) by the YSPORE Specimens Core. In addition, associated patient characteristics are available, including history of sun exposure, familial cancer, demographics and clinical and pathological information, and can be related to the kind of mutation revealed by sequencing.

### High Throughput Sequencing

Based upon a preliminary calculation using the available subset of paired samples, the prevalence of these kinase mutations are as follows: per sample, approximately 342 (in sun-exposed melanomas) vs. 28 (in non-sun-exposed melanomas) somatic mutations that cause an amino acid change are found, with ˜6 of those mutations falling into a protein with kinase function. Unlike BRAF or NRAS, the novel mutations are spread in different locations of critical domains. Thus, a list of kinases that show somatic hits among the melanoma samples, and other related genes, such as kinase regulatory subunits, that accumulate somatic mutations can be composed. The list of genes is then interrogated using a custom-made exome capture array across melanomas with areas of 80% invasive tumor identified. The custom-made array contains the exons of all the presently identified mutant genes. Exome-capture provides information on mutations in expressed and non-expressed genes.

In this procedure, genomic DNA is fragmented to about 300 bp, ligated with PCR-based oligonucleotide barcodes for sample indexing, captured by hybridization in solution to custom-designed oligonucleotide baits, and amplified libraries are pair-end 100 bp sequenced on the Illumina Genome Analyzer II. Barcoding the samples allows sequencing of at least 12 samples of DNA per flow cell (paired-end) with high number of mean sequence coverage (>150) for roughly 500 target genes. Sequencing is applied to tumor and matched germline DNA to identify somatic variants. The sequencing reads are first mapped to the reference genome, subjected to a pile-up procedure, followed by the variant call. Variants are then prioritized as described elsewhere herein. Several companies provide in-solution custom capture arrays such as Roche, NimbleGen, and Agilent (SureSelect Target). Mutations are validated by Sanger sequencing to rule out false positives.

A mutation screening process was used to identify unique deleterious missense and nonsense mutations that potentially impact RNA stability and protein function. Briefly, sequencing data undergo intensive bioinformatic analysis at different steps. They are compared to known gene variants, variants reported in the 1,000 Genomes Project and dbSNP database to screen out common polymorphisms in the human population. In addition, identified nucleotide variations are compared to the database of 350 individuals currently being sequenced (exome-capture). Candidate variants are categorized by gene location, predicted effect (frameshift, in-frame insertion or deletion, synonymous substitution, nonsynonymous substitution, splice site alteration, or nonsense). Functional importance scores are assigned based on conserved regions and functional domains of the protein, such as cSNP (Cerami et al., 2010, PLoS One 5:e8918), as well as based on previously determined x-ray crystal structures, when available. Mutations that are already listed in COSMIC are identified. Further, driver genes that accumulate a larger number of somatic variants than expected according to previously published methods, calculating the gene selection pressure (Greenman et al., 2007, Nature, 446:153-8) and the cancer mutation prevalence (CaMP) score (Wood et al., 2007, Science, 318:1109-1113) are identified. Gene Ontology (GO) annotations and PubMed literature association of driver genes are analyzed to determine the cellular pathways that might be disturbed and provide druggable targets. Another approach for driver gene selection involves data integration from other omics modalities. These include: 1) whole-genome expression data of about 40 different melanoma cells and normal human melanocytes and nevocytes (Halaban et al., 2009, PLoS ONE 4:e4563) to assess mutant gene expression in melanoma cells; 2) genomic aberrations (amplification/deletions) derived from SNP/CNV analysis employing the Illumina 1,000 SNP arrays on 35 metastatic melanoma freshly isolated from tumors to determine connection to genomic aberrations; 3) tyrosine, and/or Ser/Thr phosphorylated proteins identified by phosphoproteomic m analyses performed in collaboration with Cell Signaling Technology, Inc. (CST). These data provide information on the activated status of protein kinases and signal transducers in snap-frozen melanoma tumor specimens and melanoma cells.

The results of the experiments are now described.

### Novel Melanoma Associated Mutant Kinases

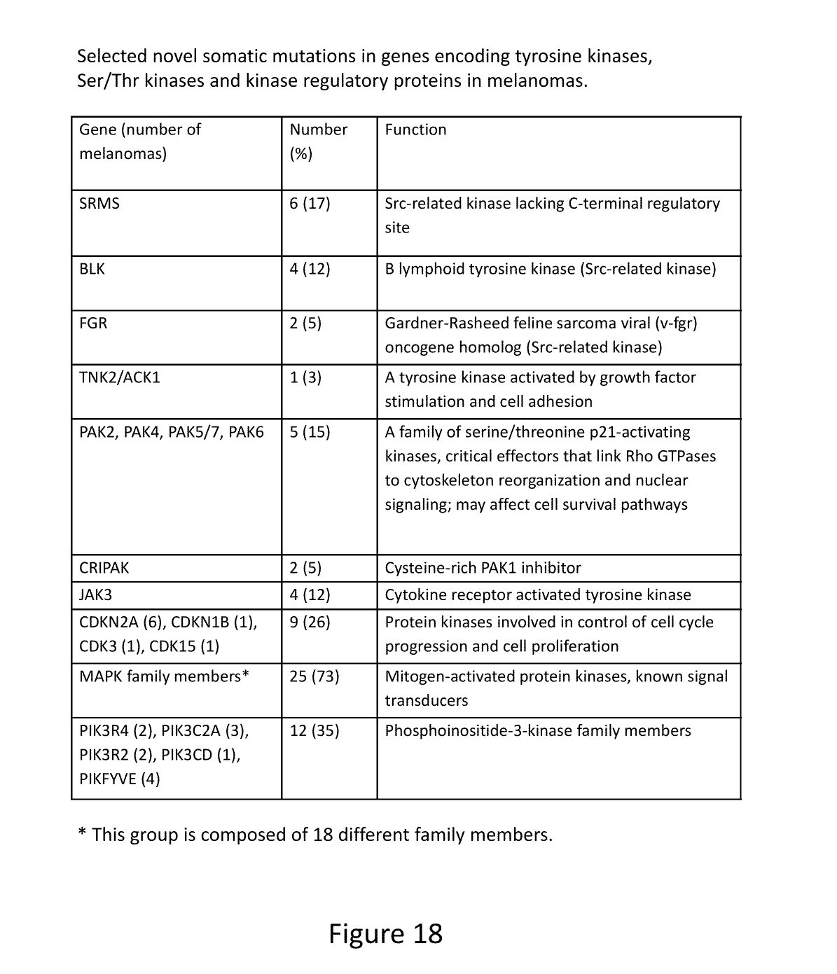

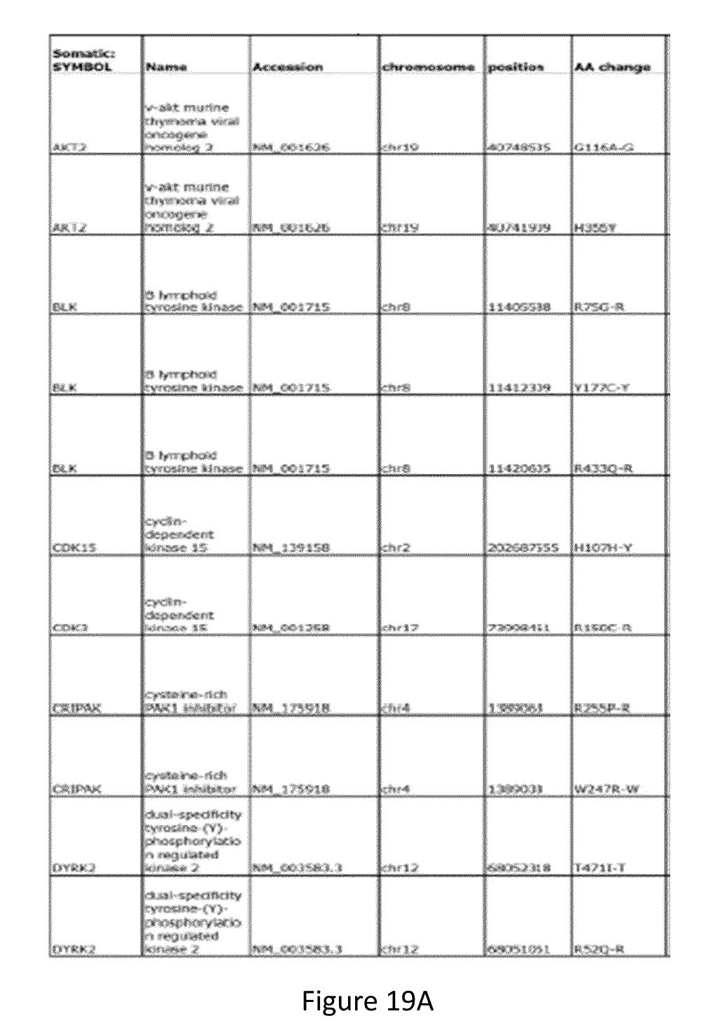

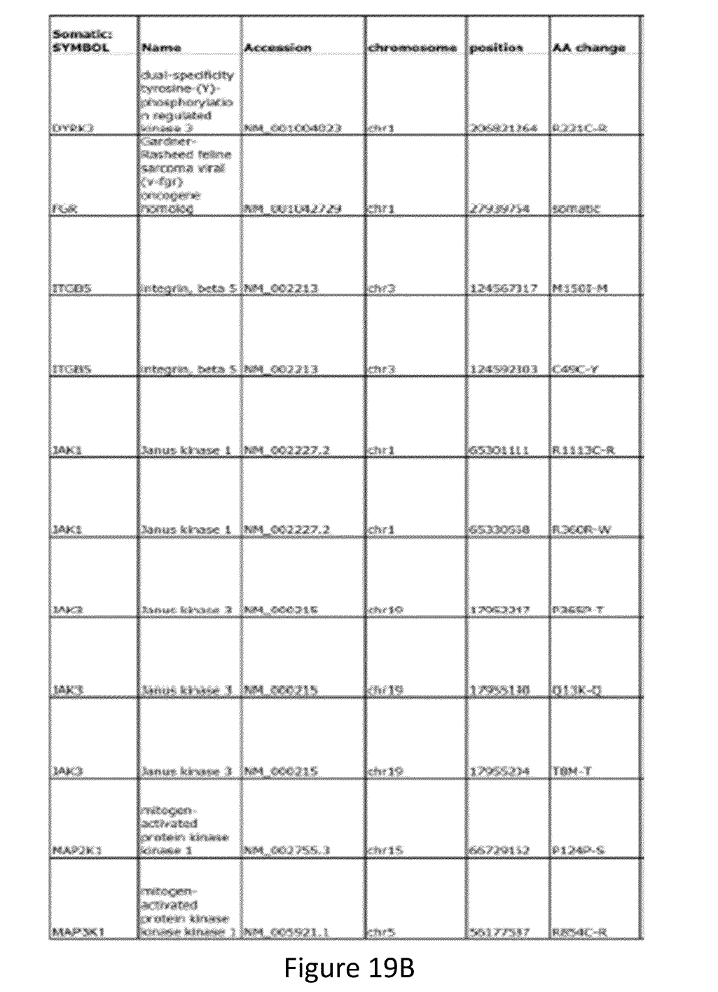

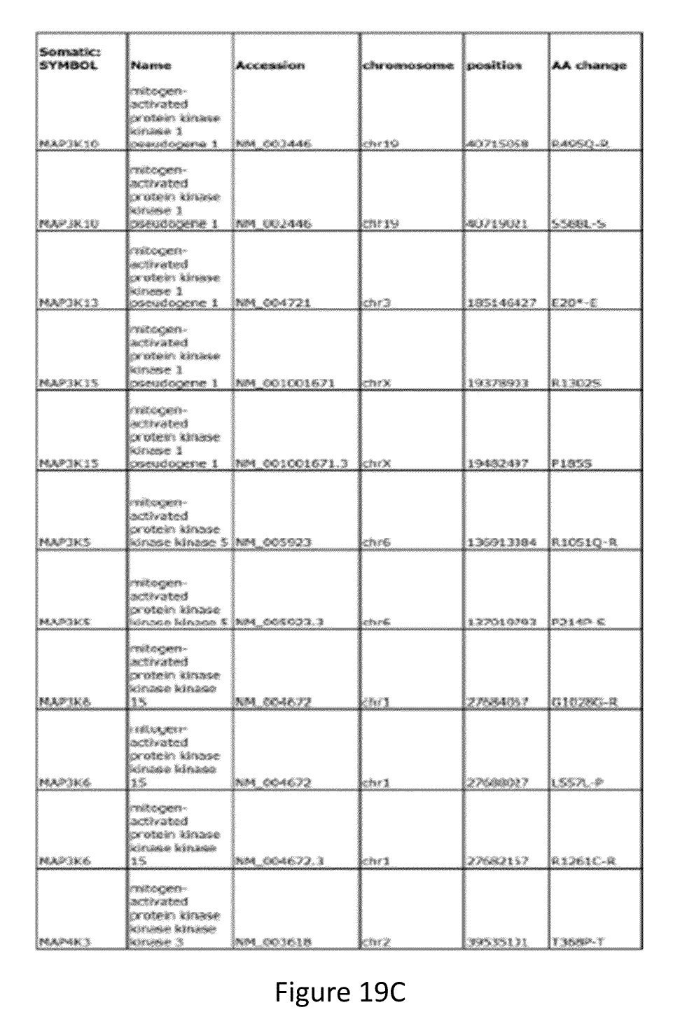

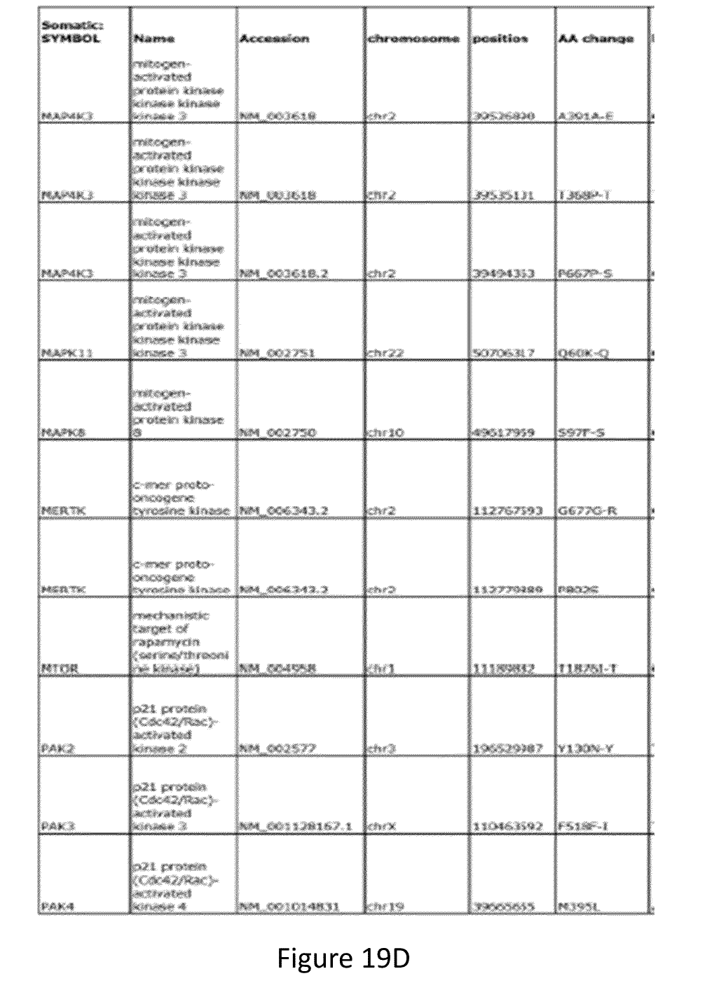

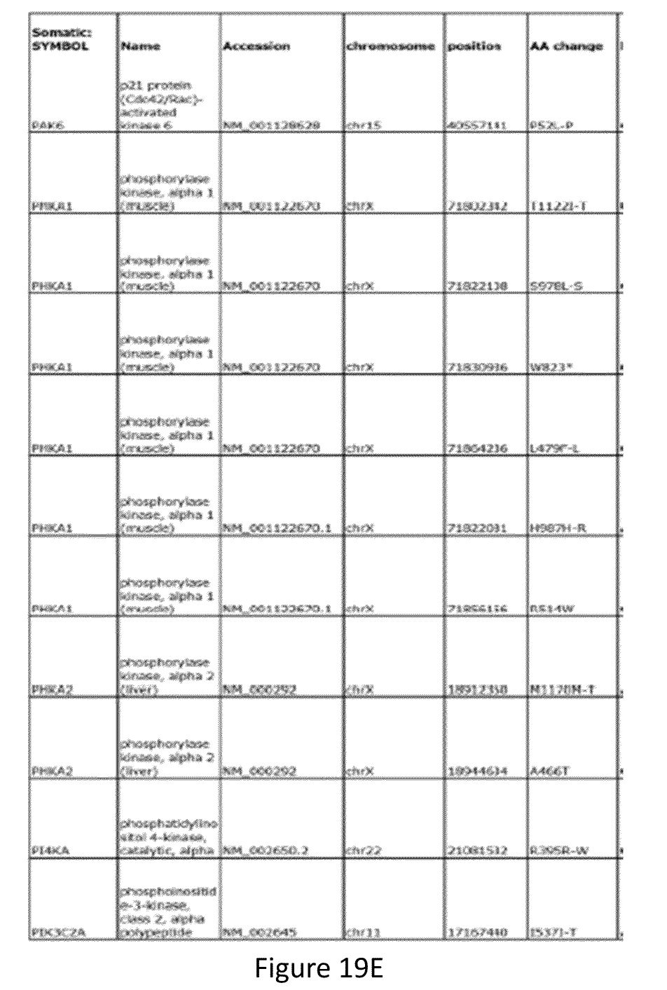

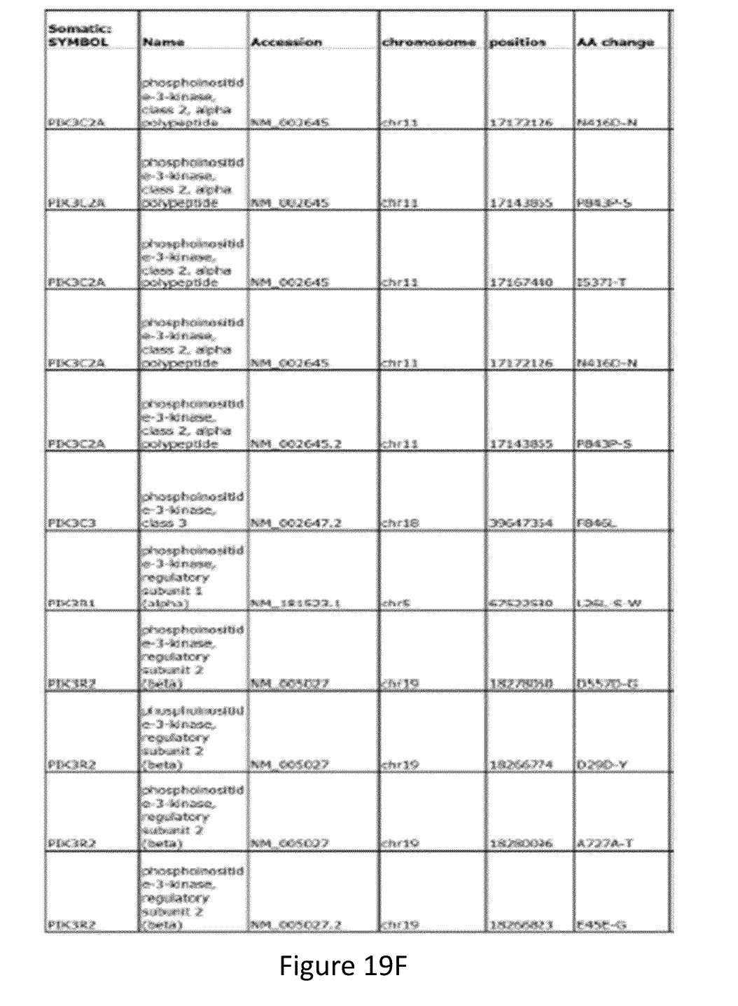

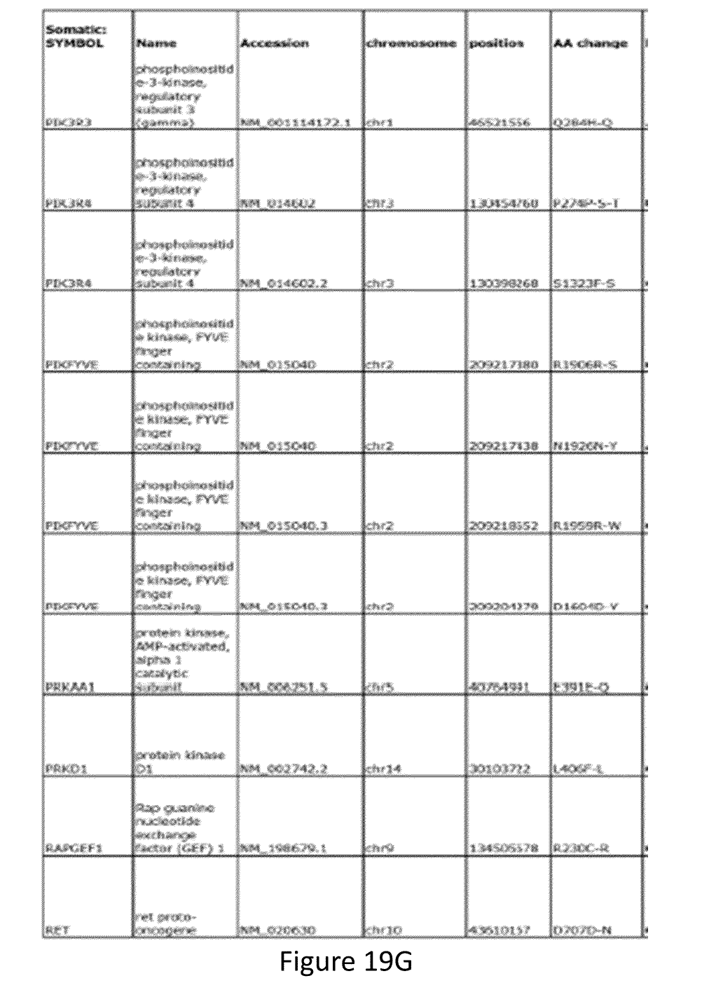

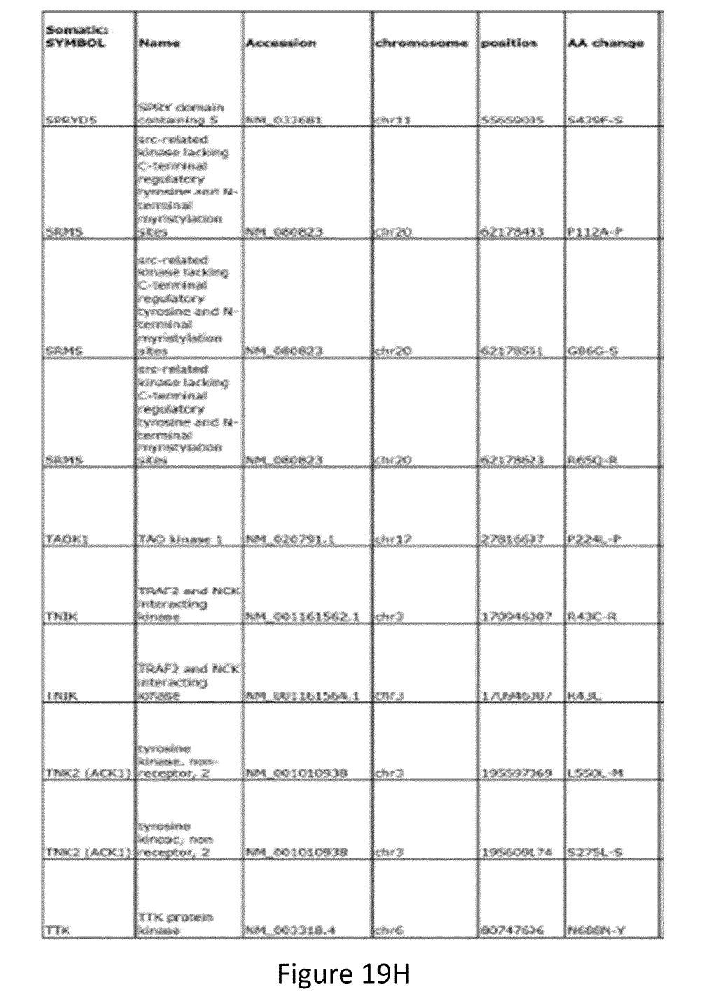

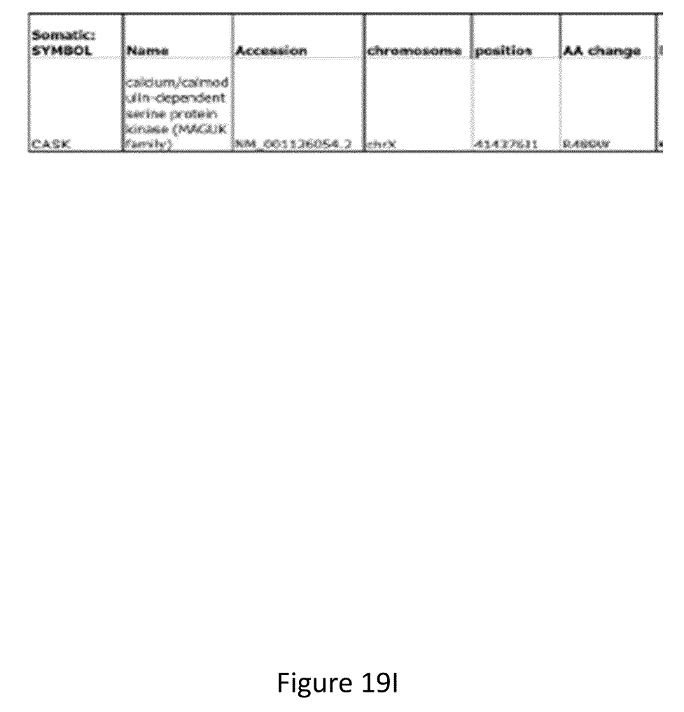

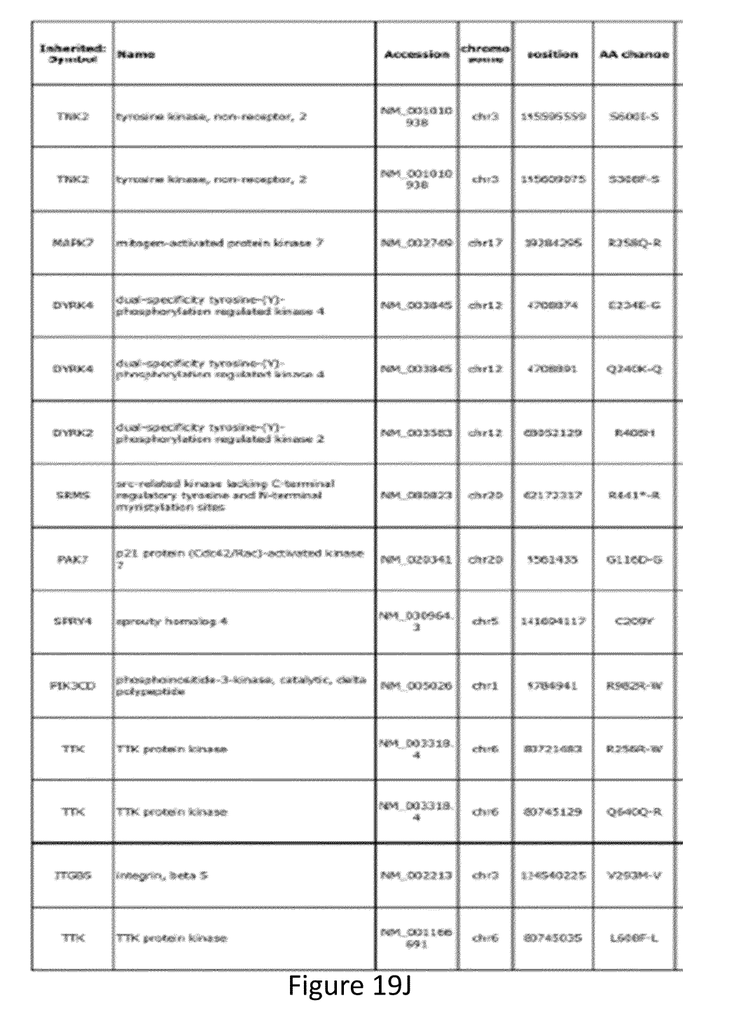

In order to discover new targets for therapy, the coding regions of 57 melanomas were sequenced and somatic mutations and inherited Single Nucleotide Variants (SNVs) were identified in several protein kinases and regulatory proteins (see FIGS. 18 and 19). The sequencing of 57 melanomas showed marked variations in mutation frequencies of genes encoding protein kinases among individual tumors (FIG. 18). Several somatic mutations in the Src-related kinases, SRMS, BLK and FGR, were identified. Additional mutations found in proteins known to be oncogenic, include CDKs (Rizzolio et al., 2010, Curr Drug Targets, 11:279-90), JAK3 (Zhou et al, 2001, Mol Cell, 8:959-69; Walters et al., 2006, Cancer Cell, 10:65-75), PIK and MAPK family members, some of which are targeted by currently used drugs, and in the kinase domains of kinases (ACK1, PAK-4 and PAK5/7). Initial results show that mutations in ACK1, CDKs and PAKs are mutually exclusive. In addition, none of the melanoma cell lines with PAK mutations carried the BRAF activating mutations.

Sequencing shows that the inherited ACK1-S522I single nucleotide variant (SNV) is highly prevalent across the sequenced melanoma samples (5%). Other SNVs are also in the germline DNA, such as ACK1-S245, PAK5/7-G116D, and MAPK3-R258Q. These melanoma SNVs have not previously been detected in healthy individuals.

### Example 3

### Src Kinase Mutations

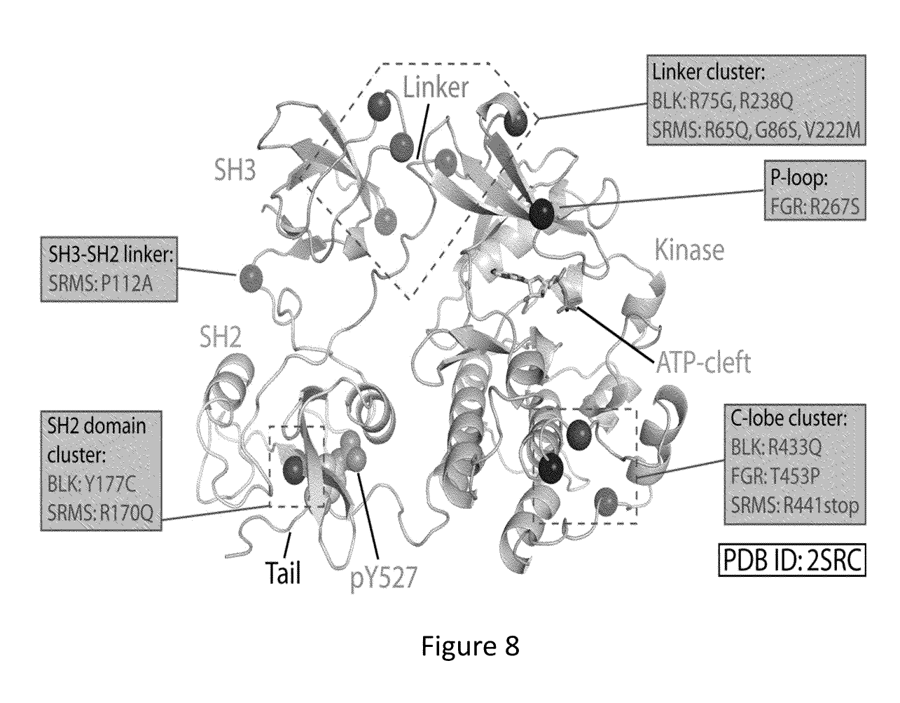

Although there are no crystal structures of any of these Src-related kinases, Src kinase has high homology to SRMS, FGR and BLK proteins, with 43% identity and 58% similarity to SRMS, 67% identity and 78% similarity to FGR, and 62% identity and 76% similarity to BLK. It is therefore expected for these proteins to share global domain structure and regulation with Src kinases and initial structural analysis has been conducted. All of these proteins contain an N-terminal SH3 domain, followed by a SH2 domain, a linker and a kinase domain. The locations of the novel point mutations were mapped onto the crystal structure of autoinhibited Src kinase (PDB ID: 2SRC) (Xu et al., 1999, Mol Cell, 3:629-38) (FIG. 8). Src-like kinases are regulated by a ‘snap-lock’ mechanism (Boggon et al., 2004, Oncogene, 23:7918-27), whereby the SH3 and SH2 domains stack against the kinase domain via a linker region between the SH2 and kinase domains, locking the kinase domain in an inactive state. On mapping the locations of these mutations onto the structure of autoinhibited Src kinase it is found that the mutations cluster at specific regions important for the activity and regulation of Src-related kinases. First, there is a cluster of mutations around the linker between the SH2 and kinase domains, mutations in this region have been shown to destabilize the autoinhibited conformation and activate the kinase (Briggs et al., 1999, J Biol Chem, 274:26579-83). Second, there is a cluster of mutations in the SH2 domain that are likely to disrupt the autoinhibited conformation by reducing or abrogating the ability to bind the phosphorylated tail (pY527 in Src). Third, there is a mutation in SRMS in the linker region that connects between the SH3 and SH2 domains. Mutations in this region have previously been shown to alter conformational dynamics between the domains and activate Src-like kinases (Young et al., 2001, Cell, 105:115-26). Fourth, there is an FGR mutation in the glycine-rich P-loop of the kinase, potentially important for enzyme kinetics. Fifth, there is a cluster of mutations in the C-lobe of the kinase that could potentially impact substrate binding. Overall these mutations tend to correlate well with deregulation of Src-family kinases, and may implicate changes in Src-related kinase signaling pathways for patients with these mutations.

### Structure-Function Analyses

Detailed biochemical analysis is performed to determine the impact of the newly identified mutations on enzymatic activity and other properties of the kinases found to be mutated in the melanomas. Further, the activity, cellular localization and stability is compared of wild type or mutant kinases ectopically expressed in transfected cells or in cell lines derived from the original tumors. The oncogenic activity of mutant kinases is explored by testing their impact on growth in-vitro in soft agar, formation of cell foci and tumors in murine model systems, compared to the common normal phenotype. Although the mechanism of activation of Src kinases is well understood, the X-ray crystal structures of one of these kinases in an autoinhibited conformation is determined to better understand the atomic-level basis for activation. The goal is to validate the oncogenic capacity of the newly discovered mutated kinases in order to set the stage for discovering novel therapeutic agents that will interfere with their kinase activity.

### Impact on Proliferation and Tumorigenic Functions

The consequences of silencing or overexpression of normal and mutant kinases in melanoma cells is explored in order to reveal the role they play in melanoma cell growth, invasion and migration, employing standard transfection approaches and assays known in the art. Oligonucleotide siRNA or lentivirus shRNA is used to suppress the levels of the tyrosine kinase in cells expressing normal or mutant protein, or were shown to express constitutively activated kinase (as indicated in phosphoproteomic studies). The mutant proteins are generated by site directed mutagenesis. The effect of mutant kinase expression or silencing on activation of other cell signaling molecules such as MAPK or AKT are explored, employing standard immunoblotting analysis and kinase assays as previously described (Halaban et al., 2010, Pigment Cell Melanoma Res, 23:190-200).

### Inhibitor Studies

The effect of PLX4032, Dasatinib, and other Src-kinase inhibitors on the proliferation, cell foci formation, and growth in soft agar of melanoma cells harboring either wild type or mutant kinase is explored. Cell proliferation and cell viability is tested using the Coulter counter and CellTiter glow, respectively. In addition, novel drugs developed by Plexxikon, Inc. and other companies is explored to test their efficacy in inhibiting melanoma cells harboring these newly discovered mutations in Src-related kinases.

### Example 4

### Mutations in PAK Family of Kinases

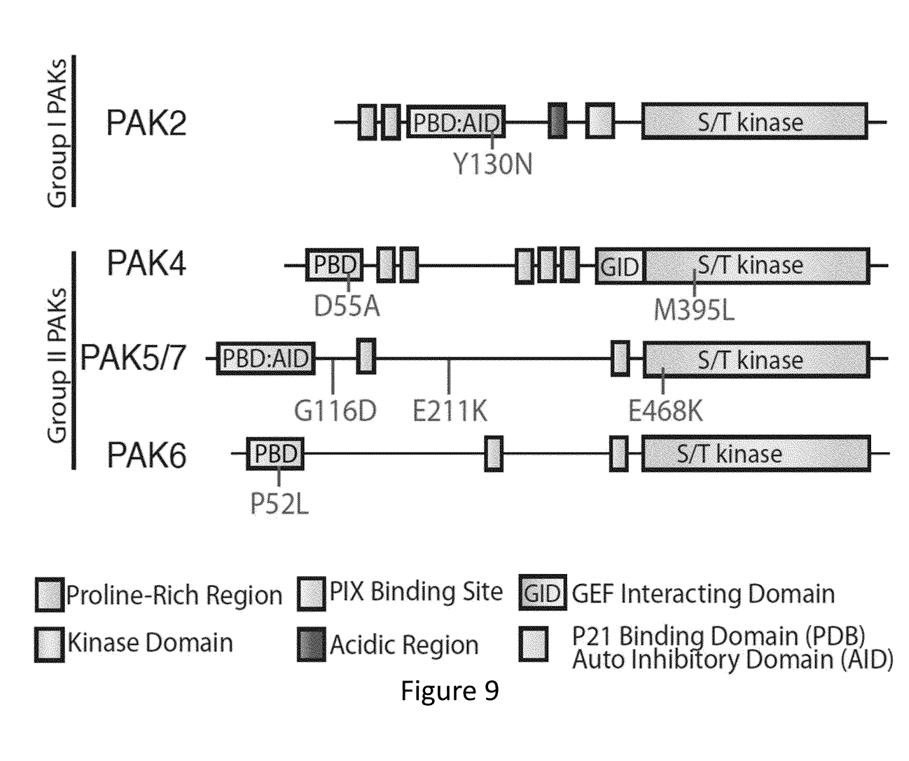

Mutations/SNV in four members of the PAK family, PAK2, PAK-4, PAK5/7 and PAK6 (FIG. 9) and in CRIPAK, an inhibitor of PAK1 (Talukder et al., 2006, Oncogene, 25:1311-19) were identified as described elsewhere herein. In this group only PAK5/7 G116D is an inherited variation but no data are available on the frequency of this variant in normal populations. PAK family members are recognized as having major roles in oncogenic processes and thus are good candidates for targeted cancer therapy (Murray et al., 2010, Proc Natl Acad Sci USA, 107:9446-51). The p21-activated kinases (PAK) family is organized into two groups with different regulation mechanisms and functions (Arias-Romero et al., 2009, Biol Cell, 100(2): 97-108), group I PAKs include PAKs 1-3 and group II include PAKs 4, 5/7 and 6. The group I PAKs are activated by binding to GTP-bound CDC42 or Rac.

These kinases activate MAP kinase pathways and regulate cytoskeletal actin assembly, and their dysregulation is associated with cell adhesion and metastasis in cancer (Kong et al, 2009, J Biochem, 146:417-27; Chandramouli et al, 2007, Carcinogenesis, 28:2028-35; Eswaran et al., 2009, Cancer Metastasis Rev, 28:209-217; Wells et al., 2010, Biochem J, 425:465-73; Molli et al., 2009, Oncogene, 28:2545-55; Kumar et al., 2006, Nat Rev Cancer, 6:459-71). PAK1 promotes cell proliferation through phosphorylation of RAF1 (Ser338) and MEK (Ser298), two components of the MAPK cell-signaling pathway. Therefore, constitutively active PAKs can promote not only cell proliferation but may also interfere with targeted therapy, such as BRAF kinase inhibitors. Like group I PAKs, the group II PAKs are important for cell motility and survival. The recent efforts of pharmaceutical companies, such as Pfizer, to develop drugs that inhibit this family of kinases (see for example PF-3758309 for PAK-4 (Murray et al., 2010, Proc Natl Acad Sci USA, 107:9446-51)), provide an incentive to study these family members in melanomas. The crystal structure of PAK1 in complex with its regulatory CRIB (PBD:AID) domain (Lei et al., 2000, Cell, 102:387-97), and the sequence identity of PAK1 and PAK2 in this region, suggests conserved mechanisms of action. It is predicted that binding of GTPase to the CRIB domain will sequester this domain away from the kinase in an activation step (Lei et al., 2000, Cell, 102(3): 387-97; Eswaran et al., 2007, Structure, 15(2): 201-13; Pirruccello et al., 2006, J Mol Biol, 361(2): 312-26).

The structural basis for regulation of group II PAKs has not been described crystallographically and the molecular basis for CRIPAK inhibition of PAK1 is not known. Therefore a structure-directed functional study is done to investigate the effects of melanoma-associated mutations within group I PAKs, and the normal and dysregulated mechanisms of regulation for group II PAKs. A structure-guided approach to understand the roles of PAK-family dysregulating mutations will facilitate better PAK-targeted therapeutics.

### Structure-Function Analysis

A broad and wide-ranging investigation is done to validate the functional role of each of the PAK family members. The effect of known inhibitors, such as the ATP-competitive inhibitor PF-3758309 identified for PAK-4 (Murray et al., 2010, Proc Natl Acad Sci USA, 107(20): 9446-51), on melanoma cells harboring PAK-4 mutations is tested. The effect of PAK overexpression and silencing at the cellular level and on downstream signaling molecules (e.g. MEK or RAF) is investigated by standard immunoblotting techniques. These studies provide information on the nature of PAK pathways controlling melanoma cell growth and survival.

Novel Therapeutic Agents that Block the Activity of Oncogenic PAK Kinases

PF-3758309 is known to inhibit only PAK-4. A rational structure-guided approach is used to target mutant oncogenic kinases with small molecule inhibitors. The kinase domains of the PAK family members are readily available for structural studies (Murray et al., 2010, Proc Natl Acad Sci USA, 107:9446-51; Eswaran et al., 2007, Structure, 15:201-13; Yi et al., 2010, Biochem Pharmacol, 80:683-9; Maksimoska et al., 2008, J Am Chem Soc, 130:15764-5; Lei et al, 2005, Structure, 13:769-78). The PDB contains structures for the kinase domains of PAK1, PAK-4, PAK5/7 and PAK6 and can be expressed in E. coli. Purified, crystallization-quality wild type and mutant PAK kinase domains are generated to investigate the potential of targeted inhibition of these proteins in the melanoma setting, and specifically targeted inhibition of validated mutant kinase. High-throughput screening of potential inhibitors is performed by screening inhibitors similar in structure to PF-3758309 and inhibitors of other kinase activity that show increased potency when targeted at mutant PAK family members, or with selectivity for group I or group II PAKs. Structural studies enable in silico drug discovery and provide a framework for rational structure-directed drug discovery of lead compounds.

Compounds with preferential inhibition of the mutated forms of the PAKs in comparison to wild-type forms that inhibit the kinase activity at nanomolar concentrations are chosen. These compounds are tested on PAK-mutant and wild type melanoma cells to determine relative inhibition of cell growth and colony formation. The best compounds are tested in animal xerographs models.

### Example 5

### Inherited Mutations in ACK1

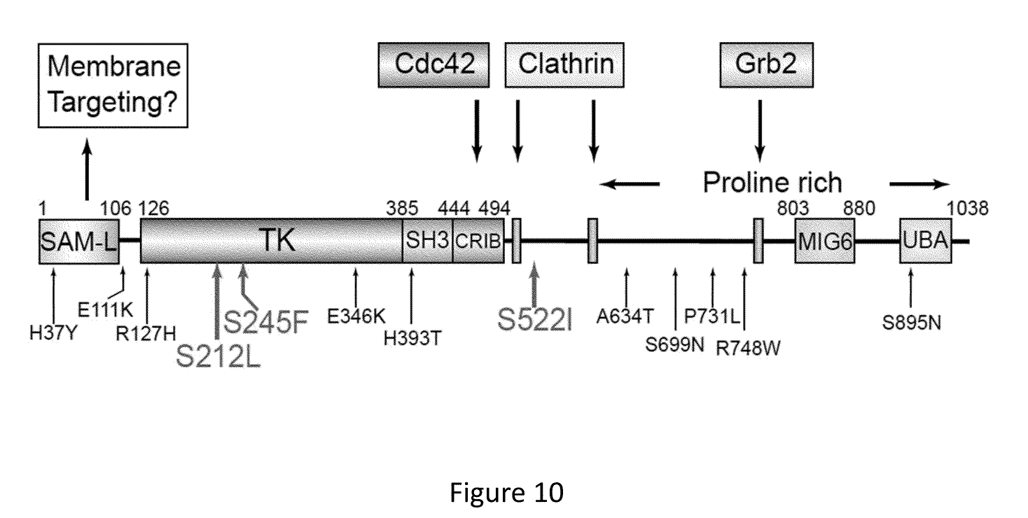

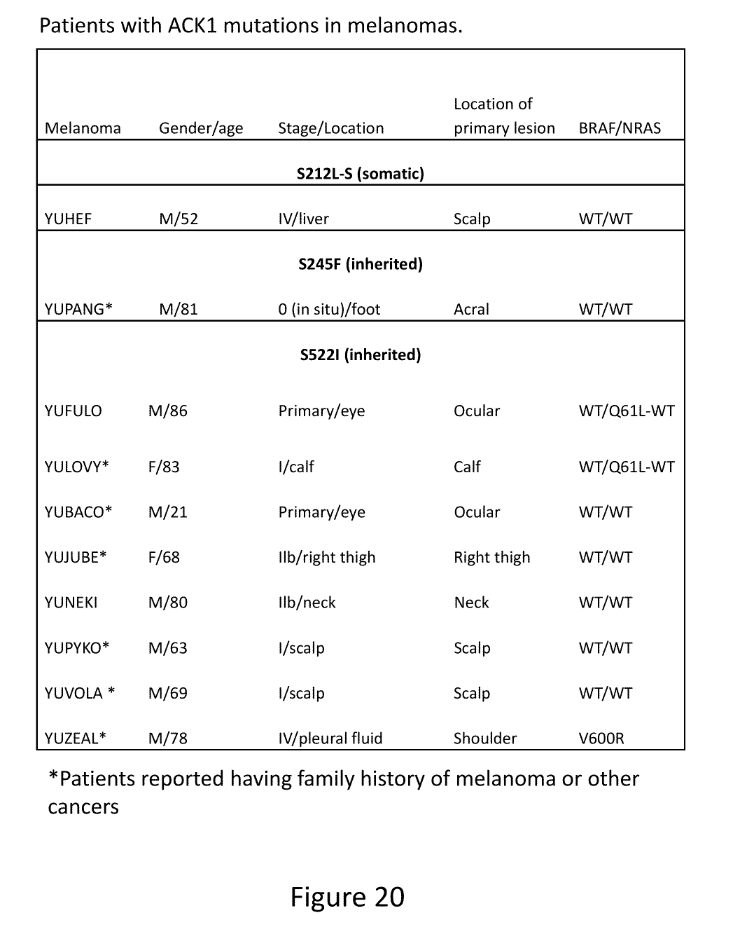

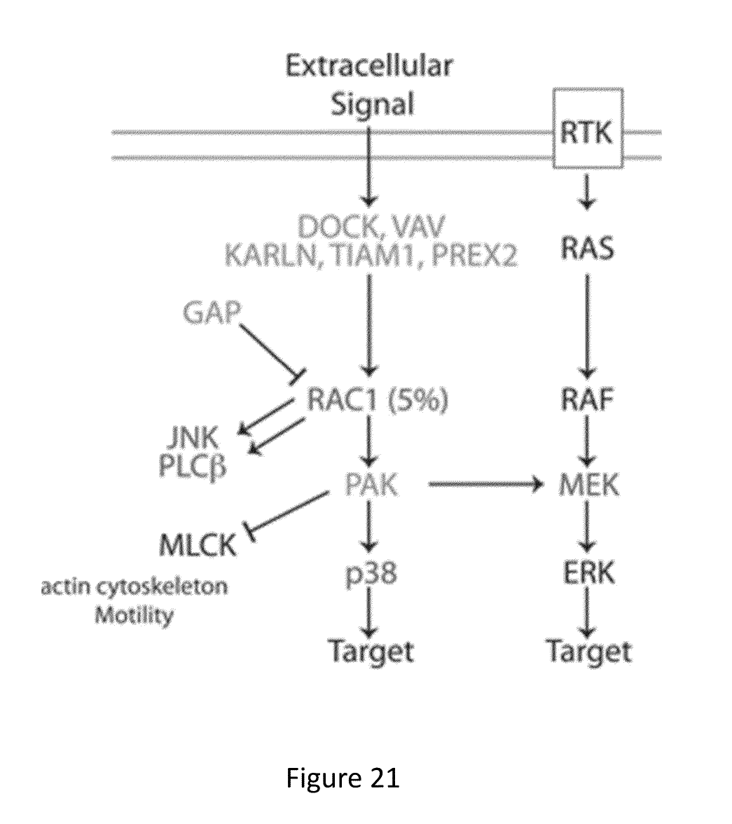

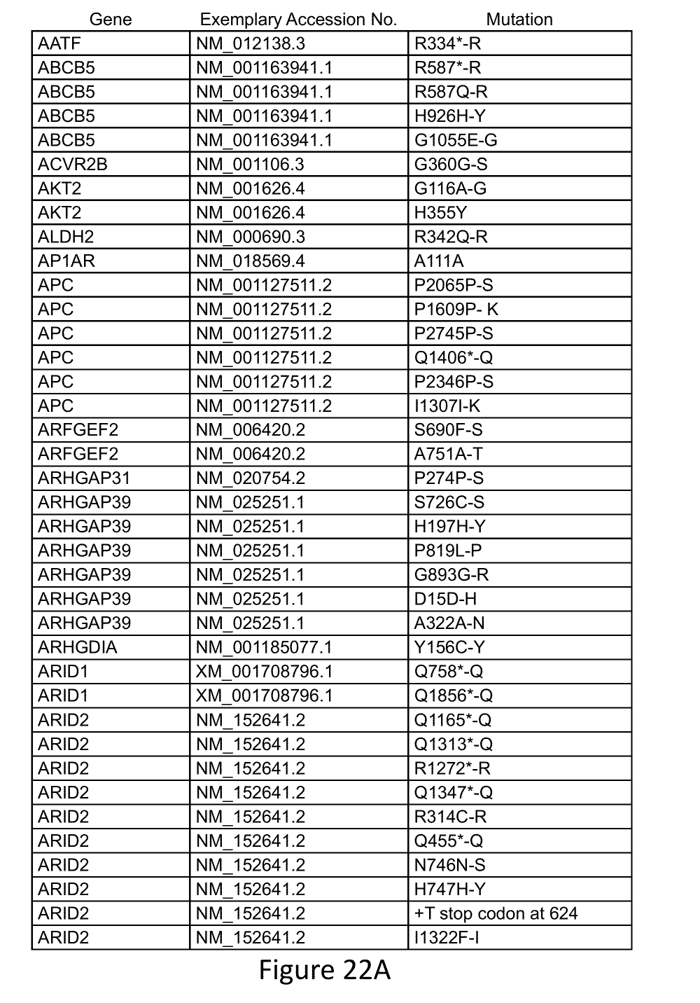

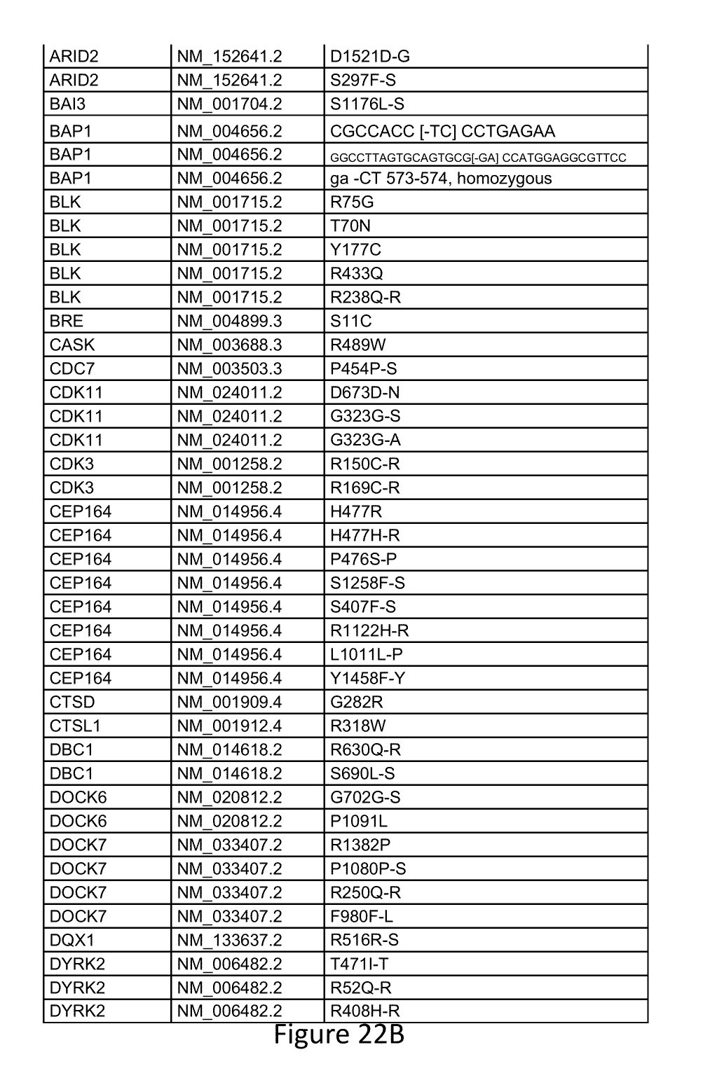

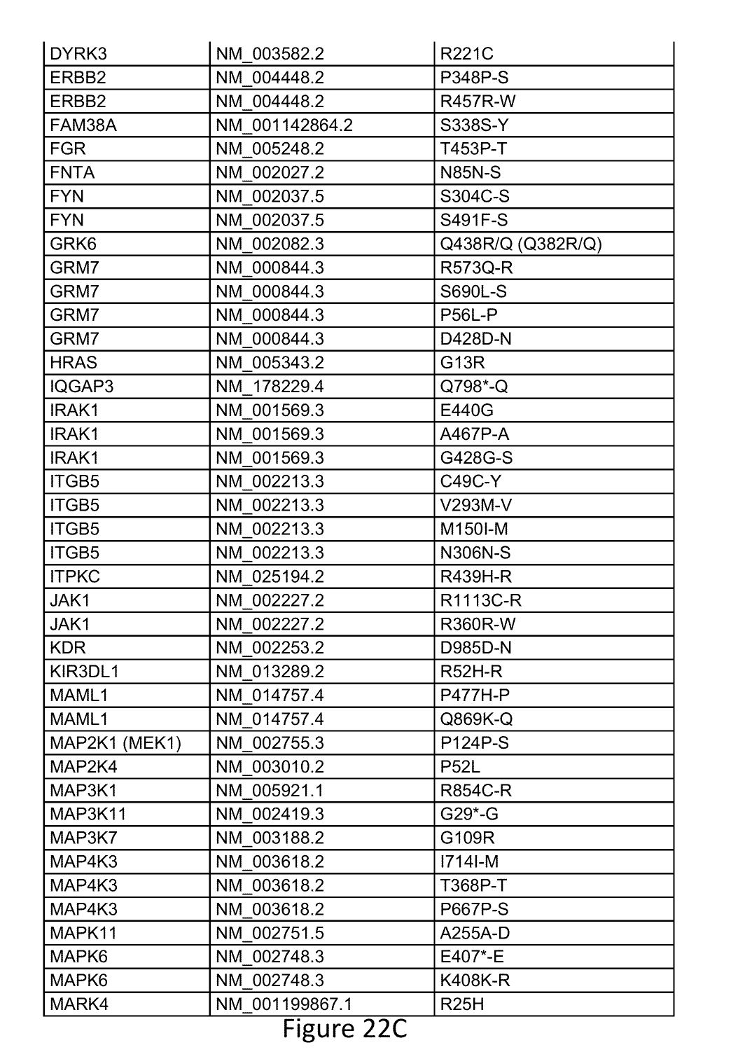

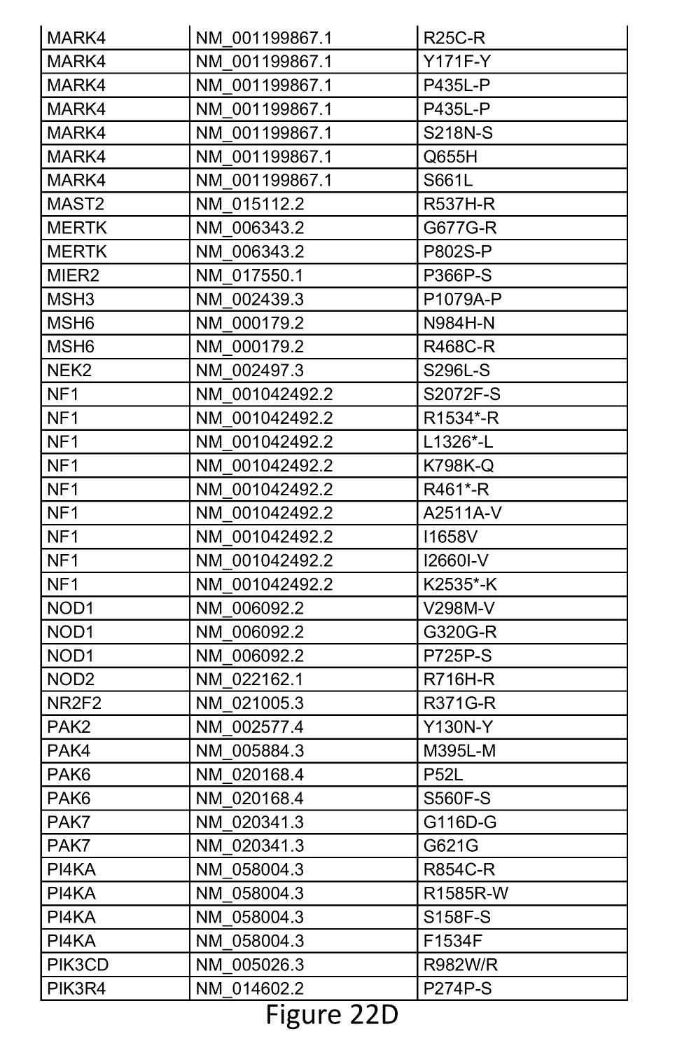

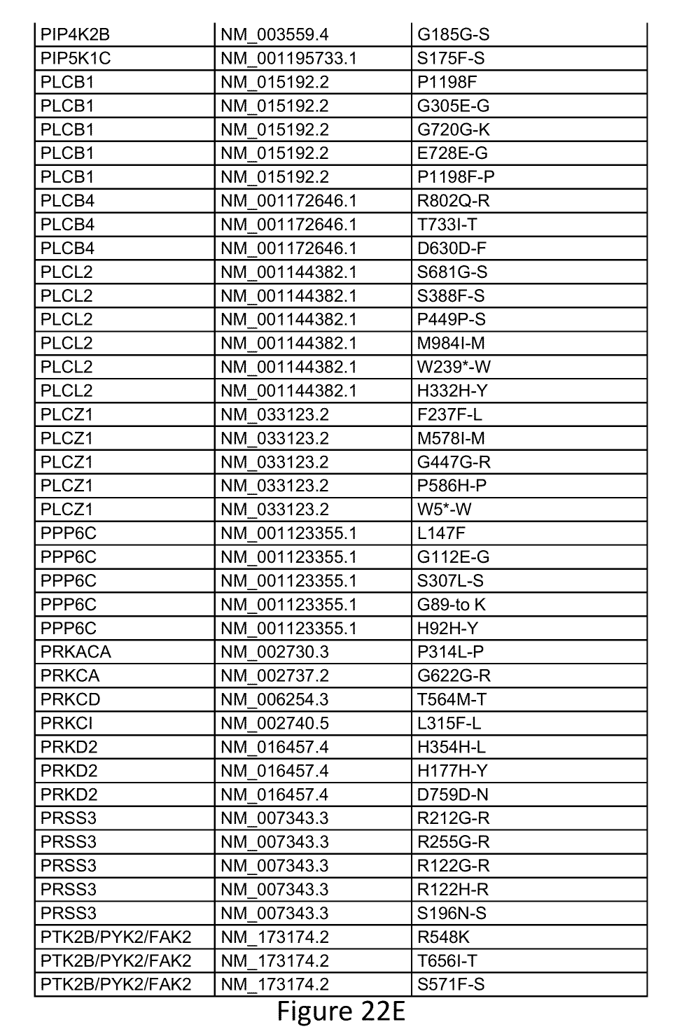

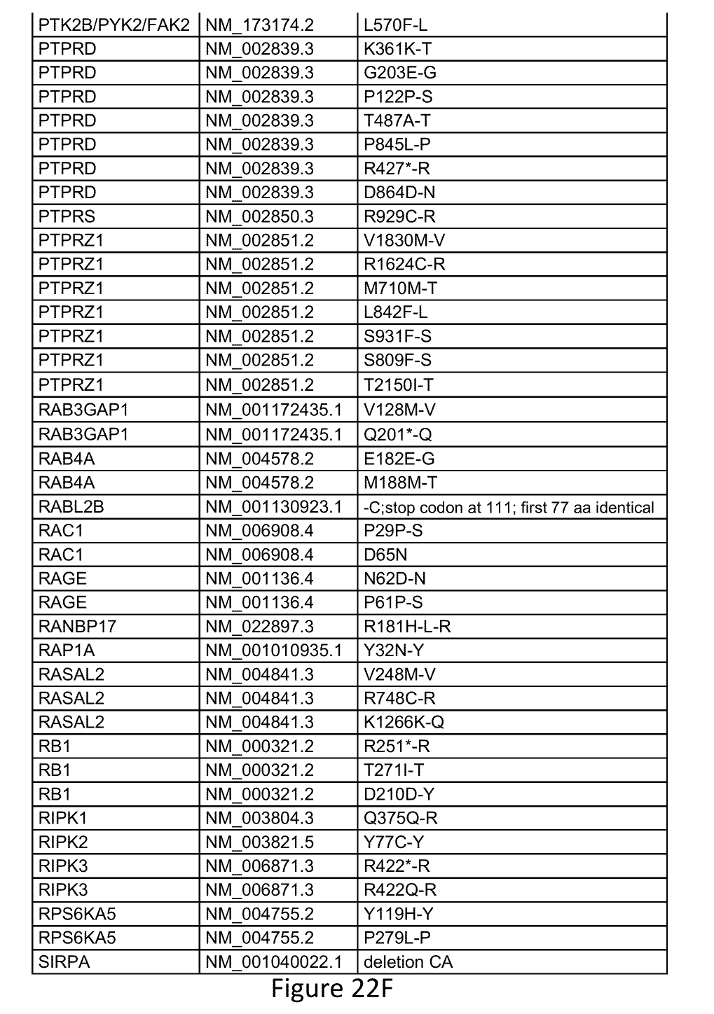

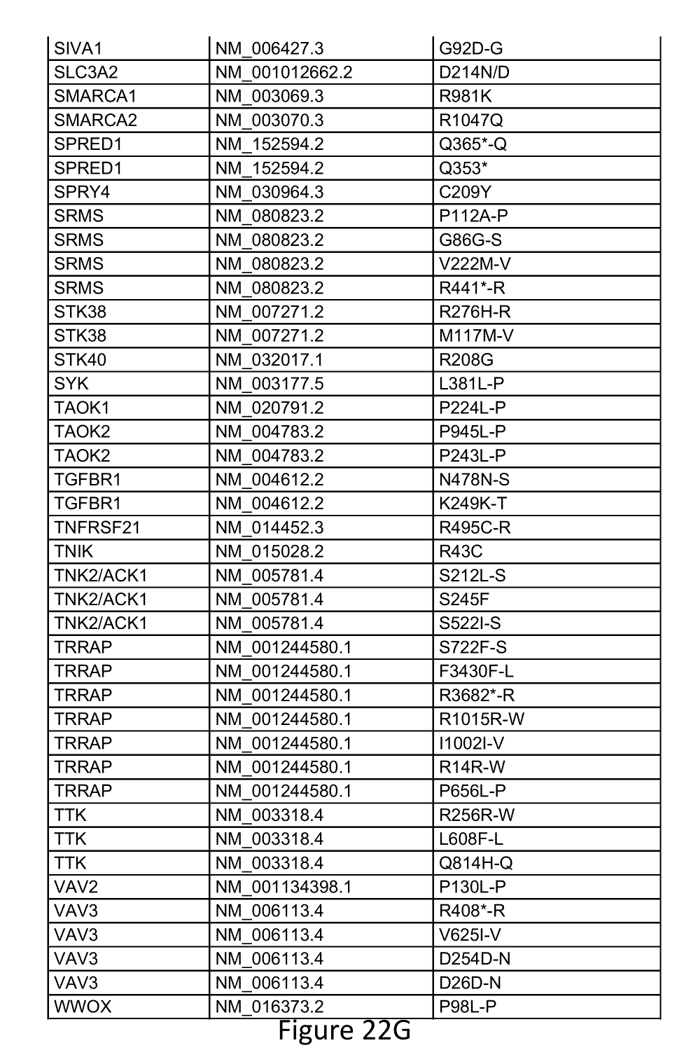

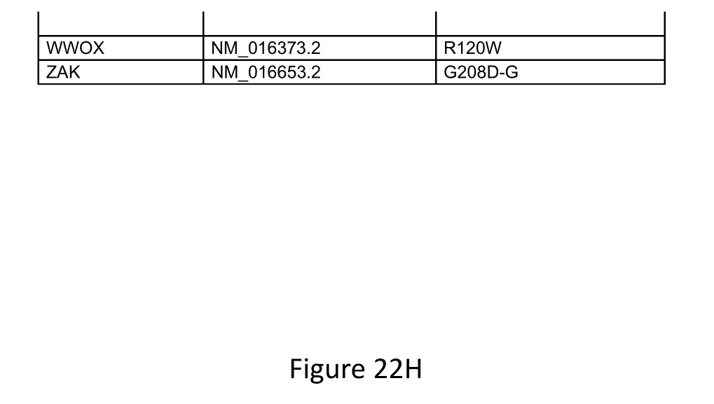

Several of the novel protein kinase SNVs that have been identified so far are inherited. For example ACK1 (official gene symbol TNK2)-S522I (NM_{—}005781.4) is the most prevalent inherited SNV in the melanoma samples described herein, particularly in patients with a family history of cancer (FIG. 20). The three ACK1 mutation/SNVs in the presently studied melanoma patients are novel (FIG. 10 arrows and FIG. 20). ACK1 S212L is a de novo somatic mutation, and based on crystal structure analysis the substitution of S212 to the larger, and hydrophobic leucine residue on the lip of the ATP-binding cleft may alter the kinetics of ATP binding to ACK1, potentially altering kinase activity. The S245F and S522I are inherited, present also in the matching germ line DNA of melanoma patients. Particular focus should be applied on candidates that lie within regions of high lod scores of previous linkage studies, or in proximity of high scoring SNPs in gene association studies (Gerstenblith et al., 2010, Pigment Cell Melanoma Res, 23:587-606).

The frequency of S522I ACK1 variant in additional melanoma specimens was determined and so far 9 SNV in 154 (5%) melanoma tumors and freshly isolated melanoma cells were identified. Interestingly, six out of the eight patients have a family history of melanoma or other cancers. Other somatic mutations in ACK1 were reported in ovarian, lung and brain cancer cells (Chua et al., 2010, Mol Oncol, 2010:323-34; Galsteo et al., 2006, Proc Natl Acad Sci USA, 103:9796-801). One particular mutation, S895N in the ubiquitin association domain (UBA, FIG. 10) enhances oncogenic signaling by preventing degradation of EGFR in renal cancer derived cells (Chua et al., 2010, Mol Oncol, 2010:323-34). ACK1 is a ubiquitously expressed non-receptor tyrosine kinase. As previously shown, it integrates signals from integrins and several growth factor activated receptors, such as EGF and PDGF to promote cell proliferation and cell survival (Galsteo et al., 2006, Proc Natl Acad Sci USA, 103:9796-801). ACK1 controls the activity of the anti-apoptotic protein kinase AKT/PKB in some cancer cells (Galsteo et al., 2006, Proc Natl Acad Sci USA, 103:9796-801; Mahajan et al., 2010, PLoS One, 5:e9646; Mahajan et al., 2010, J Cell Physiol, 224:327-33). However, the role of ACK1 in melanoma is not yet clear. In cell culture studies, PLX4032 treatment, a highly effective in vitro inhibitor of purified ACK1 (Bollag et al., 2010, Nature, 467:596-99), did not inhibit the proliferation of YUHEF melanoma cells harboring ACK1-S212L, and YULOVY melanoma cells possessing the S522I variant (Halaban et al., 2010, Pigment Cell Melanoma Res, 23:190-200). However, other tumor functions, such as cell migration and metastasis might be affected by ACK1.

In addition, ACK1 may have a role in melanoma initiation via other targets, such as the tumor suppressor WWOX (WW domain-containing oxidoreductase). ACK1 induces WWOX Tyr287 phosphorylation, which promotes its degradation via the ubiquitin/proteasome pathway (Mahajan et al., 2005, Cancer Res, 65:10514-23). WWOX is an oxidoreductase, an activity that can be critical in malignant transformation, especially in melanocytes. Melanocytes are rich in reactive oxygen species (ROS) due to tyrosinase activity, a source of hydrogen peroxide (Oetting et al., 1999, Hum Mutat, 13:99-115; Sturm, 2009, Hum Mol Genet, 18: R9-R17). High levels of ROS can lead to stochastic DNA damage and rare mutations in sunburned skin, in which high levels of reactive oxygen species are produced due to UV light and an increase in tyrosinase activity. Therefore, although not wishing to be bound by any particular theory, WWOX might be one of the protective enzymes within the cells whose downregulation (or misregulation via ACK1 variants) may enhance sun-induced DNA damage, mutations, and malignant transformation.

The ACK S522I variant is located in the non-catalytic region of ACK1 in a highly conserved region rich in proline residues (FIG. 10). It has been shown that the SH3 domain of ACK1 binds intramolecularly to the C-terminal portion of the molecule (Galisteo et al., 2006, Proc Natl Acad Sci USA, 103:9796-801). Mutations that compromise the binding activity of ACK1 SH3 domain lead to enhanced tyrosine kinase activity and autophosphorylation of ACK1 by relieving autoinhibition. This suggests that intramolecular association of the SH3 domain may have an autoinhibitory function that is relieved upon oncogenic mutations in the proline rich region of the kinase. Therefore, although not wishing to be bound by any particular theory, the S522I mutation may relieve autoinhibition that may lead to autophosphorylation of the adjacent Tyr residue and ACK1 activation.

To explore whether the frequency of the S522I variant in melanoma exceeds that of the general population and may therefore be a marker of melanoma risk, the genotype of this locus (AGC/ATC) and an adjacent known SNP rs733119 were explored in 154 melanomas compared to healthy individuals from 57 groups from around the world. This examination showed that the ATC allele (S522I) was not found in any individual in these groups, compared to 5% of the melanoma patient population. In addition, this mutation was not present in 350 other individuals under study, suggesting that this mutation may represent a unique “melanoma” gene. For context, 5-10% of all cutaneous melanomas occur in families with hereditary melanoma predisposition, and germline mutations in the major melanoma susceptibility gene, CDKN2A, are present in approximately 20-40% of kindred with familial melanoma. This means that the frequency of CDKN2A mutation in the general population is ˜2% (Hansson, 2010, Adv Exp Med Biol, 685:134-45). The S522I SNV is more frequent in patients with family history of melanomas (80%). In contrast, the frequency of patients with family history of melanoma or other cancer in the patients studied herein is 20%. The single S245F SNV was detected so far in one patient (YUPANG) whose father died from cancer (FIG. 20). Although not wishing to be bound by any particular theory, these findings are consistent with the explanation that these two variants are cancer predisposition genes.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. An isolated RAC1 polypeptide having an amino acid sequence comprising at least one mutation at amino acid position 29.

2. The isolated RAC1 polypeptide of claim 1, wherein the at least one mutation at position 29 replaces P with S.

3. An isolated RAC1 polypeptide having an amino acid sequence comprising at least one mutation at amino acid position 65.

4. The isolated RAC1 polypeptide of claim 3, wherein the at least one mutation at position 65 replaces D with N.

5. An isolated RAC1 nucleic acid having a nucleotide sequence comprising at least one mutation, wherein the nucleic acid having at least one mutation encodes a polypeptide having an amino acid sequence comprising at least one mutation at amino acid position 29.

6. The isolated RAC1 nucleic acid of claim 5, wherein the at least one mutation at position 29 replaces P with S.

7. An isolated RAC1 nucleic acid having a nucleotide sequence comprising at least one mutation, wherein the nucleic acid having at least one mutation encodes a polypeptide having an amino acid sequence comprising at least one mutation at amino acid position 65.

8. The isolated RAC1 nucleic acid of claim 7, wherein the at least one mutation at position 65 replaces D with N.

9. A method of identifying a mutant RAC1 sequence in a biological sample obtained from a subject, the method comprising:
a. obtaining a biological sample from the subject,
b. determining the sequence of the subject's RAC1 sequence,
c. comparing the subject's RAC1 sequence to a wild-type RAC1 sequence, and
d. identifying at least one mutation in the subject's RAC1 sequence as compared with the wild-type RAC1 sequence.

10. The method of claim 9, wherein the RAC1 sequence is a nucleic acid sequence, and wherein the nucleic acid sequence encodes a polypeptide having at least one mutation.

11. The method of claim 10, wherein the at least one mutation is at least one selected from the group consisting of: P29X, P29S, D65X and D65N.

12. The method of claim 9, wherein the RAC1 sequence is an amino acid sequence, and wherein the amino acid sequence comprises at least one mutation.

13. The method of claim 12, wherein the at least one mutation is at least one selected from the group consisting of: P29X, P29S, D65X and D65N.

14. The method of claim 9, wherein the subject is human.

15. The method of claim 9, wherein said step of determining the sequence of the subject's RAC1 sequence employs PCR.

16. A method of diagnosing melanoma in a subject in need thereof, the method comprising:
a. obtaining a biological sample from the subject,
b. determining the sequence of the subject's RAC1 sequence,
c. comparing the subject's RAC1 sequence to a wild-type RAC1 sequence,
d. identifying at least one mutation in the subject's RAC1 sequence as compared with the wild-type RAC1 sequence.

17. The method of claim 16, wherein the RAC1 sequence is a nucleic acid sequence, and wherein the nucleic acid sequence encodes a polypeptide having at least one mutation.

18. The method of claim 17, wherein the at least one mutation is at least one selected from the group consisting of: P29X, P29S, D65X and D65N.

19. The method of claim 16, wherein the RAC1 sequence is an amino acid sequence, and wherein the amino acid sequence comprises at least one mutation.

20. The method of claim 19, wherein the at least one mutation is at least one selected from the group consisting of: P29X, P29S, D65X and D65N.

21. The method of claim 16, wherein the subject is human.

22. The method of claim 16, wherein said step of determining the sequence of the subject's RAC1 sequence employs PCR.

23. A method of prognosing the outcome of melanoma in a subject in need thereof, the method comprising:
a. obtaining a biological sample from the subject, and
b. determining the sequence of the subject's RAC1 sequence, and
c. comparing the subject's RAC1 sequence to a wild-type RAC1 sequence, and
d. identifying at least one mutation in the subject's RAC1 sequence as compared with the wild-type RAC1 sequence.

24. The method of claim 23, wherein the RAC1 sequence is a nucleic acid sequence, and wherein the nucleic acid sequence encodes a polypeptide having at least one mutation.

25. The method of claim 24, wherein the at least one mutation is at least one selected from the group consisting of: P29X, P29S, D65X and D65N.

26. The method of claim 23, wherein the RAC1 sequence is an amino acid sequence, and wherein the amino acid sequence comprises at least one mutation.

27. The method of claim 26, wherein the at least one mutation is at least one selected from the group consisting of: P29X, P29S, D65X and D65N.

28. The method of claim 23, wherein the subject is human.

29. The method of claim 23, wherein said step of determining the sequence of the subject's RAC1 sequence employs PCR.

30. A method of determining the appropriate melanoma treatment regimen to administer to a subject in need thereof, the method comprising:
a. obtaining a biological sample from the subject,
b. determining the sequence of the subject's RAC1 sequence,
c. comparing the subject's RAC1 sequence to a wild-type RAC1 sequence,
d. identifying at least one mutation in the subject's RAC1 sequence as compared with the wild-type RAC1 sequence, and
e. selecting the appropriate melanoma treatment regimen to administer to the subject based upon the presence or absence of the at least one mutation.

31. The method of claim 30, wherein the RAC1 sequence is a nucleic acid sequence, and wherein the nucleic acid sequence encodes a polypeptide having at least one mutation.

32. The method of claim 31, wherein the at least one mutation is at least one selected from the group consisting of: P29X, P29S, D65X and D65N.

33. The method of claim 30, wherein the RAC1 sequence is an amino acid sequence, and wherein the amino acid sequence comprises at least one mutation.

34. The method of claim 33, wherein the at least one mutation is at least one selected from the group consisting of: P29X, P29S, D65X and D65N.

35. The method of claim 30, wherein the subject is human.

36. The method of claim 30, wherein said step of determining the sequence of the subject's RAC1 sequence employs PCR.

37. A method of treating melanoma in a subject in need thereof, the method comprising: administering to the subject, a therapeutically effective amount of an inhibitor of a gene associated with melanoma, wherein the subject has been diagnosed as having melanoma, and wherein after the inhibitor is administered to the subject, the melanoma is treated.

38. The method of claim 37, wherein the inhibitor is at least one selected from the group consisting of: a chemical compound, a protein, a peptide, a peptidomemetic, an antibody, a ribozyme, a small molecule chemical compound, and an antisense nucleic acid molecule.

39. The method of claim 37, wherein the gene is RAC1.

40. The method of claim 37, wherein the gene is a modulator of RAC1.

41. The method of claim 37, wherein the gene is an effector of RAC1.

42. The method of claim 37, wherein the gene is at least one selected from the group consisting of RAC1 P29X, RAC1 P29S, RAC1 D65X, and RAC1 D65N.

43. The method of claim 37, wherein the gene is a modulator of at least one selected from the group consisting of RAC1 P29X, RAC1 P29S, RAC1 D65X, and RAC1 D65N.

44. The method of claim 37, wherein the gene is an effector of at least one selected from the group consisting of RAC1 P29X, RAC1 P29S, RAC1 D65X, and RAC1 D65N.

45. The method of claim 37, wherein the subject is human.

46. A method of preventing melanoma in a subject in need thereof, the method comprising: administering to the subject, a therapeutically effective amount of an inhibitor of a gene associated with melanoma, wherein the subject has been identified as being at risk of developing melanoma, and wherein after the inhibitor is administered to the subject, the melanoma is prevented.

47. The method of claim 46, wherein the inhibitor is at least one selected from the group consisting of: a chemical compound, a protein, a peptide, a peptidomemetic, an antibody, a ribozyme, a small molecule chemical compound, and an antisense nucleic acid molecule.

48. The method of claim 46, wherein the gene is RAC1.

49. The method of claim 46, wherein the gene is a modulator of RAC1.

50. The method of claim 46, wherein the gene is an effector of RAC1.

51. The method of claim 46, wherein the gene is at least one selected from the group consisting of RAC1 P29X, RAC1 P29S, RAC1 D65X, and RAC1 D65N.

52. The method of claim 46, wherein the gene is a modulator of at least one selected from the group consisting of RAC1 P29X, RAC1 P29S, RAC1 D65X, and RAC1 D65N.

53. The method of claim 46, wherein the gene is an effector of at least one selected from the group consisting of RAC1 P29X, RAC1 P29S, RAC1 D65X, and RAC1 D65N.

54. The method of claim 46, wherein the subject is human.


SEQUENCE LISTING (16 sequences)

SEQ ID NO: 1 - DNA, 24 bp, Homo sapiens
acctaaacagaatgtgatggctcc

SEQ ID NO: 2 - DNA, 22 bp, Homo sapiens
ggtcaaagaaatgtgaaacccg

SEQ ID NO: 3 - DNA, 25 bp, Homo sapiens
aagttttgcccgtgccgccttcctc

SEQ ID NO: 4 - DNA, 26 bp, Homo sapiens
ccaatcccaccctgtttgctatttac

SEQ ID NO: 5 - DNA, 25 bp, Homo sapiens
tggtgataaagggttatagaaaaca

SEQ ID NO: 6 - DNA, 20 bp, Homo sapiens
cagcaaaacaaatggtcaaa

SEQ ID NO: 7 - protein, 211 aa, Homo sapiens
MQAIKCVVVGDGAVGKTCLLISYTTNAFPGEYIPTVFDNYSANVMVDGKPVNLGLWDTAGQEDYDRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 8 - protein, 211 aa, Homo sapiens
  misc_feature (29)..(29): Xaa can be any naturally occurring amino acid
MQAIKCVVVGDGAVGKTCLLISYTTNAFXGEYIPTVFDNYSANVMVDGKPVNLGLWDTAGQEDYDRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 9 - protein, 211 aa, Homo sapiens
MQAIKCVVVGDGAVGKTCLLISYTTNAFSGEYIPTVFDNYSANVMVDGKPVNLGLWDTAGQEDYDRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 10 - protein, 211 aa, Homo sapiens
  misc_feature (65)..(65): Xaa can be any naturally occurring amino acid
MQAIKCVVVGDGAVGKTCLLISYTTNAFPGEYIPTVFDNYSANVMVDGKPVNLGLWDTAGQEDYXRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 11 - protein, 211 aa, Homo sapiens
MQAIKCVVVGDGAVGKTCLLISYTTNAFPGEYIPTVFDNYSANVMVDGKPVNLGLWDTAGQEDYNRLRPLSYPQTVGETYGKDITSRGKDKPIADVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 12 - DNA, 42 bp, Homo sapiens
gttacacaaccaatgcattttctggagaatatatccctactg

SEQ ID NO: 13 - DNA, 42 bp, Homo sapiens
cagtagggatatattctccagaaaatgcattggttgtgtaac

SEQ ID NO: 14 - DNA, 22 bp, Homo sapiens
tttgcggataggatagggggcg

SEQ ID NO: 15 - DNA, 21 bp, Homo sapiens
tcaagtgtgtggtggtgggag

SEQ ID NO: 16 - DNA, 22 bp, Homo sapiens
tttgcggataggatagggggcg